(19) United States
(12) Patent Application Publication
(10) Pub. No.: US 2024/0376446 A1
(43) Pub. Date: Nov. 14, 2024

(54) HYPERACTIVE TRANSPOSASES

(71) Applicant: Celltheon Corporation, Union City, CA (US)

(72) Inventor: Nikhil Goel, Pleasanton, CA (US)

(73) Assignee: Celltheon Corporation, Union City, CA (US)

(21) Appl. No.: 18/658,427

(22) Filed: May 8, 2024

Related U.S. Application Data
(60) Provisional application No. 63/464,682, filed on May 8, 2023.

(51) Int. Cl. C12N 9/12

(52) U.S. Cl. CPC C12N 9/1241

(57) ABSTRACT

The present invention refers to hyperactive variants of a transposase. The invention further refers to corresponding nucleic acids producing these variants, to a gene transfer system for stably introducing nucleic acid(s) into the DNA of a cell by using these hyperactive variants of a transposase and to transposons used in the inventive gene transfer system, comprising a nucleic acid sequence with flanking repeats (IRs and/or RSDs). Furthermore, applications of these transposase variants, the transposon, or the gene transfer system are also disclosed such as gene therapy, insertional mutagenesis, gene discovery (including genome mapping), mobilization of genes, library screening, or functional analysis of genomes in vivo and in vitro. Finally, pharmaceutical compositions and kits are also encompassed.

### CROSS-REFERENCE TO RELATED APPLICATIONS

This application claims priority to U.S. Provisional Patent Application No. 63/464,682, filed on May 8, 2023, the contents of which is incorporated by reference herein in its entirety.

### INCORPORATION BY REFERENCE

In accordance with 37 CFR 1.831 (a), 37 CFR 1.835 (a) (2) and/or 1.835 (b) (2), Applicant states that the Sequence Listing filed with the application is entitled 061812_00008_SEQ_Listing.xml, was created on 2023 May 7, and is 138 KB in size, and is hereby incorporated by reference in its entirety.

### 1. FIELD OF THE INVENTION

The present invention refers to hyperactive variants of a transposase. The invention further refers to corresponding nucleic acids producing these variants, to a gene transfer system for stably introducing nucleic acid(s) into the DNA of a cell by using these hyperactive variants of a transposase and to transposons used in the inventive gene transfer system, comprising a nucleic acid sequence with flanking repeats (IRs and/or RSDs).

### 2. BACKGROUND OF THE INVENTION

The present invention refers to hyperactive variants of a transposase. The invention further refers to corresponding nucleic acids producing these variants, to a gene transfer system for stably introducing nucleic acid(s) into the DNA of a cell by using these hyperactive variants of a transposase and to transposons used in the inventive gene transfer system, comprising a nucleic acid sequence with flanking repeats (IRs and/or RSDs). Furthermore, applications of these transposase variants, the transposon, or the gene transfer system are also disclosed such as gene therapy, insertional mutagenesis, gene discovery (including genome mapping), mobilization of genes, library screening, or functional analysis of genomes in vivo and in vitro. Finally, pharmaceutical compositions and kits are also encompassed.

In the era of widespread therapeutic recombinant protein manufacturing and the nascent cell and gene therapy field, there is a sore need for developing efficient means of integrating a gene of interest into a cell line for the purpose of producing high expression levels of recombinant therapeutic product in-vitro, ex-vivo, and in-vivo. Such methods, apart from others, particularly comprise methods for introducing DNA into a cell.

Typical methods for introducing DNA into a cell include DNA condensing reagents such as calcium phosphate, polyethylene glycol, and the like, lipid-containing reagents, such as liposomes, multi-lamellar vesicles, and the like, as well as virus-mediated strategies. However, all of these methods have their limitations. For example, there are size constraints associated with DNA condensing reagents and virus-mediated strategies. Virus production and application to therapeutic product development also poses significant technical regulatory hurdles due to residual replication competent virus contamination and other product related contaminants, low productivity, and high complexity in scaleup of viral vector production. Further, the amount of nucleic acid that can be transfected into a cell is limited in virus strategies. Not all methods facilitate insertion of the delivered nucleic acid into the cellular genome and while DNA condensing methods and lipid-containing reagents are relatively easy to prepare, the insertion of the nucleic acid into the genome is not guaranteed and identification of “random gene integration” events can be labor intensive. Moreover, virus-mediated strategies can be cell-type or tissue-type specific and the use of virus-mediated strategies can create immunologic problems when used in vivo.

One suitable tool used in combination with the previously described gene transfer tools or independent from them to overcome these problems are transposons. Transposons or transposable elements include a (short) nucleic acid sequence with terminal repeat sequences upstream and downstream thereof. Active transposons encode enzymes that facilitate the excision and insertion of the nucleic acid into target DNA sequences.

### 3. SUMMARY OF THE INVENTION

DNA transposons have been developed as gene transfer vectors in invertebrate model organisms and more recently, in vertebrates too. They also rose to be strong rivals of the retroviral systems in human gene therapy. As said before the most useful transposable elements (TEs) for genetic analyses and for therapeutic approaches are the Class II TEs moving in the host genome via a “cut-and-paste” mechanism, due to their easy laboratory handling and controllable nature. PiggyBac (hereinafter abbreviated as “PB”) belongs to the family of the “cut-and-paste” transposons. These mobile DNA elements are simply organized, encoding a transposase protein in their genome flanked by the inverted terminal repeats (ITR). The ITRs carry the transposase binding sites necessary for transposition. Their activities can easily be controlled by separating the transposase source from the transposable DNA harboring the ITRs, thereby creating a non-autonomous TE. In such a two-component system, the transposon can only move by transsupplementing the transposase protein. Practically any sequence of interest can be positioned between the ITR elements according to experimental needs. The transposition will result in excision of the element from the vector DNA and subsequent single copy integration into a new sequence environment. This however may occur multiple times in a given cell at a given time if the copy number of the transposon is greater than 1.

In general the transposon mediated chromosomal entry seems to be advantageous over viral approaches because on one hand transposons if compared to viral systems do not favour so much the active genes and 5′ regulatory regions and thus are not so prone to mutagenesis, and on the other hand due to there special mechanism of chromosomal entry into of the gene of interest are more physiologically controlled.

Even though functional and valuable as commonly known and described as of today the transposase activity is likely to be one of the factors that still causes the transposon systems to reach its limits. Thus, a remarkable improvement of transpositional activity could breach current experimental barriers in both directions.

Thus, there still remains a need for improving the already valuable transposase system as a method for introducing DNA into a cell. Accordingly, it is desired to enhance efficient insertion of transposons of varying size into the nucleic acid of a cell or the insertion of DNA into the genome of a cell thus allowing more efficient transcription/translation than currently available in the state of the art.

The object underlying the present invention is solved by a polypeptide selected from a transposase comprising an additional amino acid sequence domain not natively present in the sequence of the transposase, by including at least of one insertion of a DNA binding domain selective to GC-rich DNA sequences.

### 4. DESCRIPTION OF FIGURES

FIG. 1 Human GC-Rich DNA Binding Zinc Fingers alignment

FIG. 2 Human GC-Rich DNA Binding Zinc Finger maximum likelihood phylogeny by PHYML

FIG. 3 Pairwise sequence identity between different Human GC-Rich DNA Binding Zinc Finger domains

FIG. 4 MFI and % integration efficiency of a GFP vector containing Teratorn TIRs and GFP with plasmid expressing each of the Teratorn variants described in the graph and examples.

FIG. 5 Relative expression of an antibody from a stable pool produced by co-transfecting an antibody expressing vector with Teratorn transposase variants.

FIG. 6 Relative expression levels of an antibody from a stable pool produced by co-transfecting an antibody expressing vector with PiggyBac transposase variants or no transposase.

FIG. 7 shows integration efficiency as compared to those without a tetramerization domain.

FIG. 8 shows the additional increase in expression of transposase by PB-CTVar14+Gpbp1+Tetramerizer_SEP3 and PB-CTVar14+Sp1_ZFN+Tetramerizer_SEP3 as compared to the PB_CTVar14 pool.

### 5. DETAILED DESCRIPTION OF THE INVENTION

### 4.1 Definitions

Use of the singular forms “a,” “an,” and “the” include plural references unless the context clearly dictates otherwise. Thus, for example, reference to “a polynucleotide” includes a plurality of polynucleotides, reference to “a substrate” includes a plurality of such substrates, reference to “a variant” includes a plurality of variants, and the like.

Terms such as “connected,” “attached,” “linked,” and “conjugated” are used interchangeably herein and encompass direct as well as indirect connection, attachment, linkage or conjugation unless the context clearly dictates otherwise. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the invention. Where a value being discussed has inherent limits, for example where a component can be present at a concentration of from 0 to 100%, or where the pH of an aqueous solution can range from 1 to 14, those inherent limits are specifically disclosed. Where a value is explicitly recited, it is to be understood that values which are about the same quantity or amount as the recited value are also within the scope of the invention. Where a combination is disclosed, each sub combination of the elements of that combination is also specifically disclosed and is within the scope of the invention. Conversely, where different elements or groups of elements are individually disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

Unless defined otherwise herein, all technical and scientific terms used herein have the same meaning as commonly understood by one of ordinary skill in the art to which this invention belongs. Singleton, et al., Dictionary of Microbiology and Molecular Biology, 2nd Ed., John Wiley and Sons, New York (1994), and Hale & Marham, The Harper Collins Dictionary of Biology, Harper Perennial, NY, 1991, provide one of skill with a general dictionary of many of the terms used in this invention. Although any methods and materials similar or equivalent to those described herein can be used in the practice or testing of the present invention, the preferred methods and materials are described. Unless otherwise indicated, nucleic acids are written left to right in 5′ to 3′ orientation; amino acid sequences are written left to right in amino to carboxy orientation, respectively. The terms defined immediately below are more fully defined by reference to the specification as a whole.

The “configuration” of a polynucleotide means the functional sequence elements within the polynucleotide, and the order and direction of those elements.

The terms “corresponding transposon” and “corresponding transposase” are used to indicate an activity relationship between a transposase and a transposon. A transposase transposes its corresponding transposon. Many transposases may correspond with a single transposon. A transposon is transposed by its corresponding transposase. Many transposons may correspond with a single transposase.

The term “counter-selectable marker” means a polynucleotide sequence that confers a selective disadvantage on a host cell. Examples of counter-selectable markers include sacB, rpsL, tetAR, pheS, thyA, gata-1, ccdB, kid and barnase (Bernard, 1995, Journal/Gene, 162:159-160; Bernard et al., 1994. Journal/Gene, 148:71-74; Gabant et al., 1997, Journal/Biotechniques, 23:938-941; Gababt et al., 1998, Journal/Gene, 207:87-92; Gababt et al., 2000, Journal/Biotechniques, 28:784-788; Galvao and de Lorenzo, 2005, Journal/Appl Environ Microbiol, 71:883-892; Hartzog et al., 2005, Journal/Yeat, 22:789-798; Knipfer et al., 1997, Journal/Plasmid, 37:129-140; Reyrat et al., 1998, Journal/Infect Immun, 66:4011-4017; Soderholm et al., 2001, Journal/Biotechniques, 31:306-310, 312; Tamura et al., 2005, Journal/Appl Environ Microbiol, 71:587-590; Yazynin et al., 1999, Journal/FEBS Lett, 452:351-354). Counter-selectable markers often confer their selective disadvantage in specific contexts. For example, they may confer sensitivity to compounds that can be added to the environment of the host cell, or they may kill a host with one genotype but not kill a host with a different genotype. Conditions which do not confer a selective disadvantage on a cell carrying a counter-selectable marker are described as “permissive”. Conditions which do confer a selective disadvantage on a cell carrying a counter-selectable marker are described as “restrictive”.

The term “coupling element” or “translational coupling element” means a DNA sequence that allows the expression of a first polypeptide to be linked to the expression of a second polypeptide. Internal ribosome entry site elements (IRES elements) and cis-acting hydrolase elements (CHYSEL elements) are examples of coupling elements.

The terms “DNA sequence”, “RNA sequence” or “polynucleotide sequence” mean a contiguous nucleic acid sequence. The sequence can be an oligonucleotide of 2 to 20 nucleotides in length to a full length genomic sequence of thousands or hundreds of thousands of base pairs.

The term “expression construct” means any polynucleotide designed to transcribe an RNA. For example, a construct that contains at least one promoter which is or may be operably linked to a downstream gene, coding region, or polynucleotide sequence (for example, a cDNA or genomic DNA fragment that encodes a polypeptide or protein, or an RNA effector molecule, for example, an antisense RNA, triplex-forming RNA, ribozyme, an artificially selected high affinity RNA ligand (aptamer), a double-stranded RNA, for example, an RNA molecule comprising a stem-loop or hairpin dsRNA, or a bi-finger or multi-finger dsRNA or a microRNA, or any RNA). An “expression vector” is a polynucleotide comprising a promoter which can be operably linked to a second polynucleotide. Transfection or transformation of the expression construct into a recipient cell allows the cell to express an RNA effector molecule, polypeptide, or protein encoded by the expression construct. An expression construct may be a genetically engineered plasmid, virus, recombinant virus, or an artificial chromosome derived from, for example, a bacteriophage, adenovirus, adeno-associated virus, retrovirus, lentivirus, poxvirus, or herpesvirus. Such expression vectors can include sequences from bacteria, viruses or phages. Such vectors include chromosomal, episomal and virus-derived vectors, for example, vectors derived from bacterial plasmids, bacteriophages, yeast episomes, yeast chromosomal elements, and viruses, vectors derived from combinations thereof, such as those derived from plasmid and bacteriophage genetic elements, cosmids and phagemids. An expression construct can be replicated in a living cell, or it can be made synthetically. For purposes of this application, the terms “expression construct”, “expression vector”, “vector”, and “plasmid” are used interchangeably to demonstrate the application of the invention in a general, illustrative sense, and are not intended to limit the invention to a particular type of expression construct.

The term “expression polypeptide” means a polypeptide encoded by a gene on an expression construct.

The term “expression system” means any in vivo or in vitro biological system that is used to produce one or more gene product encoded by a polynucleotide.

A “gene transfer system” comprises a vector or gene transfer vector, or a polynucleotide comprising the gene to be transferred which is cloned into a vector (a “gene transfer polynucleotide” or “gene transfer construct”). A gene transfer system may also comprise other features to facilitate the process of gene transfer. For example, a gene transfer system may comprise a vector and a lipid or viral packaging mix for enabling a first polynucleotide to enter a cell, or it may comprise a polynucleotide that includes a transposon and a second polynucleotide sequence encoding a corresponding transposase to enhance productive genomic integration of the transposon. The transposases and transposons of a gene transfer system may be on the same nucleic acid molecule or on different nucleic acid molecules. The transposase of a gene transfer system may be provided as a polynucleotide or as a polypeptide.

Two elements are “heterologous” to one another if not naturally associated. For example, a nucleic acid sequence encoding a protein linked to a heterologous promoter means a promoter other than that which naturally drives expression of the protein. A heterologous nucleic acid flanked by transposon ends or ITRs means a heterologous nucleic acid not naturally flanked by those transposon ends or ITRs, such as a nucleic acid encoding a polypeptide other than a transposase, including an antibody heavy or light chain. A nucleic acid is heterologous to a cell if not naturally found in the cell or if naturally found in the cell but in a different location (e.g., episomal or different genomic location) than the location described.

The term “host” means any prokaryotic or eukaryotic organism that can be a recipient of a nucleic acid. A “host,” as the term is used herein, includes prokaryotic or eukaryotic organisms that can be genetically engineered. For examples of such hosts, see Maniatis et al., Molecular Cloning. A Laboratory Manual, Cold Spring Harbor Laboratory, Cold Spring Harbor, N.Y. (1982). As used herein, the terms “host,” “host cell,” “host system” and “expression host” can be used interchangeably.

A “hyperactive” transposase is a transposase that is more active than the naturally occurring transposase from which it is derived. “Hyperactive” transposases are thus not naturally occurring sequences. For example, hyperactive transposases are transposases that upon modification become more active than their respective naturally occurring sequence variants including but not limited to SEQ ID NO: 5-13.

An “IRES” or “internal ribosome entry site” means a specialized sequence that directly promotes ribosome binding, independent of a cap structure.

An “isolated” polypeptide or polynucleotide means a polypeptide or polynucleotide that has been either removed from its natural environment, produced using recombinant techniques, or chemically or enzymatically synthesized. Polypeptides or polynucleotides of this invention may be purified, that is, essentially free from any other polypeptide or polynucleotide and associated cellular products or other impurities.

The terms “nucleoside” and “nucleotide” include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides can also include modifications on the sugar moiety, for example, where one or more of the hydroxyl groups are replaced with halogen, aliphatic groups, or is functionalized as ethers, amines, or the like. The term “nucleotidic unit” is intended to encompass nucleosides and nucleotides.

An “Open Reading Frame” or “ORF” means a portion of a polynucleotide that, when translated into amino acids, contains no stop codons. The genetic code reads DNA sequences in groups of three base pairs, which means that a double-stranded DNA molecule can read in any of six possible reading frames-three in the forward direction and three in the reverse. An ORF typically also includes an initiation codon at which translation may start.

The term “operably linked” refers to functional linkage between two sequences such that one sequence modifies the behavior of the other. For example, a first polynucleotide comprising a nucleic acid expression control sequence (such as a promoter, IRES sequence, enhancer or array of transcription factor binding sites) and a second polynucleotide are operably linked if the first polynucleotide affects transcription and/or translation of the second polynucleotide. Similarly, a first amino acid sequence comprising a secretion signal or a subcellular localization signal and a second amino acid sequence are operably linked if the first amino acid sequence causes the second amino acid sequence to be secreted or localized to a subcellular location.

The term “overhang” or “DNA overhang” means the single-stranded portion at the end of a double-stranded DNA molecule. Complementary overhangs are those which will base-pair with each other.

A “piggyBac-like transposase” means a transposase with at least 20% sequence identity as identified using the TBLASTN algorithm to the piggyBac transposase from Trichoplusia ni (SEQ ID NO: 1), and as more fully described in Sakar, A. et. al., (2003). Mol. Gen. Genomics 270:173-180. “Molecular evolutionary analysis of the widespread piggyBac transposon family and related ‘domesticated’ species”, and further characterized by a DDE-like DDD motif, with aspartate residues at positions corresponding to D268, D346, and D447 of Trichoplusia ni piggyBac transposase on maximal alignment. PiggyBac-like transposases are also characterized by their ability to excise their transposons precisely with a high frequency. A “piggyBac-like transposon” means a transposon having transposon ends which are the same or at least 80% and preferably at least 90, 95, 96, 97, 98 or 99% identical to the transposon ends of a naturally occurring transposon that encodes a piggyBac-like transposase. A piggyBac-like transposon includes an inverted terminal repeat (ITR) sequence of approximately 12-16 bases at each end, and is flanked on each side by a 4 base sequence corresponding to the integration target sequence which is duplicated on transposon integration (the Target Site Duplication or Target Sequence Duplication or TSD). PiggyBac-like transposons and transposases occur naturally in a wide range of organisms including Argyrogramma agnate (GU477713), Anopheles gambiae (XP_312615; XP_320414; XP_310729), Aphis gossypii (GU329918), Acyrthosiphon pisum (XP_001948139), Agrotis ypsilon (GU477714), Bombyx mori (BAD11135), Ciona intestinalis (XP_002123602), Chilo suppressalis (JX294476), Drosophila melanogaster (AAL39784), Daphnia pulicaria (AAM76342), Helicoverpa armigera (ABS18391), Homo sapiens (NP_689808), Heliothis virescens (ABD76335), Macdunnoughia crassisigna (EU287451), Macaca fascicularis (AB179012), Mus musculus (NP_741958), Pectinophora gossypiella (GU270322), Rattus norvegicus (XP_220453), Tribolium castaneum (XP_001814566), Trichoplusia ni (AAA87375) and Xenopus tropicalis (BAF82026).

The terms “polynucleotide,” “oligonucleotide,” “nucleic acid” and “nucleic acid molecule” and “gene” are used interchangeably to refer to a polymeric form of nucleotides of any length, and may comprise ribonucleotides, deoxyribonucleotides, analogs thereof, or mixtures thereof. This term refers only to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), including tRNA, rRNA, hRNA, siRNA, circular RNA and mRNA, whether spliced or unspliced, any other type of polynucleotide which is an N- or C-glycoside of a purine or pyrimidine base, and other polymers containing nonnucleotidic backbones, for example, polyamide (for example, peptide nucleic acids (“PNAs”)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms are used interchangeably herein. These terms refer only to the primary structure of the molecule. Thus, these terms include, for example, 3′-deoxy-2′, 5′-DNA, oligodeoxyribonucleotide N3′ P5′ phosphoramidates, 2′-O-alkyl-substituted RNA, double- and single-stranded DNA, as well as double- and single-stranded RNA, and hybrids thereof including for example hybrids between DNA and RNA or between PNAs and DNA or RNA, and also include known types of modifications, for example, labels, alkylation, “caps,” substitution of one or more of the nucleotides with an analog, internucleotide modifications such as, for example, those with uncharged linkages (for example, methyl phosphonates, phosphotriesters, phosphoramidates, carbamates, or the like) with negatively charged linkages (for example, phosphorothioates, phosphorodithioates, or the like), and with positively charged linkages (for example, aminoalkylphosphoramidates, aminoalkylphosphotriesters), those containing pendant moieties, such as, for example, proteins (including enzymes (for example, nucleases), toxins, antibodies, signal peptides, poly-L-lysine, or the like), those with intercalators (for example, acridine, psoralen, or the like), those containing chelates (of, for example, metals, radioactive metals, boron, oxidative metals, or the like), those containing alkylators, those with modified linkages (for example, alpha anomeric nucleic acids, or the like), as well as unmodified forms of the polynucleotide or oligonucleotide.

A “promoter” means a nucleic acid sequence sufficient to direct transcription of an operably linked nucleic acid molecule. Also included in this definition are those transcription control elements (for example, enhancers) that are sufficient to render promoter-dependent gene expression controllable in a cell type-specific, tissue-specific, or temporal-specific manner, or that are inducible by external signals or agents; such elements, may be within the 3′ region of a gene or within an intron. Desirably, a promoter is operably linked to a nucleic acid sequence, for example, a cDNA or a gene sequence, or an effector RNA coding sequence, in such a way as to enable expression of the nucleic acid sequence, or a promoter is provided in an expression cassette into which a selected nucleic acid sequence to be transcribed can be conveniently inserted.

The term “selectable marker” means a polynucleotide segment that allows one to select for or against a molecule or a cell that contains it, often under particular conditions. These markers can encode an activity, such as, but not limited to, production of RNA, peptide, or protein, or can provide a binding site for RNA, peptides, proteins, inorganic and organic compounds or compositions. Examples of selectable markers include but are not limited to: (1) DNA segments that encode products which provide resistance against otherwise toxic compounds (e.g., antibiotics); (2) DNA segments that encode products which are otherwise lacking in the recipient cell (e.g., tRNA genes, auxotrophic markers); (3) DNA segments that encode products which suppress the activity of a gene product; (4) DNA segments that encode products which can be readily identified (e.g., phenotypic markers such as beta-galactosidase, green fluorescent protein (GFP), and cell surface proteins); (5) DNA segments that bind products which are otherwise detrimental to cell survival and/or function; (6) DNA segments that otherwise inhibit the activity of any of the DNA segments described in Nos. 1-5 above (e.g., antisense oligonucleotides); (7) DNA segments that bind products that modify a substrate (e.g. restriction endonucleases); (8) DNA segments that can be used to isolate a desired molecule (e.g. specific protein binding sites); (9) DNA segments that encode a specific nucleotide sequence which can be otherwise non-functional (e.g., for PCR amplification of subpopulations of molecules); and/or (10) DNA segments, which when absent, directly or indirectly confer sensitivity to particular compounds.

Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences.

A “target nucleic acid” is a nucleic acid into which a transposon is to be inserted. Such a target can be part of a chromosome, episome or vector.

A “transgene” or “gene of interest” is the essential component of a nucleic acid species that encodes an RNA or protein product which effects a desired function not previously present in the target cell.

An “integration target sequence” or “target sequence” or “target site” for a transposase is a site or sequence in a target DNA molecule into which a transposon can be inserted by a transposase. For example, the piggyBac transposase from Trichoplusia ni inserts its transposon predominantly into the target sequence 5′-TTAA-3′. PiggyBac-like transposases transpose their transposons using a cut-and-paste mechanism, which results in duplication of their 4 base pair target sequence on insertion into a DNA molecule. The target sequence is thus found on each side of an integrated piggyBac-like transposon.

The term “translation” refers to the process by which a polypeptide is synthesized by a ribosome ‘reading’ the sequence of a polynucleotide.

A “transposase” is a polypeptide that catalyzes the excision of a corresponding transposon from a donor polynucleotide, for example a vector, and (providing the transposase is not integration-deficient) the subsequent integration of the transposon into a target nucleic acid. At present, two clearly defined classes of transposons are known, i.e. class I and class II.

Class I transposons, also called retrotransposons, include retroviral-like retrotransposons and non-retroviral-like retrotransposons. They work by copying themselves and pasting copies back into the genome in multiple places. Initially, retrotransposons copy themselves to RNA (transcription) but, instead of being translated, the RNA is copied into DNA by a reverse transcriptase (often coded by the transposon itself) and inserted back into the genome. Typical representatives of class I transposons include e.g. Copia (Drosophila), Ty1 (yeast), THE-1 (human), Bs1 (maize), the F-element, L1 (human) or Cin4 (maize).

Class II transposons, also called “DNA-only transposons”, generally move by a cut and paste mechanism, rather than by copy and paste, and use the transposase enzyme in this mechanism. This is not true for the helitron transposase systems which are copy and paste DNA transposons. Different types of transposases may work in different ways. Some can bind to any part of the DNA molecule, and the target site can be located at any position, while others bind to specific sequences. The transposase then cuts the target site to produce sticky ends, releases the transposon and ligates it into the target site. Typical class II representatives include the P element (Drosophila), Ac-Ds (maize), TN3 and IS1 (E. coli), Tam3 (snapdragon) etc.

Particularly, with class II transposons, the element-encoded transposase catalyzes the excision of the transposon from its original location and promotes its insertion elsewhere in the genome (Plasterk, 1996 Curr. Top. Microbiol. Immunol. 204, 125-143). Autonomous members of a transposon family can express an active transposase, the transacting factor for transposition, and thus are capable of transposing on their own. Non-autonomous elements have mutated transposase genes but may retain cis-acting DNA sequences. These cis-acting DNA sequences are also referred to as inverted terminal repeats (IR). Some inverted repeat sequences may include one or more direct repeat sequences. These sequences usually are embedded in the terminal inverted repeats (IRs) of the elements, which are required for mobilization in the presence of a complementary transposase from another element. Most transposon-like sequences found in nature are defective, apparently as a result of a process called “vertical inactivation” (Lohe et al., 1995 Mol. Biol. Evol. 12, 62-72). According to one phylogenetic model (Hartl et al., 1997 Trends Genet. 13, 197-201), the ratio of non-autonomous to autonomous elements in eukaryotic genomes increases as a result of the trans-complementary nature of transposition. This process leads to a state where the ultimate disappearance of active, transposase-producing copies in a genome is inevitable. Consequently, DNA-transposons can be viewed as transitory components of genomes which, in order to avoid extinction, must find ways to establish themselves in a new host. Indeed, horizontal gene transmission between species is thought to be one of the important processes in the evolution of transposons (Lohe et al., 1995 supra and Kidwell, 1992. Curr. Opin. Genet Dev. 2, 868-873).

Transposon systems as discussed above may occur in vertebrate and invertebrate systems. In vertebrates, the discovery of DNA-transposons, mobile elements that move via a DNA intermediate, is relatively recent (Radice, A. D., et al., 1994. Mol. Gen. Genet. 244, 606-612). Since then, inactive, highly mutated members of the Tcl/mariner as well as the hAT (hobo/AcTam) superfamilies of eukaryotic transposons have been isolated from different fish species, Xenopus and human genomes (Oosumi et al., 1995. Nature 378, 873; Ivics et al. 1995. Mol. Gen. Genet. 247, 312-322; Koga et al., 1996. Nature 383, 30; Lam et al., 1996. J. Mol. Biol. 257, 359-366 and Lam, W. L., et al. Proc. Natl. Acad Sci. USA 93, 10870-10875).

Both invertebrate and vertebrate transposons hold potential for transgenesis and insertional mutagenesis in model organisms. Particularly, the availability of alternative transposon systems in the same species opens up new possibilities for genetic analyses. For example, piggyBac transposons can be mobilized in Drosophila in the presence of stably inserted P elements (Hacker et al., (2003), Proc Natl Acad Sci USA 100, 7720-5.). Because P element- and pigyBac-based systems show different insertion site preferences (Spradling et al. (1995), Proc Natl Acad Sci USA 92, 10824-30, Hacker et al., (2003), Proc Natl Acad Sci USA 100, 7720-5), the number of fly genes that can be insertionally inactivated by transposons can greatly be increased. P element vectors have also been used to insert components of the mariner transposon into the D. melanogaster genome by stable germline transformation. In these transgenic flies, mariner transposition can be studied without accidental mobilization of P elements (Lohe and Hartl, (2002), Genetics 160, 519-26).

In vertebrates, multiple active/resurrected transposons are currently studied and used for genetic engineering: the Tol2 element in medaka (Koga et al., Nature, 1996), the Teratorn transposase also recently discovered in medaka (Takeda et al., Nat Comm, 2017), the reconstructed transposons Sleeping Beauty (SB), Frog Prince (FP) as well as the PiggyBac transposon system (Ding et al., Cell, 2005), the Hermes transposase from Musca domestica (Subramanian et al, J. Hered, 2009), the TcBuster transposase from Tribolium castaneum (Woodard et al, PLos One, 2012), a helitron transposase, Helraiser, has also been reconstructed and used for genetic engineering (Grabundzija et al., Nat Comm, 2016), and homologues thereof.

The Tol2 element is an active member of the hAT transposon family in medaka. It was discovered by a recessive mutation causing an albino phenotype of the Japanese medaka (Oryzias latipes), a small freshwater fish of East Asia. It was found that the mutation is due to a 4.7-kb long TE insertion into the fifth exon of the tyrosinase gene. The DNA sequence of the element, named Tol2, is similar to transposons of the hAT family, including hobo of Drosophila, Ac of maize and Tam3 of snapdragon.

Sleeping Beauty (SB) is a Tcl/mariner-like element from fish and exhibits high transpositional activity in a variety of vertebrate cultured cell lines, embryonic stem cells and in both somatic and germ line cells of the mouse in vivo.

Also Frog Prince (FP) is a Tcl/mariner-like element that was recently reactivated from genomic transposon copies of the Northern Leopard Frog (Rana pipiens). An open reading frame trapping method was used to identify uninterrupted transposase coding regions, and the majority rule consensus of these sequences revealed an active transposase gene.

The Teratorn transposase was found to be an active transposase identified in the Medaka genome. One of the unique features of Teratorn is its size (˜180-kb long), by far the biggest reported for a transposon.

Transposons as the above, do not interact and thus may be used as a genetic tool in the presence of others, which considerably broadens the utility of these elements. The preferences of these transposons to insert into expressed genes versus non-coding DNA, and preferences for insertion sites within genes may be substantially different. If so, different patterns of insertion of these transposon systems can be exploited in a complementary fashion. Or further, these distinct transposase systems may each be independently modified to contain domains of the other or entirely new domains which may further modify insertion patterns of the transposon.

The term “transposition” is used herein to mean the action of a transposase in excising a transposon from one polynucleotide and then integrating it, either into a different site in the same polynucleotide, or into a second polynucleotide.

The term “transposon” means a polynucleotide that can be excised from a first polynucleotide, for instance, a vector, and be integrated into a second position in the same polynucleotide, or into a second polynucleotide, for instance, the genomic or extrachromosomal DNA of a cell, by the action of a corresponding trans-acting transposase. A transposon comprises a first transposon end and a second transposon end, which are polynucleotide sequences recognized by and transposed by a transposase. A transposon usually further comprises a first polynucleotide sequence between the two transposon ends, such that the first polynucleotide sequence is transposed along with the two transposon ends by the action of the transposase. Natural transposons frequently comprise DNA encoding a transposase that acts on the transposon. Transposons of the present invention are “synthetic transposons” comprising a heterologous polynucleotide sequence which is transposable by virtue of its juxtaposition between two transposon ends.

The term “transposon end” means the cis-acting nucleotide sequences that are sufficient for recognition by and transposition by a corresponding transposase. Transposon ends of piggyBac-like transposons comprise perfect or imperfect repeats such that the respective repeats in the two transposon ends are reverse complements of each other. These are referred to as inverted terminal repeats (ITR) or terminal inverted repeats (TIR). A transposon end may or may not include additional sequence proximal to the ITR that promotes or augments transposition.

The term “GC-rich” means a region of a nucleic acid sequence containing >80% GC-content in at least a 5-bp window.

The term “GC-rich DNA Binding Domain” means a domain such as a nucleic acid (eg. DNA or RNA or functional equivalents thereof), polypeptides, or small molecules which preferentially bind to GC-rich DNA sequences.

The term “GC-rich binding protein” means a polypeptide sequence that preferentially binds to GC-rich nucleic acid sequences over AT-rich or AT/GC-neutral sequences. Nonlimiting examples of GC-rich binding proteins are provided in SEQ ID No: 14-71. Homologues with >70%, 80%, 90%, 95% sequence identity to these sequences may also be functionally equivalent sequences.

The term “vector” or “DNA vector” or “gene transfer vector” refers to a polynucleotide that is used to perform a “carrying” function for another polynucleotide. For example, vectors are often used to allow a polynucleotide to be propagated within a living cell, or to allow a polynucleotide to be packaged for delivery into a cell, or to allow a polynucleotide to be integrated into the genomic DNA of a cell. A vector may further comprise additional functional elements, for example it may comprise a transposon.

Expression of a gene from a heterologous polynucleotide in a eukaryotic host cell can be improved if the heterologous polynucleotide is integrated into the genome of the host cell. Integration of a polynucleotide into the genome of a host cell also generally makes it stably heritable, by subjecting it to the same mechanisms that ensure the replication and division of genomic DNA. Such stable heritability is desirable for achieving good and consistent expression over long growth periods. For manufacturing of biomolecules and engineered cell products, particularly for therapeutic applications, the stability of the host and consistency of expression levels is also important for regulatory purposes. Cells with gene transfer vectors, including transposon-based gene transfer vectors, integrated into their genomes are thus an important aspect of the invention.

Heterologous polynucleotides may be more efficiently integrated into a target genome if they are part of a transposon, for example so that they may be integrated by a transposase. The piggyBac transposon from the looper moth Trichoplusia ni has been shown to be transposed by its transposase in cells from many organisms (Keith et al., BMC Mol Bio, 2008). Heterologous polynucleotides incorporated into piggyBac-like transposons may be integrated into eukaryotic cells including animal cells, fungal cells or plant cells. Preferred animal cells can be vertebrate or invertebrate. Preferred vertebrate cells include cells from mammals including rodents such as rats, mice, and hamsters; ungulates, such as cows, goats or sheep; and swine. Preferred vertebrate cells also include cells from human tissues and human stem cells. Target cells types include lymphocytes, hepatocytes, neural cells, muscle cells, blood cells, embryonic stem cells, somatic stem cells, hematopoietic cells, embryos, zygotes and sperm cells (some of which are open to be manipulated in an in vitro setting). Preferred cells can be pluripotent cells (cells whose descendants can differentiate into several restricted cell types, such as hematopoietic stem cells or other stem cells) or totipotent cells (i.e., a cell whose descendants can become any cell type in an organism, e.g., embryonic stem cells). Preferred culture cells are Chinese hamster ovary (CHO) cells, Human embryonic kidney (HEK293) cells, Henrietta Lacks (HeLa) cells, PERC6 cells, NS0 cells, and A549 cells. Preferred fungal cells are yeast cells including Saccharomyces cerevisiae and Pichia pastoris. Preferred plant cells are algae, for example Chlorella, tobacco, maize and rice (Nishizawa-Yokoi et al (2014) Plant J. 77:454-63 “Precise marker excision system using an animal derived piggyBac transposon in plants”). Preferred procaryotic cells include E. Coli K12 and E. Coli B derived cells.

Preferred gene transfer systems comprise a transposon in combination with a corresponding transposase protein that transposases the transposon, or a nucleic acid that encodes the corresponding transposase protein and is expressible in the target cell.

A transposase protein can be introduced into a cell as a protein or as a nucleic acid encoding the transposase, for example as a ribonucleic acid, including mRNA or any polynucleotide recognized by the translational machinery of a cell; as DNA, e.g. as extrachromosomal DNA including episomal DNA; as plasmid DNA, or as viral nucleic acid. Furthermore, the nucleic acid encoding the transposase protein can be transfected into a cell as a nucleic acid vector such as a plasmid, or as a gene expression vector, including a viral vector. The nucleic acid can be circular or linear. DNA encoding the transposase protein can be stably inserted into the genome of the cell or into a vector for constitutive or inducible expression. Where the transposase protein is transfected into the cell or inserted into the vector as DNA, the transposase encoding sequence is preferably operably linked to a heterologous promoter. There are a variety of promoters that could be used including constitutive promoters, tissue-specific promoters, inducible promoters, and the like. All DNA or RNA sequences encoding PiggyBac transposase proteins are expressly contemplated. Alternatively, the transposase may be introduced into the cell directly as protein, for example using cell-penetrating peptides (Ramsey et al., Pharmacol Ther, 2015) (Astolfo et al., Cell, 2015); or electroporation (Morgan et al., Methods in Mol Bio, 1995).

Natural DNA transposons undergo a ‘cut and paste’ system of replication in which the transposon is excised from a first DNA molecule and inserted into a second DNA molecule. DNA transposons are characterized by inverted terminal repeats (ITRs) and are mobilized by an element-encoded transposase. The piggyBac transposon/transposase system is particularly useful because of the frequency with which it is integrated (Fraser et al., Insect Transgenesis, CRC Press, 2001) and references therein.

Many sequences with sequence similarity to the piggyBac transposase from Trichoplusia ni have been found in the genomes of phylogenetically distinct species from fungi to mammals, but very few have been shown to possess transposase activity (Wu et al., Genetica, 2011) and references therein.

Transposases may also be fused to other protein functional domains. Such protein functional domains can include DNA binding domains, flexible hinge regions that can facilitate one or more domain fusions, and combinations thereof. Fusions can be made either to the N-terminus, C-terminus, or internal regions of the transposase protein so long as transposase activity is retained. DNA binding domains used can include a helix-turn-helix domain, Zn-finger domain, a leucine zipper domain, or a helix-loop-helix domain. Flexible hinge regions used can include glycine/serine linkers and variants thereof.

In particular, the object underlying the present invention is solved by the addition of a polypeptide with targeted DNA binding domains as described below. Surprisingly, the present inventors identified that addition of certain DNA binding elements to a transposase conferred not only improvements in transgene productivity over the wild-type sequence, but also improved stability of transgene expression. It appears that the present invention targets transgenes to regions of high transcriptional activity and high genetic stability. Surprisingly, the present invention increases transcription rates and transcriptional stability of the transgene while targeting noncoding sequences. This is a surprising, novel, and useful aspect of the invention as previous examples of semi-targeted gene integration that result in high transcription of the transgene have relied on targeting actively transcribed genes. Such genetic targeting systems have an inherently high risk of oncogenicity due to high likelihood of genetic disruption or improper genetic activation. For example, gamma retrovirus and lentivirus are retroviruses which transfer genes into a host cell via a type I retrotransposon, murine leukemia virus (MLV), a gamma retrovirus commonly used for gene integration in vitro has been shown to be a potently oncogenic due to its high preference for genomic integration near the start of transcriptional units (Wu et al., Science, 2003). Similarly, lentivirus has been shown to be oncogenic due to a preference to insert into actively transcribed genes within the transcriptional unit, anywhere downstream of the start site. MLV vectors and similar viruses/viral vectors, upon integration can enhance expression of the proceeding genetic sequences while HIV and similar viruses/viral vectors can reduce expression of the gene they disrupt upon integration. Both functions have been shown to be oncogenic in vitro and clinically (Modlich et al., J Clin Invest. 2009), (Modlich et al., Leukemia, 2008), and (Zhou et al., Mol Ther., 2016).

The ability to preferentially integrate a gene into a locus in the genome to increase genetic stability and transgene transcription, while avoiding deleterious and oncogenic disruptions is a so far unmet need in the field of genetic engineering. Such technologies would be particularly advantageous in the development of cell therapy products where risks of oncogenic transformation of the engineered cell must be completely mitigated yet high and stable expression of the transgene is also a clinical necessity.

The present inventors made the surprising discovery that fusion of a GC-rich DNA binding domain to a transposase can improve the stability and expression of transgenes integrated by transposases. The finding that targeting GC-rich sequences for transposition increases both transcript stability and expression was particularly surprising given the well-known role of CpG islands in epigenetic silencing and the role of GC-rich DNA segments in DNA heterochromatinization in eukaryotic genomes (H. Tamaru et al., Genes & Dev, 2010), (D. Takai et al., PNAS, 2002), (P. Jones et al., Proc Natl Acad, 2002).

Thus, in a first aspect, the present invention relates to a composition comprising a transposase, a fragment, or a derivative thereof having DNA transpositional activity and at least one GC-Rich DNA Binding Domain (GRDBD). Said composition can favorably enhance insertion site selection when compared to the native transposase.

The GRDBD may be a polypeptide sequence, an RNA sequence such as an aptamer or guide RNA sequence, or a small molecule compound. In one preferred embodiment the GRDBD and the transposase can be synthesized in cells by transfection of a nucleic acid. In one preferred embodiment, the GRDBD is either an RNA or polypeptide.

In one preferred embodiment the GRDBD is an RNA sequence. In one embodiment, the RNA sequence may be a guide RNA which targets a sequence comprising >80% GC in a 5 bp window. Said guide RNA may by coupled to the transposase domain through a covalently or noncovalently linked additional RNA sequence such as a tracrRNA or aptamer sequence. This additional RNA sequence may then mediate binding to an engineered transposase sequence containing a complementary binding polypeptide, small molecule, or other biomolecule sequence such that the transposase and the guide RNA are able to form a link.

In one preferred embodiment the GRDBD is a polypeptide sequence. The polypeptide may be a molecule comprising a transposase and at least one heterologous GRDBD which can either be translated as a single chain polypeptide from the same nucleic acid molecule, e.g. mRNA molecule, or can be produced by separate translation of the transposase and the at least one heterologous GRDBD and subsequent coupling, e.g. by adhesion forces or chemically. In the first case, the at least one GRDBD is fused/attached to the transposase. In the second case, the at least one GRDBD is linked/coupled to the transposase. The preferred linkage is a covalent linkage. The polypeptide may be designated as recombinant/artificial polypeptide. Preferably, the polypeptide is a single chain polypeptide which may also be designated as hybrid polypeptide or fusion polypeptide.

In one embodiment, the at least one heterologous GRDBD is connected to the transposase. Preferably, the at least one heterologous GRDBD is connected to the transposase via a linker. The connection may be a linkage/coupling or a fusion/attachment. In particular, when the linker is present, the at least one GRDBD is linked/coupled or fused/attached to the transposase via the linker. If the polypeptide is produced as a single chain polypeptide (which may also be designated as a hybrid polypeptide or fusion polypeptide), the GRDBD is attached/fused to the transposase via the linker. If the polypeptide is produced by separate translation of the GRDBD and the transposase and subsequent coupling, e.g. by adhesion forces or chemically, the GRDBD is linked/coupled to the transposase via the linker. The preferred linkage is a covalent linkage.

In one preferred embodiment, the at least one heterologous GRDBD is connected to the N-terminus of the transposase, to the C-terminus of the transposase, or to the N-terminus and C-terminus of the transposase. Preferably, the at least one heterologous GRDBD is connected to the N-terminus of the transposase, to the C-terminus of the transposase, or to the N-terminus and C-terminus of the transposase via a linker.

In one preferred embodiment, the at least one heterologous GRDBD forms the N-terminus of the polypeptide, the C-terminus of the polypeptide, or the N-terminus and C-terminus of the polypeptide and is particularly coupled to the transposase via a linker.

The heterologous GRDBDs forming the N-terminus of the transposase/polypeptide and the C-terminus of the transposase/polypeptide may be identical or different. They may be coupled to the transposase/polypeptide via identical or different linkers.

As mentioned above, one or more linkers may be comprised in the polypeptide to connect the one or more DNA Binding Domain with the transposase. For example, one linker may be comprised to connect the N-terminus of the transposase with the GRDBD, one linker may be comprised to connect the C-terminus of the transposase with the GRDBD, or one linker may be comprised to connect the N-terminus of the transposase with a GRDBD and one another (identical or different) linker may be comprised to connect the C-terminus of the transposase with another (identical or different) GRDBD. Said linker may comprise at least 2, 3, 4, or 5 amino acids. Preferably, the linker is a flexible linker. More preferably, the linker is a glycine linker, a serine-glycine linker.

In one alternatively preferred embodiment, the GRDBD is coupled/connected to the transposase via a binding molecule/moiety (instead of a linker). The molecule/moiety binding the GRDBD is preferably connected to the N-terminus or C-terminus of the transposase. Said binding molecule/moiety interacts with the transposase as well as with the GRDBD.

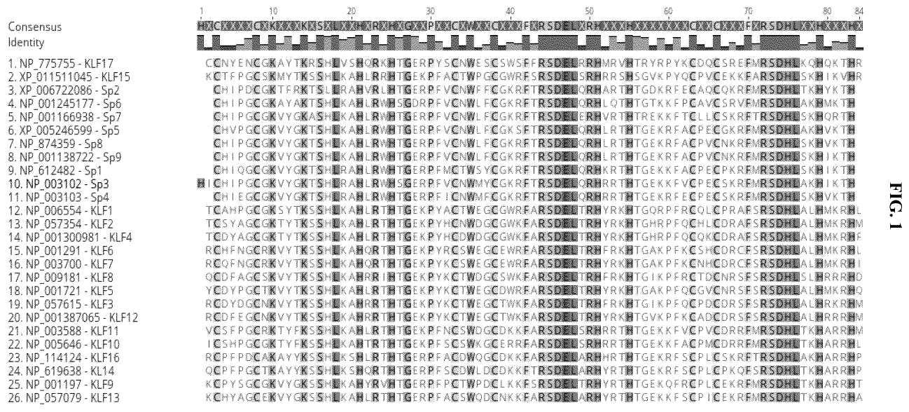

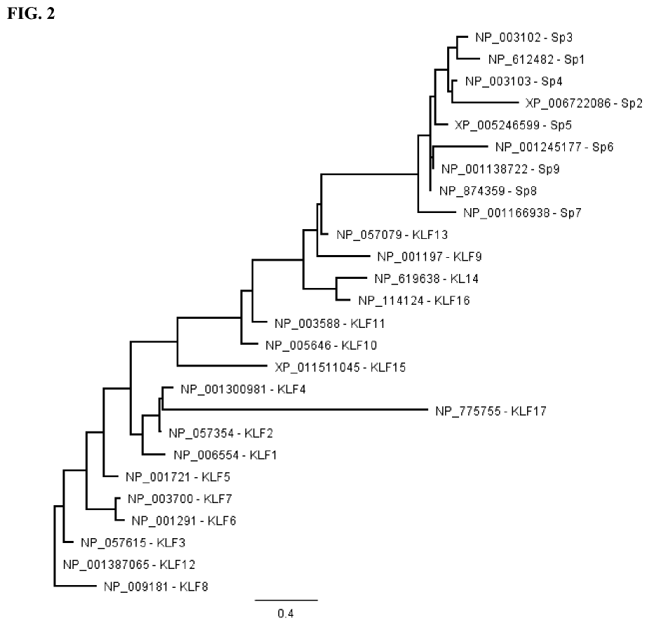

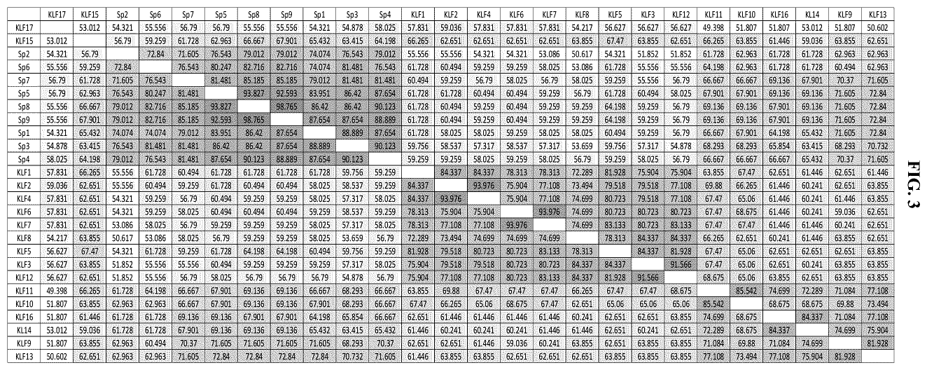

In one preferred embodiment, the at least one heterologous GRDBD is a GC-Rich Binding Protein (GRBP). Preferably, the at least one heterologous GRBP is a naturally occurring GRBP. The (naturally occurring) GC-Rich DNA binding domain may be an Sp/Klf family protein. Sp1-like proteins and Krüppel-like factors (KLFs) are highly related zinc-finger proteins that are important components of the eukaryotic cellular transcriptional machinery. By regulating the expression of a large number of genes that have GC-rich promoters, Sp1-like/KLF transcription regulators may take part in virtually all facets of cellular function. Most members of this family have been identified in mammals, with at least 21 Sp1-like/KLF proteins encoded in the human genome, Sp1-like/KLF proteins have highly conserved carboxy-terminal zinc-finger domains that function in DNA binding (J. Kaczynski et al., Gen Bio, 2003). The Sp family of transcription factors binds GC-rich DNA sequences. Evolutionarily, Sp1 and Sp3 represent the most recent duplication of the Sp family. Sp4 appears to be the most ancestral member. Sp1, Sp3, and Sp4 form a monophyletic group without Sp2. Sp2 is the least similar of the Sp family and is more similar to the non-Sp transcription factors. Only two domains (zinc fingers and B domain) share similarity outside the Sp family. The zinc fingers are homologous to other GC-binding domains, yet the B domain is homologous to protein-protein interacting domains in the CCAAT-binding/NF-Y transcription factor families. The Sp1-9 transcription factors are listed as SEQ ID NO: 14-22. The KLF1-17 transcription factors are listed as SEQ ID NO: 23-39. The zinc finger domains of the Sp1-9 transcription factors are listed as SEQ ID NO: 40-48 and the zinc finger domains of KLF1-17 are listed as SEQ ID NO: 49-65. An alignment of the human Sp and KLF zinc finger domains is shown in FIG. 1 and a corresponding distance tree is shown in FIG. 2. Further, FIG. 3 shows a distance matrix for the 26 sequences. Clearly, a domain with at least 50% sequence identity to any of SEQ ID NO: 40-65 may be a functionally equivalent GC-rich binding zinc finger sequence. Thus, in a preferred embodiment, the GRDBD is a protein having at least 50% sequence identity to SEQ ID Nos 40-65. Preferably the zinc finger is derived from the Sp1 transcription factor, e.g. having an amino acid sequence according to SEQ ID NO: 14 or SEQ ID NO: 40 or an amino acid sequence having at least 70%, e.g. 70, 75, 80, 85, 90, 95, 96, 97, 98, or 99% identity to SEQ ID NO:14 or SEQ ID NO: 40.

In one embodiment the GRBP is any other GC-rich binding protein that is not homologous to the Sp/Klf transcription factor family. These include but are not limited to vasculin (GPBP1), vasculin like-protein 1 (GPBPIL1), GCFC2, PAXBP1, ZNF281, and IRF4, listed as SEQ ID NO: 66-71 respectively. Preferably the GRBP is derived from the GPBP1, e.g. having an amino acid sequence according to SEQ ID NO: 66 or an amino acid sequence having at least 70%, e.g. 70, 75, 80, 85, 90, 95, 96, 97, 98, or 99%, sequence identify thereto.

In one embodiment, a GRBP is comprised at the N-terminus of the transposase and is particularly coupled to the transposase via a linker. In one embodiment, a GRBP is comprised at the C-terminus of the transposase and is particularly coupled to the transposase via a linker. In one embodiment, a GRBP is comprised at the N-terminus and at the C-terminus of the transposase, both are particularly coupled to the transposase via a linker.

The nucleotide sequences and the corresponding amino acid sequences of preferred polypeptides comprising a transposase and at least one heterologous GRBP are listed under SEQ ID NO: 62 for Sp1-PB_CTVar9, SEQ ID NO: 63 for Sp1_ZFN-PB_CTVar9, under SEQ ID NO: 64 for GPBP1-PB_CTVar9. The wildtype PB transposase is listed under SEQ ID NO: 1. The nucleotide sequence corresponding to the wildtype Teratorn transposase is listed under SEQ ID NO: 2. A preferred hyperactive PiggyBac transposase variant, PB_CTVar9 is listed under SEQ ID NO: 13. The nucleotide sequences and the corresponding amino acid sequences of preferred polypeptides comprising a transposase and at least one heterologous GRBP are listed under SEQ ID NO: 77 for Sp1-Teratorn, SEQ ID NO: 78 for Sp1_ZFN-Teratorn, and SEQ ID NO: 79 for Gpbp1-Teratorn. Variants (on the nucleotide sequence as well as amino acid level) of the above-mentioned sequences are also encompassed. Said variants have at least 90%, e.g. 90, 91, 92, 93, 94, 95, 96, 97, 98, or 99%, sequence identity to the above-mentioned sequences. The variants are functionally active variants or code for functionally active variants. Functionally active variants are still able to detect and bind GC-rich DNA and are still able to excise and insert transposable elements.

The present inventors made the surprising discovery that fusion of a multimerization domain to a transposase can improve the expression of transgenes integrated by transposases. Surprisingly, the present inventors identified that addition of a dimerization domain has no positive effect on transposition activity while addition of a tetramerization domain improves transposition frequency.

Thus, in a first aspect, the present invention relates to a composition comprising a transposase, a fragment, or a derivative thereof having DNA transpositional activity and at least one tetramerization domain. Said composition can favorably enhance transposition rate when compared to a native transposase.

In one preferred embodiment the tetramerization is a polypeptide sequence. The polypeptide may be a molecule comprising a transposase and at least one heterologous tetramerization domain which can either be translated as a single chain polypeptide from the same nucleic acid molecule, e.g. mRNA molecule, or can be produced by separate translation of the transposase and the at least one heterologous tetramerization domain and subsequent coupling, e.g. by adhesion forces or chemically. In the first case, the at least one tetramerization domain is fused/attached to the transposase. In the second case, the at least one tetramerization domain is linked/coupled to the transposase. The preferred linkage is a covalent linkage. The polypeptide may be designated as recombinant/artificial polypeptide. Preferably, the polypeptide is a single chain polypeptide which may also be designated as hybrid polypeptide or fusion polypeptide.

In one embodiment, the at least one heterologous tetramerization domain is connected to the transposase. Preferably, the at least one heterologous tetramerization domain is connected to the transposase via a linker. The connection may be a linkage/coupling or a fusion/attachment. In particular, when the linker is present, the at least one tetramerization domain is linked/coupled or fused/attached to the transposase via the linker. If the polypeptide is produced as a single chain polypeptide (which may also be designated as a hybrid polypeptide or fusion polypeptide), the tetramerization domain is attached/fused to the transposase via the linker. If the polypeptide is produced by separate translation of the tetramerization domain and the transposase and subsequent coupling, e.g. by adhesion forces or chemically, the tetramerization domain is linked/coupled to the transposase via the linker. The preferred linkage is a covalent linkage.

In one preferred embodiment, the at least one heterologous tetramerization domain is connected to the N-terminus of the transposase, to the C-terminus of the transposase, or to the N-terminus and C-terminus of the transposase. Preferably, the at least one heterologous tetramerization domain is connected to the N-terminus of the transposase, to the C-terminus of the transposase, or to the N-terminus and C-terminus of the transposase via a linker.

In one preferred embodiment, the at least one heterologous tetramerization domain forms the N-terminus of the polypeptide, the C-terminus of the polypeptide, or the N-terminus and C-terminus of the polypeptide and is particularly coupled to the transposase via a linker.

The heterologous tetramerization domain forming the N-terminus of the transposase/polypeptide and the C-terminus of the transposase/polypeptide may be identical or different. They may be coupled to the transposase/polypeptide via identical or different linkers.

As mentioned above, one or more linkers may be comprised in the polypeptide to connect the one or more multimerization domains with the transposase. For example, one linker may be comprised to connect the N-terminus of the transposase with the tetramerization domain or dimerization domain, one linker may be comprised to connect the C-terminus of the transposase with the tetramerization domain or dimerization domain, or one linker may be comprised to connect the N-terminus of the transposase with a tetramerization domain or dimerization domain and one another (identical or different) linker may be comprised to connect the C-terminus of the transposase with another (identical or different) tetramerization domain or dimerization domain. Said linker may comprise at least 2, 3, 4, or 5 amino acids. Preferably, the linker is a flexible linker. More preferably, the linker is a glycine linker, a serine-glycine linker.

In one alternatively preferred embodiment, the tetramerization domain is coupled/connected to the transposase via a binding molecule/moiety (instead of a linker). The molecule/moiety binding the tetramerization domain is preferably connected to the N-terminus or C-terminus of the transposase. Said binding molecule/moiety interacts with the transposase as well as with the tetramerization domain.

In one preferred embodiment, the at least one heterologous multimerization domain is a dimerization domain. Preferably, the at least one heterologous multimerization domain is a naturally occurring multimerization domain. The (naturally occurring) multimerization domain may be a leucine/isoleucine zipper family. Leucine zipper and isoleucine zipper families are highly structurally related multimerization peptides that are important components of the eukaryotic cellular machinery. By regulating the activity of a large number of genes that depend on the functional formation of dimerized proteins, leucine zippers may take part in virtually all facets of cellular function. Vast differences in primary amino acid sequence are possible while maintaining tertiary and secondary structures of leucine zippers, thus convergent evolution has generated a vast array of naturally occurring leucine zippers with poor sequence homology. Additionally, completely synthetic leucine zippers have been developed with relative ease due to the simplicity of the prediction of the folding of these structures. Some exemplary leucine zipper sequences are provided in SEQ ID NO: 123, 126-132.

In one preferred embodiment, the at least one heterologous multimerization domain is a tetramerization domain. Preferably, the at least one heterologous multimerization domain is a naturally occurring tetramerization domain. The (naturally occurring) tetramerization domain may be a MADS transcription factor family. The A-E class MADS genes encode TFs that act in a combinatorial manner in an intricate protein-protein interaction network, forming both dimeric and tetrameric complexes and driving different developmental programs. MADS TFs bind DNA as dimers through the MADS DNA-binding domain, a domain that is conserved across all eukaryotes. The addition of an alpha-helical keratin-like or ‘K’ domain allows plant MADS TFs to tetramerize, with the structure of the K domain of SEP3 recently characterized and the tetramerization determinants mapped at the amino acid level (Puranik S. et al, Plant Cell. 2014). A nonlimiting exemplary tetramerization sequence is provided in SEQ ID NO: 122 for the SEP3 tetramerization domain from Arabidopsis thaliana.

The amino acid sequences of preferred polypeptides comprising a transposase and at least one heterologous multimerization domain are listed under SEQ ID NO: 114 for PB-CTVar14+bZIP_GCN4, SEQ ID NO: 116 for PB-CTVar14+Gpbp1+Tetramerizer_SEP3, under SEQ ID NO: 119 for PB-CTVar14+Sp1_ZFN+Tetramerizer_SEP3, under SEQ ID NO:120 for PB-CTVar14+Sp1+Tetramerizer_SEP3, under SEQ ID NO:121 for PB-CTVar14+Tetramerizer. The wildtype PB transposase is listed under SEQ ID NO: 1. The nucleotide sequence corresponding to the wildtype Teratorn transposase is listed under SEQ ID NO:2. A variety of preferred hyperactive transposases are listed under SEQ ID NO:5-13, 102-113. Variants (on the nucleotide sequence as well as amino acid level) of the above-mentioned sequences are also encompassed. Said variants have at least 90%, e.g. 90, 91, 92, 93, 94, 95, 96, 97, 98, or 99%, sequence identity to the above-mentioned sequences. The variants are functionally active variants or code for functionally active variants. Functionally active variants are still able to form tetramers and are still able to excise and insert transposable elements.

The transposase may be a transposase of class I (retrotransposase) or a transposase of class II (DNA transposase). In case of a transposase of class I, the transposase may also be designated as integrase. In one preferred embodiment, the transposase is a class II transposase (DNA transposase). In one more preferred embodiment, the transposase is a PiggyBac transposase, a sleeping beauty transposase, a Tol2 transposase, a Helraiser transposase, a TcBuster transposase (SEQ ID NO:83), a Hermes transposase (SEQ ID NO:82), or a Teratorn transposase. Preferably, the PiggyBac transposase is a wild-type PiggyBac transposase, a hyperactive PiggyBac transposase, a wild-type PiggyBac-like transposase, or a hyperactive PiggyBac-like transposase. The wild-type PiggyBac transposase has more preferably an amino acid sequence according to SEQ ID NO: 1 or an amino acid sequence having at least 90%, e.g. at least 90, 91, 92, 93, 94, 95, 96, 97, 98, or 99%, sequence identity thereto. The wild-type PiggyBac transposase variants are functionally active variants, i.e. they are still able to function as transposases (excision as well as integration of polynucleotides). The PiggyBac-like transposase is more preferably selected from the group consisting of PiggyBat, PiggyBac-like transposase from Xenopus tropicalis, PiggyBac-like transposase from Bombyx mori, a PiggyBac-like transposase from Bactrocera dorsalis. The PiggyBac-like transposase sequences has more preferably an amino acid sequence according to SEQ ID NO:101 or an amino acid sequence having at least 90%, e.g. at least 90, 91, 92, 93, 94, 95, 96, 97, 98, or 99%, sequence identity thereto.

Preferably, the PiggyBac transposase is a hyperactive PiggyBac transposase or a hyperactive PiggyBac-like transposase. The hyperactive PiggyBac transposase has more preferably an amino acid sequence according to any of SEQ ID NO: 5-13. The hyperactive PiggyBac-like transposase has an amino acid sequence according to any of SEQ ID NO: 102-113. Preferably, the hyperactive PiggyBac transposase or PiggyBac-like transposase has at least two of the following mutations when number according to alignment to SEQ ID NO: 101; W149H, FR167-169WK, M281Q, G315Q, S372K, Q440K, N462R, VS488-489NP, MRN502-504LKE, TSDDSTE542-548AS. More preferably, the hyperactive PiggyBac transposase or PiggyBac-like transposase has at least the following mutations Q440K and MRN502-504LKE.

In one further preferred embodiment, the polypeptide further comprises at least one heterologous DNA binding domain (e.g. at least 1 or 2 DNA binding domain(s)).

In a second aspect, the present invention relates to a polynucleotide encoding the polypeptide according to the first aspect. Said polynucleotide is preferably DNA or RNA such as mRNA.

In a third aspect, the present invention relates to a vector comprising the polynucleotide according to the second aspect. The terms “vector” and “plasmid” can interchangeable be used herein. The vector may be a viral or non-viral vector. Preferably, the vector is an expression vector. The expression of the polynucleotide encoding the polypeptide according to the first aspect is preferably controlled by expression control sequences. Expression control sequences may be sequences which control the transcription, e.g. promoters, enhancers, UCOE or MAR elements, polyadenylation signals, post-transcriptionally active elements, e.g. RNA stabilising elements, RNA transport elements and translation enhancers. Said expression control sequences are known to the skilled person. For example, as promoters, CMV or PGK promoters may be used.

In a fourth aspect, the present invention relates to a method for producing a cell, in particular transgenic cell, comprising the steps of: (i) providing a cell, and (ii) introducing a transposable element comprising at least one polynucleotide of interest, and a polypeptide according to the first aspect, a polynucleotide according to the second aspect, or a vector according to the third aspect into the cell, thereby producing/obtaining the cell, in particular transgenic cell.

The method may be an in vitro or in vivo method. Naturally, a transposable element includes a polynucleotide encoding a functional transposase that catalyzes excision and insertion. The transposable element referred to in step (ii) of the above-mentioned method is, however, devoid of a polynucleotide encoding a functional transposase. The transposable element does not comprise the complete sequence encoding a functional, preferably a naturally occurring, transposase. Preferably, the complete sequence encoding a functional, preferably a naturally occurring, transposase or a portion thereof, is deleted from the transposable element. Instead of a polynucleotide encoding a functional transposase, at least one polynucleotide of interest, e.g. at least one exogenous/heterologous polynucleotide, is part of the transposable element described above. Thus, said transposable element may also be designated as recombinant/artificial transposable element.

The transposase or a fragment or a derivative thereof having transposase function connected to at least one GC-rich DNA Binding Domain (GRDBD) is provided in step (ii) of the above-mentioned method in trans, e.g. as a polypeptide according to the first aspect, as a polynucleotide according to the second aspect, or comprised in a vector according to the third aspect.

The introduction of the transposable element comprising at least one polynucleotide of interest may take place via electroporation, transfection, injection, lipofection, or (viral) infection. The transposable element comprising at least one polynucleotide of interest may be introduced transiently or stably into the cell. In the first case, the transposable element comprising at least one polynucleotide of interest is introduced as extrachromosomal element, e.g. as linear DNA molecule, plasmid DNA, episomal DNA, viral DNA, or viral RNA. In the second case, the transposable element comprising at least one polynucleotide of interest is stably introduced/inserted into the genome of the cell. Preferably, the transposable element comprising at least one polynucleotide of interest is transiently introduced into the cell. More preferably, the transposable element comprising at least one polynucleotide of interest is comprised in a vector. The person skilled in the art is well informed about molecular biological techniques, such as microinjection, electroporation or lipofection, for introducing the transposable element into a cell and knows how to perform these techniques.

The introduction of the polypeptide according to the first aspect, the polynucleotide according to the second aspect, or the vector according to the third aspect may also take place via electroporation, transfection, injection, lipofection, and/or (viral) infection.

If a polynucleotide is introduced into the cell, the polynucleotide is subsequently transcribed and translated into the polypeptide in the cell. If a vector comprising the polynucleotide is introduced into the cell, the polynucleotide is subsequently transcribed from the vector and translated into the polypeptide in the cell. The polynucleotide may be DNA or RNA such as mRNA. Also, viral DNA or RNA may be introduced. The polynucleotide may be introduced transiently or stably into the cell. In the first case, the polynucleotide is introduced as extrachromosomal polynucleotide, e.g. as linear DNA molecule, circular DNA molecule, plasmid DNA, viral DNA, in vitro synthesised/transcribed RNA, or viral RNA. In the second case, the polynucleotide is stably introduced/inserted into the genome of the cell. Preferably, the polynucleotide is transiently introduced into the cell. More preferably, the polynucleotide is comprised in a vector, in particular in an expression vector. The viral DNA or RNA sequences may also be introduced as part of a vector or in form of a vector. It is particularly preferred that the polynucleotide is operably linked to a heterologous promoter allowing the transcription of the transposase, or a fragment or a derivative thereof having transposase function and the at least one GC-rich DNA binding Domain within the cell or from a vector, e.g. expression vector or a vector used for in vitro transcription, comprised in the cell.

The person skilled in the art is well informed about molecular biological techniques, such as microinjection, electroporation or lipofection, for introducing polypeptides or nucleic acid sequences encoding polypeptides into a cell and knows how to perform these techniques.

In one preferred embodiment, the transposable element comprising at least one polynucleotide of interest is comprised in/part of a polynucleotide molecule, preferably a vector. In this case, the polynucleotide according to the second aspect is also preferably comprised in/part of a (different) polynucleotide molecule, preferably a (different) vector. Thus, it is preferred that the polynucleotide according to the second aspect and the transposable element are on separate polynucleotide molecules, preferably vectors. This allows the adaptation of transposase and transposable element plasmid amounts to achieve a few or as many integrations per cell as desired.

In one alternatively preferred embodiment, the transposable element comprising at least one polynucleotide of interest and the polynucleotide according to the second aspect are comprised in/part of a (the same) polynucleotide molecule, preferably a vector. In this case, it is preferred that the polynucleotide according to the second aspect is located external to the region of the at least one polynucleotide of interest. Preferably, said polynucleotide is operably linked to a heterologous promoter allowing the transcription of the transposase, or a fragment or a derivative thereof having transposase function and the at least one DNA Binding Domain from the polynucleotide molecule, preferably vector.

The transposable element referred to in step (ii) of the above-mentioned method retains sequences that are required for mobilization by the transposase provided in trans. These are the repetitive sequences at each end of the transposable element containing the binding sites for the transposase allowing the excision from the genome. Thus, in one embodiment, the transposable element comprises terminal repeats (TRs). In one further embodiment, the at least one polynucleotide of interest is flanked by TRs. For example, the transposable element referred to in step (ii) of the above-mentioned method comprises a first transposable element-specific terminal repeat and a second transposable element-specific terminal repeat downstream of the first transposable element-specific terminal repeat. The at least one polynucleotide of interest is located between the first transposable element-specific terminal repeat and the second transposable element-specific terminal repeat. Preferably, the terminal repeats are inverted terminal repeats (ITRs) or long terminal repeats (LTRs). In this respect, it should be noted that the transposase provided in trans is specific for the transposable element. In other words, the transposable element is specifically recognized by the transposase. A transposase of class II (DNA transposase), for example, recognizes a TA dinucleotide at each end of the transposable element, particularly within the repetitive sequences/terminal repeats of the transposable element. It also recognizes a TA dinucleotide in the target sequence.

As mentioned above, the transposable element comprising at least one polynucleotide of interest and the polynucleotide according to the second aspect are comprised in/part of a (the same) polynucleotide molecule, preferably a vector. In this case, it is preferred that the polynucleotide according to the second aspect is located external to the region of the at least one polynucleotide of interest. It is particularly preferred that the polynucleotide according to the second aspect is located outside of the terminal repeats, e.g. inverted terminal repeats (ITRs) or long terminal repeats (LTR), flanking the at least one polynucleotide of interest.

The transposable element may be derived from a prokaryotic or an eukaryotic transposable element, wherein the latter is preferred.

The transposable element may be a Class II or a DNA/DNA-based transposable element. The DNA/DNA-based transposable element comprises inverted terminal repeats (ITRs). It is recognized by a transposase of class II (DNA transposase). The transposable element may also be a Class I or a retrotransposable element. The retrotransposable element may be a long terminal repeat (LTR) retrotransposable element. The LTR retrotransposable element comprises long terminal repeats (LTRs). It is recognized by a transposase of class I (retrotransposase). Said transposase may also be designated as integrase.

As mentioned above, class II or DNA-based transposable elements contain inverted terminal repeats (ITRs) at either end. Conservative DNA-based transposable elements move by a cut-and-paste mechanism. This requires a transposase, inverted repeats at the ends of the transposable element and a target sequence on the new host DNA molecule. The transposase is provided in the above mentioned method in trans. It catalysis the excision of the transposable element from the current location and the integration of the excised transposable element into the genome of a cell. In the cut-and-paste mechanism, the transposase specifically binds to the inverted terminal repeats of the transposable element and cuts the transposable element out of the current location, e.g. vector. The transposase then locates the transposable element, cuts the target DNA backbone and then inserts the transposable element. Usually, two transposase monomers are involved in the excision of the transposable element, one transposase monomer at each end of the transposable element. Finally, the transposase dimer in complex with the excised transposable element reintegrates the transposable element in the DNA of a cell.

In one preferred embodiment, the transposable element is a class II or DNA-based transposable element. In one more preferred embodiment, the transposable element is a PiggyBac transposable element, a sleeping beauty transposable element, a Hermes transposable element, a Helraiser transposable element, a TcBuster transposable element, a Teratorn transposable element or a Tol2 transposable element. Preferably, the PiggyBac transposable element is a wild-type PiggyBac transposable element, a hyperactive PiggyBac transposable element, a wild-type PiggyBac-like transposable element, or a hyperactive PiggyBac-like transposable element. The PiggyBac-like transposable element is more preferably selected from the group consisting of a PiggyBat transposable element, a PiggyBac-like transposable element from Xenopus tropicalis, and a PiggyBac-like transposable element from Bombyx mori. The PiggyBac DNA transposable element is, for example, used technologically and commercially in genetic engineering by virtue of its property to efficiently transpose between vectors and chromosomes.

In one further preferred embodiment, the transposon-specific inverted terminal repeats comprise the PiggyBac minimal ITR. In one more preferred embodiment, the first transposon-specific inverted terminal repeat comprises the sequence according to SEQ ID NO: 84 or a sequence having at least 90%, e.g. at least 90, 91, 92, 93, 94, 95, 96, 97, 98, or 99%, sequence identity thereto, and/or the second transposon-specific inverted terminal repeat comprises the sequence according to SEQ ID NO: 85 or a sequence having at least 90%, e.g. at least 90, 91, 92, 93, 94, 95, 96, 97, 98, or 99%, sequence identity thereto. The PiggyBac minimal ITR variants are functionally active variants, i.e. they can still be recognized by a transposase specific for the PiggyBac minimal ITR.

In one further preferred embodiment, the transposon-specific inverted terminal repeats comprise the Teratorn ITR. In one more preferred embodiment, the first transposon-specific inverted terminal repeat comprises the sequence according to SEQ ID NO: 86 or a sequence having at least 90%, e.g. at least 90, 91, 92, 93, 94, 95, 96, 97, 98, or 99%, sequence identity thereto, and/or the second transposon-specific inverted terminal repeat comprises the sequence according to SEQ ID NO: 87 or a sequence having at least 90%, e.g. at least 90, 91, 92, 93, 94, 95, 96, 97, 98, or 99%, sequence identity thereto.

In one further preferred embodiment, the transposon-specific inverted terminal repeats comprise the Hermes ITR. In one more preferred embodiment, the first transposon-specific inverted terminal repeat comprises the sequence according to SEQ ID NO: 88 or a sequence having at least 90%, e.g. at least 90, 91, 92, 93, 94, 95, 96, 97, 98, or 99%, sequence identity thereto, and/or the second transposon-specific inverted terminal repeat comprises the sequence according to SEQ ID NO: 89 or a sequence having at least 90%, e.g. at least 90, 91, 92, 93, 94, 95, 96, 97, 98, or 99%, sequence identity thereto.

In one further preferred embodiment, the transposon-specific inverted terminal repeats comprise the TcBuster minimal ITR. In one more preferred embodiment, the first transposon-specific inverted terminal repeat comprises the sequence according to SEQ ID NO: 90 or a sequence having at least 90%, e.g. at least 90, 91, 92, 93, 94, 95, 96, 97, 98, or 99%, sequence identity thereto, and/or the second transposon-specific inverted terminal repeat comprises the sequence according to SEQ ID NO: 91 or a sequence having at least 90%, e.g. at least 90, 91, 92, 93, 94, 95, 96, 97, 98, or 99%, sequence identity thereto. The TcBuster minimal ITR variants are functionally active variants, i.e. they can still be recognized by a transposase specific for the TcBuster minimal ITR.

In one further preferred embodiment, the transposon-specific inverted terminal repeats comprise the Sleeping Beauty ITR. In one more preferred embodiment, the first transposon-specific inverted terminal repeat comprises the sequence according to SEQ ID NO: 92 or a sequence having at least 90%, e.g. at least 90, 91, 92, 93, 94, 95, 96, 97, 98, or 99%, sequence identity thereto, and/or the second transposon-specific inverted terminal repeat comprises the sequence according to SEQ ID NO: 93 or a sequence having at least 90%, e.g. at least 90, 91, 92, 93, 94, 95, 96, 97, 98, or 99%, sequence identity thereto.

In one further preferred embodiment, the transposon-specific inverted terminal repeats comprise the Tol2 ITR. In one more preferred embodiment, the first transposon-specific inverted terminal repeat comprises the sequence according to SEQ ID NO: 94 or a sequence having at least 90%, e.g. at least 90, 91, 92, 93, 94, 95, 96, 97, 98, or 99%, sequence identity thereto, and/or the second transposon-specific inverted terminal repeat comprises the sequence according to SEQ ID NO: 95 or a sequence having at least 90%, e.g. at least 90, 91, 92, 93, 94, 95, 96, 97, 98, or 99%, sequence identity thereto.

The cell may be a prokaryotic or a eukaryotic cell. Preferably, the cell is a eukaryotic cell. More preferably, the eukaryotic cell is a vertebrate, a yeast, a fungus, or an insect cell. The vertebrate cell may be a mammalian, a fish, an amphibian, a reptilian cell or an avian cell. The avian cell may be a chicken, quail, goose, or duck cell such as a duck retina cell or duck somite cell. Even more preferably, the vertebrate cell is a mammalian cell. Most preferably, the mammalian cell is selected from the group consisting of a Chinese hamster ovary (CHO) cell (e.g. CHO-K1/CHO-S/CHO-DUXB11/CHO-DG44 cell), a human embryonic kidney (HEK293) cell, a HeLa cell, a A549 cell, a MRC5 cell, a WI38 cell, a BHK cell, and a Vero cell.

The cell may be an isolated cell (such as in a cell culture or in a cell line, e.g. stable cell line). The cell may also be a cell of a tissue outside of an organism. The transgenic cell may, however, subsequently be inserted into an organism. Insertion of the transgenic cell into the organisms may be effected by infusion or injection or further means well known to the person skilled in the art.

The cell may also be part of/comprised in an organism, e.g. eukaryotic multicellular organism. In this case, the insertion of a transposable element comprising at least one polynucleotide of interest, and a polypeptide according to the first aspect, a polynucleotide according to the second aspect, or a vector according to the third aspect is effected in vivo. In vivo polypeptide/polynucleotide/transposable element delivery can be accomplished by injection (either locally or systemically). The polynucleotide/transposable element can be, for example, in the form of naked DNA, DNA complexed with liposomes, PEI or other condensing agents, or can be incorporated into infectious particles (viruses or virus-like particles). Polynucleotide/transposable element delivery can also be done using electroporation or with gene guns or with aerosols.

Said organism may be a prokaryotic or an eukaryotic organism. Preferably, said organism is an eukaryotic organism. More preferably, said organism may be a fungus, an insect, or a vertebrate. The vertebrate may be a bird (e.g. a chicken, quail, goose, or duck), a canine, a mustela, a rodent (e.g. a mouse, rat or hamster), an ovine, a caprine, a pig, a bat (e.g. a megabat or microbat) or a human/non-human primate (e.g. a monkey or a great ape). Most preferably the organism is a mammal such as a mouse, a rat, a pig, or a human/non-human primate.

In one embodiment, the at least one polynucleotide of interest is selected from the group consisting of a polynucleotide encoding a polypeptide, a non-coding polynucleotide, a polynucleotide comprising a promoter sequence, a polynucleotide encoding a mRNA, a polynucleotide encoding a tag, and a viral polynucleotide.

The polypeptide encoded by the polynucleotide may be a therapeutically active polypeptide, e.g. an antibody, an antibody fragment, a monoclonal antibody, a virus protein, a virus protein fragment, an antigen, a hormone. The polypeptide may further be used for gene therapy, e.g. of monogenic diseases. In this case, the polynucleotide encoding the polypeptide is operably linked with a tissue-specific promoter. The polypeptide may also be used for cell therapy, in particularly ex vivo. The cells may be pluripotent stem cells (iPSC), human embryonic stem (hES) cells, human hematopoietic stem cells (HSCs), or human T lymphocytes.

The non-coding polynucleotide may be useful in the targeted disruption of a gene.

The polynucleotide comprising promoter sequences may allow the activation of gene expression if the transposon inserts close to an endogenous gene.

The polynucleotide may be transcribed into mRNA or a functional noncoding RNA e.g. a miRNAi or gRNA.

The polynucleotide may comprise a sequence tag to identify the insertion site of the transposable element.

The viral polynucleotide may be used for the production of biopharmaceutical products based on virus particles.

The transposable element and/or the vector comprising the transposable element may further comprise elements that enhance expression (e.g. nuclear export signals, promoters, introns, terminators, enhancers, elements that affect chromatin structure, RNA export elements, IRES elements, CHYSEL elements, and/or Kozak sequences), selectable marker (e.g. DHFR, puromycine, hygromycin, zeocin, blasticidin, and/or neomycin), markers for in vivo monitoring (e.g. GFP or beta-galactosidase), a restriction endonuclease recognition site (e.g. a site for insertion of an exogenous nucleotide sequence such as a multiple cloning site), a recombinase recognition site (e.g. LoxP (recognized by Cre), FRT (recognized by Flp), or AttB/AttP (recognized by PhiC31)), insulators (e.g. MARs or UCOEs), viral replication sequences (e.g. SV40 ori).

In the above-described method, not only one but also more than one transposable element may be inserted into the cell. The transposable elements may differ from each other, e.g. as they comprise different polynucleotides of interest. This is specifically desired in cases were two ORFs encoding antibody heavy chains (HC) or antibody light chains (LC) have to be introduced into the cell. In this case, the two or more ORFs are comprised in the same or on separate transposable elements, preferably on separate transposable elements.

In the fifth aspect, the present invention relates to a cell, in particular transgenic cell, obtainable/producible by the method of the fourth aspect.

In a sixth aspect, the present invention relates to the use of a cell, in particular transgenic cell, of the fifth aspect for the production of a protein or virus. The proteins may be therapeutic proteins. The virus may be a vector (viral vector).

In a seventh aspect, the prevent invention relates to a kit comprising (i) a transposable element comprising a cloning site for inserting at least one polynucleotide of interest, and (ii) a polypeptide according to the first aspect, a polynucleotide according to the second aspect, a vector according to the third aspect, or at least one heterologous GRDBD and a polypeptide comprising a transposase or a fragment or a derivative thereof having transposase function.

The transposable element provided with the kit/comprised in the kit is devoid of a polynucleotide encoding a functional transposase. The transposable element does not comprise the complete sequence encoding a functional, preferably a naturally occurring, transposase. Preferably, the complete sequence encoding a functional, preferably a naturally occurring, transposase or a portion thereof, is deleted from the transposable element. Instead of a polynucleotide encoding a functional transposase, the transposable element comprises a cloning site (in particular at least one cloning site) for inserting at least one polynucleotide of interest. The type of the polynucleotide of interest which is finally introduced into the transposable element depends on the end user. The transposable element may be a recombinant, an artificial, and/or a heterologous transposable element.

The transposase is an independent or a distinct component of the kit. It is provided with the kit/comprised in the kit connected to a heterologous GC Rich DNA Binding Domain (GRDBD) as a polypeptide according to the first aspect, as a polynucleotide according to the second aspect, or comprised in a vector according to the third aspect (see item (ii)).

In an alternative, a polypeptide comprising a transposase or a fragment, or a derivative thereof having transposase function is provided with the kit/comprised in the kit without being connected to a GC Rich DNA Binding Domain (GRDBD), in particular GC Rich Binding Protein (GRBP). In this specific case, the polypeptide comprising a transposase or a fragment, or a derivative thereof having transposase function and the GRDBD, in particular GRBP, is provided with the kit/comprised in the kit as independent or distinct components. Preferably, the GRDBD, in particular GRBP, is associated with a binding molecule/moiety which is-after introduction into a cell-able to bind the transposase (e.g. via the N-terminus or C-terminus) forming a transposase, binding molecule/moiety and GRDBD, in particular GRBP, complex. This, of course, requires that the polypeptide comprising a transposase, or a fragment, or a derivative thereof having transposase function comprises a binding domain allowing the binding molecule/moiety associated with the GRDBD, in particular GRBP, to bind. This binding domain is preferably a protein binding domain. Alternatively, the GRDBD, in particular GRBP, is associated with a binding molecule/moiety which is-after introduction into a cell-able to bind the transposable element. This, of course, requires that the transposable element comprises a binding domain allowing the binding molecule/moiety associated with the GRDBD, in particular GRBP, to bind. This binding domain is preferably a DNA binding domain. The polypeptide comprising a transposase or a fragment or a derivative thereof having transposase function may be a recombinant, an artificial, and/or a heterologous polypeptide.

The transposable element may be provided with the kit/comprised in the kit as a linear DNA molecule, plasmid DNA, episomal DNA, viral DNA, or viral RNA. It is preferred that the transposable element comprises a heterologous promoter which allows, after integration of the at least one polynucleotide of interest into the cloning site, the transcription of the at least one polynucleotide of interest. Preferably, the transposable element is comprised in a vector.

The polynucleotide according to the second aspect may also be provided with the kit/comprised in the kit as a linear DNA molecule, a circular DNA molecule, plasmid DNA, viral DNA, in vitro synthesized/transcribed RNA or viral RNA. It is preferred that the polynucleotide is operably linked to a heterologous promoter allowing the transcription of the transposase, or a fragment or a derivative thereof having transposase function and the at least one GRDBD. Preferably, the polynucleotide is comprised in a vector, in particular an expression vector or a vector for in vitro transcription.

The transposable element and the polynucleotide according to the second aspect may be part of different vectors. This allows the adaptation of transposase and transposable element plasmid amounts to achieve a few or as many integrations peer cell as desired.

The transposable element and the polynucleotide according to the second aspect may also be part of the same vector. In this case, it is preferred that the polynucleotide is located external to the cloning site for inserting at least one polynucleotide of interest.

The transposable element provided with the kit/comprised in the kit retains sequences that are required for mobilization by the transposase provided in trans. These are the repetitive sequences at each end of the transposable element containing the binding sites for the transposase allowing the excision from the genome. Thus, in one embodiment, the transposable element comprises terminal repeats (TRs). In one further embodiment, the at least one polynucleotide of interest is flanked by TRs. For example, the transposable element referred to in step (ii) of the above mentioned method comprises a first transposable element-specific terminal repeat and a second transposable element-specific terminal repeat downstream of the first transposable element-specific terminal repeat. The cloning site for inserting at least one polynucleotide of interest is located between the first transposable element-specific terminal repeat and the second transposable element-specific terminal repeat. Preferably, the terminal repeats are inverted terminal repeats (ITRs or TIRs) or long terminal repeats (LTRs). In this respect, it should be noted that the transposase provided with the kit/comprised in the kit is specific for the transposable element. In other words, the transposable element can specifically be recognized by the transposase. A transposase of class II (DNA transposase), for example, recognises a TA dinucleotide at each end of the transposable element, particularly within the repetitive sequences/terminal repeats of the transposable element. It also recognises a TA dinucleotide in the target sequence.

As mentioned above, the transposable element and the polynucleotide according to the second aspect may be part of the same vector. In this case, it is preferred that the polynucleotide is located external to the cloning site for inserting at least one polynucleotide of interest. It is particularly preferred that the polynucleotide according to the second aspect is located outside of the terminal repeats, e.g. inverted terminal repeats (ITRs) or long terminal repeats (LTR), flanking the cloning site for inserting the at least one polynucleotide of interest.

The transposable element provided with the kit/comprised in the kit may be derived from a prokaryotic or an eukaryotic transposable element, wherein the latter is preferred.

The transposable element may be a Class II or a DNA/DNA-based transposable element. The DNA/DNA-based transposable element comprises inverted terminal repeats (ITRs). It is recognized by a transposase of class II (DNA transposase). The transposable element may also be a Class I or a retrotransposable element. The retrotransposable element may be a long terminal repeat (LTR) retrotransposable element. The LTR retrotransposable element comprises long terminal repeats (LTRs). It is recognized by a transposase of class I (retrotransposase). Said transposase may also be designated as integrase.

In one preferred embodiment, the transposable element is a Class II or a DNA/DNA-based transposable element. In one more preferred embodiment, the transposable element is a PiggyBac transposable element, a sleeping beauty transposable element, a Hermes transposable element, a Helraiser transposable element, a TcBuster transposable element, a Teratorn transposable element or a Tol2 transposable element. Preferably, the PiggyBac transposable element is a wild-type PiggyBac transposable element, a hyperactive PiggyBac transposable element, a wild-type PiggyBac-like transposable element, or a hyperactive PiggyBac-like transposable element. The PiggyBac-like transposable element is more preferably selected from the group consisting of a PiggyBat transposable element, a PiggyBac-like transposable element from Xenopus tropicalis, and a PiggyBac-like transposable element from Bombyx mori.

The transposable element and/or the vector comprising the transposable element may further comprise elements that enhance expression (e.g. nuclear export signals, promoters, introns, terminators, enhancers, elements that affect chromatin structure, RNA export elements, IRES elements, CHYSEL elements, and/or Kozak sequences), selectable marker (e.g. DHFR, puromycine, hygromycin, zeocin, blasticidin, and/or neomycin), marker for in vivo monitoring (e.g. GFP or beta-galactosidase), a restriction endonuclease recognition site (e.g. a site for insertion of an exogenous nucleotide sequence such as a multiple cloning site), a recombinase recognition site (e.g. LoxP (recognized by Cre), FRT (recognized by Flp), or AttB/AttP (recognized by PhiC31)), insulators (e.g. MARs or UCOEs), viral replication sequences (e.g. SV40 ori).

The kit may comprise not only one but also more than one transposable element. The transposable elements may differ from each other, e.g. with respect to the cloning site and/or the specific composition of additional elements. This allows the cloning of diverse polynucleotides of interest into the different transposable elements.

In one embodiment, the kit is for the generation of a cell, in particular transgenic cell.

In one another embodiment, the kit further comprises instructions on how to generate the cell, in particular transgenic cell.

The kit may further comprise a container, wherein the single components of the kit are comprised. The kit may also comprise materials desirable from a commercial and user standpoint including a buffer(s), a reagent(s) and/or a diluent(s).

In an eight aspect, the present invention relates to a targeting system comprising (i) a transposable element comprising at least one polynucleotide of interest, and a polypeptide according to the first aspect, (ii) a transposable element comprising at least one polynucleotide of interest, and a polynucleotide according to the second aspect, (iii) a transposable element comprising at least one polynucleotide of interest, and a vector according to the third aspect, or (iv) a transposable element comprising at least one polynucleotide of interest, at least one heterologous GC Rich DNA Binding Domain (GRDBD), optionally associated with the transposable element, and a polypeptide comprising a transposase or a fragment or a derivative thereof having transposase function.

The targeting system may be comprised in/part of a cell or may be introduced into a cell. The introduction of the targeting system into a cell may take place via electroporation, transfection, injection, lipofection, or (viral) infection.

The cell may be an isolated cell (such as in cell culture or in cell line, e.g. stable cell line). The cell may also be a cell of a tissue outside of an organism. The cell may further be part of/comprised in an organism, e.g. eukaryotic multicellular organism. In this case, the insertion of the targeting system is effected in vivo.

In an alternative, a polypeptide comprising a transposase or a fragment, or a derivative thereof having transposase function is comprised in the targeting system without being connected to a GC Rich DNA Binding Domain (GRDBD), in particular GC Rich Binding Protein (GRBP) (see under (iv)). In this specific case, the polypeptide comprising a transposase or a fragment, or a derivative thereof having transposase function and the GRDBD, in particular GRBP, are comprised in the targeting system as distinct components. Preferably, the GRDBD, in particular GRBP, is associated with a binding molecule/moiety which is-after introduction into a cell-able to bind the transposase (e.g. via the N-terminus or C-terminus) forming a transposase, binding molecule/moiety and GRDBD, in particular GRBP, complex. This, of course, requires that the polypeptide comprising a transposase, or or a fragment, or a derivative thereof having transposase function comprises a binding domain allowing the binding molecule/moiety associated with the GRDBD, in particular GRBP, to bind. This binding domain is preferably a protein binding domain. Alternatively, the GRDBD, in particular GRBP, is associated with a binding molecule/moiety which is-after introduction into a cell-able to bind the transposable element. This, of course, requires that the transposable element comprises a binding domain allowing the binding molecule/moiety associated with the GRDBD, in particular GRBP, to bind. This binding domain is preferably a DNA binding domain.

The polypeptide comprising a transposase or a fragment or a derivative thereof having transposase function may be a recombinant, an artificial, and/or a heterologous polypeptide.

In one embodiment, the transposable element comprising at least one polynucleotide of interest is comprised in/part of a polynucleotide molecule, preferably a vector.

In one alternative embodiment, the transposable element comprising at least one polynucleotide of interest and the polynucleotide according to the second aspect are comprised in/part of a polynucleotide molecule, preferably a vector.

The transposable element may be a recombinant, an artificial, and/or a heterologous transposable element.

In one preferred embodiment, the transposable element is a Class II or a DNA/DNA-based transposable element. In one more preferred embodiment, the transposable element is a PiggyBac transposable element, a sleeping beauty transposable element, a Hermes transposable element, a Helraiser transposable element, a TcBuster transposable element, a Teratorn transposable element or a Tol2 transposable element. Preferably, the PiggyBac transposable element is a wild-type PiggyBac transposable element, a hyperactive PiggyBac transposable element, a wild-type PiggyBac-like transposable element, or a hyperactive PiggyBac-like transposable element. The PiggyBac-like transposable element is more preferably selected from the group consisting of a PiggyBat transposable element, a PiggyBac-like transposable element from Xenopus tropicalis, and a PiggyBac-like transposable element from Bombyx mori.

Preferably, the GC Rich DNA Binding Domain (GRDBD) is a GC Rich DNA Binding Protein (GRBP).

As to further preferred embodiments of the transposable element, it is referred to the fourth or seventh aspect of the present invention.

In a further aspect, the present invention relates to a targeting system comprising (i) a transposable element comprising at least one polynucleotide of interest and (ii) a polypeptide comprising a transposase or a fragment or a derivative thereof having transposase function, characterized in that the transposable element and/or the polypeptide comprising a transposase or a fragment or a derivative thereof having transposase function is directly associated (preferably via covalent fusion/attachment) or indirectly associated (preferably via a binding molecule) with a heterologous GC Rich DNA Binding Domain (GRDBD), preferably GC Rich Binding Protein (GRBP).

As to preferred embodiments of the transposable element, it is referred to the fourth and/or seventh aspect of the present invention.

In a further aspect, the present invention relates to a (transgenic) cell comprising a transposable element comprising at least one polynucleotide of interest, and a polypeptide according to the first aspect, a polynucleotide according to the second aspect, or a vector according to the third aspect.

As to further preferred embodiments with respect to the cell and the transposable element, it is referred to the fourth aspect of the present invention.

In a further aspect, the present invention relates to a (transgenic) cell comprising a heterologous transposable element which comprises at least one polynucleotide of interest, wherein the heterologous transposable element is predominantly, preferably exclusively, integrated/located in transcriptionally active genomic structures (euchromatin). More preferably, the heterologous transposable element is predominantly, preferably exclusively, integrated/located in (a) GC-rich DNA. Said cell had been treated with a targeting system according to the aspect.

As to further preferred embodiments with respect to the cell and the transposable element, it is referred to the fourth aspect of the present invention.

Various modifications and variations of the invention will be apparent to those skilled in the art without departing from the scope of invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art in the relevant fields are intended to be covered by the present invention.

### EXAMPLES

The disclosure is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications fall within the scope of the appended claims.

Also provided are transfected cell prepared by the methods disclosed herein.

In any of the constructs or vectors disclosed herein, additional transcriptional regulatory sequences and/or post-transcriptional regulatory sequences can be included. Transcriptional regulatory sequences can include, for example, promoters, enhancers and polyadenylation signals. Post-transcriptional regulatory sequences include, for example, introns and PREs.

In certain embodiments, a multiple cloning site (MCS), also known as a “polylinker,” is present in the vector to facilitate insertion of heterologous sequences. For example, a MCS can be disposed between a promoter and a polyadenylation signal, to facilitate insertion of transgene sequences. In vectors containing transgene sequences, the portion of the vector containing a promoter, transgene sequences a polyadenylation signal is denoted the “expression cassette.”

Promoters active in eukaryotic cells are known in the art. Exemplary eukaryotic promoters include, for example SV40 early promoter, SV40 late promoter, cytomegalovirus (CMV) promoter, cytomegalovirus major immediate early (CMV-MIE) promoter, EF1-alpha (translation elongation factor-1 α subunit) promoter, Ubc (ubiquitin C) promoter, PGK (phosphoglycerate kinase) promoter, actin promoter and others. See also Boshart et al., GenBank Accession No. K03104; Uetsuki et al. (1989) J. Biol. Chem. 264:5791-5798; Schorpp et al. (1996) Nucleic Acids Res. 24:1787-1788; Hamaguchi et al. (2000) J. Virology 74:10778-10784; Dreos et al. (2013) Nucleic Acids Res. 41 (D1): D157-D164 and the eukaryotic promoter database at http://epd.vital-it.ch, accessed on Jul. 16, 2014.

Cap independent translation initiation mechanisms are also known in the art. For example, IRES elements including but not limited to CVB3, HTLV, EMCV can recruit transcriptional machinery independent of canonical cap-dependent transcription activation such that a single mRNA sequence can code for 1 or more polypeptides.

Enhancers can also be included on the vector. Non-limiting examples include those in CMV promoter and intron A sequences. Five embryonic stem cell (ESC) transcription factors were previously shown to occupy super-enhancers (Oct4, Sox2, Nanog, Klf4, and Esrrb), and there are many additional transcription factors that contribute to the control of ESCs. Six additional transcription factors (Nr5a2, Prdm14, Tcfcp211, Smad3, Stat3, and Tcf3) occupy both typical enhancers and super-enhancers and that all of these are enriched in super-enhancers. Any of these or further known in the art can be used herein.

Polyadenylation signals that are active in eukaryotic cells are known in the art and include, but are not limited to, the SV40 polyadenylation signal, the bovine growth hormone (BGH) polyadenylation signal and the herpes simplex virus thymidine kinase gene polyadenylation signal. The polyadenylation signal directs 3′ end cleavage of pre-mRNA, polyadenylation of the pre-mRNA at the cleavage site and termination of transcription downstream of the polyadenylation signal. A core sequence AAUAAA is generally present in the polyadenylation signal. See also Cole et al. (1985) Mol. Cell. Biol. 5:2104-2113.

Exemplary introns that can be used in the vectors disclosed herein include the β-globin intron and the first intron of the human/mouse/rat/other species cytomegalovirus major immediate early (MIE) gene, also known as “intron A.”

Additional post-transcriptional regulatory elements that can be included in the vectors of the present disclosure include, without limitation, the 5′-untranslated region of CMV MIE, the human Hsp70 gene, the SP163 sequence from the vascular endothelial growth factor (VEGF) gene, and the tripartite leader sequence associated with adenovirus late mRNAs. See, for example, Mariati et al. (2010) Protein Expression and Purification 69:9-15.

In certain embodiments, the vectors disclosed herein contain nucleotide sequences encoding a selection marker that functions in eukaryotic cells (i.e., a eukaryotic selection marker), such that when appropriate selection is applied, cells that do not contain the selection marker die or grow appreciably more slowly that do cells that contain the selection marker. An exemplary selection marker that functions in eukaryotic cells is the glutamine synthetase (GS) gene; selection is applied by culturing cells in medium lacking glutamine or selection with L-Methioniene Sulfoximine or both. Another exemplary selection marker that functions in eukaryotic cells is the gene encoding resistance to neomycin (neo); selection is applied by culturing cells in medium containing neomycin, Geneticine or G418. Additional selection markers include dihydrofolate reductase (DHFR, imparts resistance to methotrexate), puromycin-N-acetyl transferase (provides resistance to puromycin) and hygromycin kinase (provides resistance to hygromycin B). Yet additional selection markers that function in eukaryotic cells are known in the art.

The sequences encoding the selection marker(s) described above are operatively linked to a promoter and a polyadenylation signal. As stated above, promoters and polyadenylation signals that function in eukaryotic cells are known in the art.

In certain embodiments, a vector as disclosed herein can contain two or more expression cassettes. For example, a vector containing two expression cassettes, one of which encodes an antibody heavy chain, and the other of which encodes an antibody light chain can be used for production of functional antibody molecules.

The vectors disclosed herein also contain a replication origin that functions in prokaryotic cells (i.e., a prokaryotic replication origin). Replication origins that functions in prokaryotic cells are known in the art and include, but are not limited to, the oriC origin of E. coli; plasmid origins such as, for example, the pSC101 origin, the pBR322 origin (rep) and the pUC origin; and viral (i.e., bacteriophage) replication origins. Methods for identifying procaryotic replication origins are provided, for example, in Sernova & Gelfand (2008) Brief. Bioinformatics 9 (5): 376-391.

The vectors disclosed herein also contain a selection marker that functions in prokaryotic cells (i.e., a prokaryotic selection marker). Selection markers that function in prokaryotic cells are known in the art and include, for example, sequences that encode polypeptides conferring resistance to any one of ampicillin, kanamycin, chloramphenicol, or tetracycline. An example of a polypeptide conferring resistance to ampicillin (and other beta-lactam antibiotics) is the beta-lactamase (bla) enzyme. Kanamycin resistance can result from activity of the neomycin phosphotransferase gene; and chloramphenicol resistance is mediated by chloramphenicol acetyl transferase.

Exemplary transgenes include any recombinant protein or e.g., hormones (such as, for example, growth hormone) erythropoietin, antibodies, polyclonal, monoclonal antibodies (e.g., rituximab), antibody conjugates, fusion proteins (e.g., IgG-fusion proteins), interleukins, CD proteins, MHC proteins, enzymes and clotting factors. Antibody heavy chains and antibody light chains can be expressed from separate vectors, or from the same vector containing two expression cassettes.

The present disclosure provides methods for expressing a recombinant polypeptide in a cell. The methods comprise introducing a vector as described herein into a cell and culturing the cell under conditions in which the vector is either transiently or stably maintained in the cell. Cells can be prokaryotic or eukaryotic, such as stable cell lines generated by targeted integration with CRISP/Cas9. Cultured eukaryotic cells, that can be used for expression of recombinant polypeptides, are known in the art. Such cells include fungal cells (e.g., yeast), insect cells, plant cells and mammalian cells. Accordingly, the present disclosure provides a cell comprising a vector as described herein.

Exemplary yeast cells include, but are not limited to, Trichoderma sp., Pichia pastoris, Schizosaccharomyces pombae and Saccharomyces cerevisiae. Exemplary insect cell lines include, but are not limited to, Sf9, Sf21, and Drosophila S2 cells. Exemplary plant cells include, but are not limited to, Arabidopsis cells and tobacco BY2 cells.

Cultured mammalian cell lines, useful for expression of recombinant polypeptides, include Chinese hamster ovary (CHO) cells, human embryonic kidney (HEK) cells, virally transformed HEK cells (e.g., HEK293 cells), NS0 cells, SP20 cells, CV-1 cells, baby hamster kidney (BHK) cells, 3T3 cells, Jurkat cells, HeLa cells, COS cells, PERC.6 cells, CAP® cells and CAP-T® cells (the latter two cell lines being commercially available from Cevec Pharmaceuticals, Cologne, Germany). A number of derivatives of CHO cells are also available such as, for example, CHO-DXB11, CHO-DG-44, CHO-K1, CHO-S, or engineered CHO cells such as CHO-M, CK1 SV CHO, and CHOZN. Mammalian primary cells can also be used.

In certain embodiments, the cells are cultured in a serum-free medium. For example, for manufacture of therapeutic proteins for administration to patients, expressing cells must be grown in serum-free medium. In additional embodiments, the cells have been pre-adapted for growth in serum-free medium prior to being used for polypeptide expression.

The vectors as described herein can be introduced into any of the aforementioned cells using methods that are known in the art. Such methods include, but are not limited to, polyethylene glycol (PEG)-mediated methods, electroporation, biolistic delivery (i.e., particle bombardment), protoplast fusion, DEAE-dextran-mediated methods, and calcium phosphate co-precipitation. See also, Sambrook et al. “Molecular Cloning: A Laboratory Manual,” Third Edition, Cold Spring Harbor Laboratory Press, 2001; and Ausubel et al., “Current Protocols in Molecular Biology,” John Wiley & Sons, New York, 1987 and periodic updates.

Standard methods for cell culture are known in the art. See, for example, R. I. Freshney “Culture of Animal Cells: A Manual of Basic Technique,” Fifth Edition, Wiley, New York, 2005.

### Example 1. GRDBD Linked Teratorn Transposases for GFP Integration

The teratorn transposase was used as an initial template to test the effect of retargeting transposases towards GC-Rich regions of the genome by linking said transposase with GC-Rich DNA binding proteins. Additionally, negative control constructs and alternative transcription factors were screened for their relative effect on gene integration efficiency and resulting transcription efficiency. In particular, the following GRDBD-transposase constructs were tested; Sp1-Teratorn (SEQ ID NO:77), Gpbp1-Teratorn (SEQ ID NO:78). The following non-GRDBD-transposase constructs were also tested; TBP-Teratorn (SEQ ID NO:80), MAU2-Teratorn (SEQ ID NO: 81), TFIIB-Teratorn (SEQ ID NO: 96), HMGBI-Teratorn (SEQ ID NO:97), CREB-Teratorn (SEQ ID NO:98), ARID2-Teratorn (SEQ ID NO:99). ARID2 encodes a member of the AT-rich interactive domain (ARID)-containing family of DNA-binding proteins. CREB is a transcription factor that regulates diverse cellular responses, including proliferation, survival, and differentiation. CREB is induced by a variety of growth factors and inflammatory signals and subsequently mediates the transcription of genes containing a cAMP-responsive element. CREB responsive promoters and elements are among the most highly in the genome. TBP and TFIIB bind to transcription initiation regions. HMGBI has many functions in the nucleus, mainly acting in NHEJ DNA repair pathways. MAU2 plays an important role in loading Cohesin onto DNA. It is expected that HMGBI/MAU2 would be relatively untargeted in their DNA binding site preference, TBP/TFIIB/CREB would have a stronger preference to bind near highly transcriptionally active locations in the genome, ARID2 would have a strong preference to bind near AT-rich regions of the genome, and GPBP1/Sp1 would have a strong preference to bind near GC-rich regions of the genome.

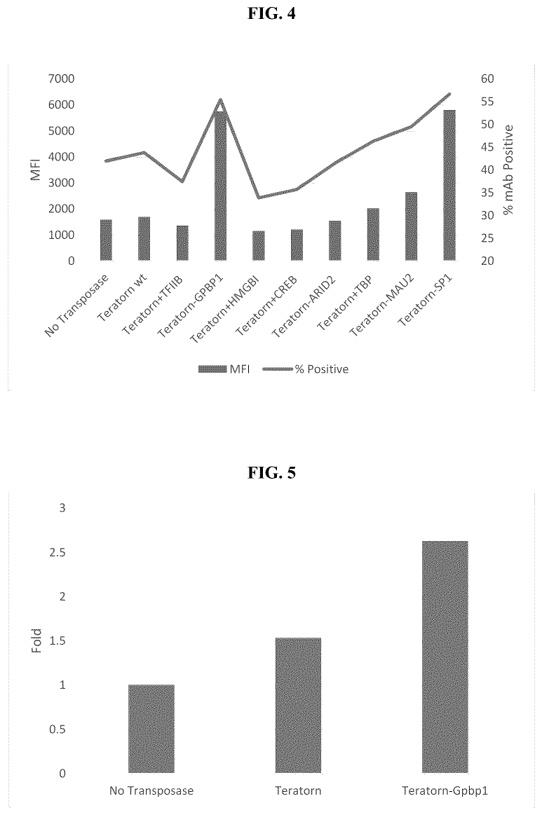

A GFP expressing plasmid was cloned, comprising at least the 5′ and 3′ inverted repeat elements of a Teratorn transposon (SEQ ID NO:86 and 87). The plasmid was scaled up and sequence confirmed. The circular transposase expression plasmids were co-transfected along with the circular plasmid containing the GFP gene using electroporation. The ratio of transposase to GFP plasmid was 1:1. The cells were allowed to recover in media supplemented with glutamine for three days at 37° C. and 5% CO2 until viability levels reached >97%. The cells were then assayed by flow cytometry for single cell fluorescence intensity and frequency of GFP positive cells. The results are shown in FIG. 4.

Strikingly, the difference between the expression levels and integration efficiency of GFP into the cells was barely above the background no transposase control for all but two conditions, the two GRDBD containing transposase variants. The addition of the GRDBD sequence domain onto the Teratorn transposase increased not only the fluorescence intensity of GFP positive cells, but also increased the number of GFP positive cells compared to the control Teratorn sample. Thus, the addition of the GRDBD to the teratorn transposase may not only improve expression by targeting the gene of interest to transcriptionally stable locations, but also may somehow improve integration efficiency of the transposon into the genome.

### Example 2. GRDBD Linked Teratorn Transposases for Antibody Expression

Celltheon's proprietary CHOK1 cells (SUPERCELL™) were transfected using electroporation, with circular plasmids encoding DNA expressing the Teratorn transposase (SEQ ID NO: 2), the DNA expressing the Gpbp1-Teratorn transposase (SEQ ID NO: 79), and no transposase, all in combination with a circular plasmid containing the gene of interest and first and second inverted repeat elements of the Teratorn transposon (SEQ ID NO:86, 87). A monoclonal antibody (mAb) was used as the gene of interest in the cotransfection and expression experiment.

Methionine sulfoximine (MSX) (EDM Millipore, Burlington, Mass.), was added 3 days post transfection, after recovery of the initial pool to >90% viability. Addition of methionine sulfoximine (MSX), an inhibitor of glutamine synthetase, is used to increase the expression of a gene of interest containing the Glutamine Synthetase (GS) selection marker in CHO cell lines.

Once the MSX-treated cells recovered they were subjected to a small scale fed batch production in 30 mL shake flasks to assess expression of the expressed protein from the stable cell lines. The cultures were seeded at 0.7×106 cells/mL in a basal production medium, and additional nutrients were fed on days 3-13. The cultures were harvested on day 14 and supernatants were analyzed for titer by Octet Protein A binding of the culture supernatant to the sensor surface. Resulting binding curves were compared to a standard curve to determine the concentration of antibody in the harvest samples.

Those pools transfected with a teratorn transposase showed an increase in expression as compared to those without any transposase as shown in FIG. 5. Further the pools developed using the GPBP1-Teratorn transposase showed an additional increase in expression as compared to the transposase pools with no Transposase or the wild type Teratorn Transposase.

The GPBP1-Teratorn transposase imparts a semi-targeted mechanism of transfection that targets open, GC-rich chromatin which are areas of active transcription in the genome. When the transposon is integrated in combination with the transposase, the full vector sequence between the first and second inverted repeat elements of the transposon is integrated. Transfection of the transposon without the transposase results in random integration into the chromosome, and as a result, the full vector sequence between the first and second inverted repeat elements of the transposon may not be integrated into an active site. Transfection of the transposon with the wildtype transposase improves gen integration efficiency in comparison to the no transposase transfection, however, does not efficiently target transcriptionally active regions of the genome.

### Example 3. GRDBD Linked PiggyBac Transposases for Antibody Expression

Celltheon's proprietary CHOK1 cells (SUPERCELL™) were transfected using electroporation, with circular plasmids encoding DNA expressing the Hyperactive PiggyBac transposase, PB_CTVar9 (SEQ ID NO: 13), MAU2-PB_CTVar9 (SEQ ID NO:76), CREB-PB_CTVar9 (SEQ ID NO: 100), TBP-PB_CTVar9 (SEQ ID NO:75), GPBP1-PB_CTVar9 (SEQ ID NO: 74), Sp1-PB_CTVar9 (SEQ ID NO:72), Sp1_ZFN-PB_CTVar9 (SEQ ID NO:73), and no transposase, all in combination with a circular plasmid containing the gene of interest and first and second inverted repeat elements of the PiggyBac transposon (SEQ ID NO:84, 85). A monoclonal antibody (mAb) was used as the gene of interest in the cotransfection and expression experiment.

Methionine sulfoximine (MSX) (EDM Millipore, Burlington, Mass.), was added 3 days post transfection, after recovery of the initial pool to >90% viability. Addition of methionine sulfoximine (MSX), an inhibitor of glutamine synthetase, is used to increase the expression of a gene of interest containing the Glutamine Synthetase (GS) selection marker in CHO cell lines.

Once the MSX-treated cells recovered they were subjected to a small scale fed batch production in 30 mL shake flasks to assess expression of the expressed protein from the stable cell lines. The cultures were seeded at 0.7×106 cells/mL in a basal production medium, and additional nutrients were fed on days 3-13. The cultures were harvested on day 14 and supernatants were analyzed for titer by Octet Protein A binding of the culture supernatant to the sensor surface. Resulting binding curves were compared to a standard curve to determine the concentration of antibody in the harvest samples.

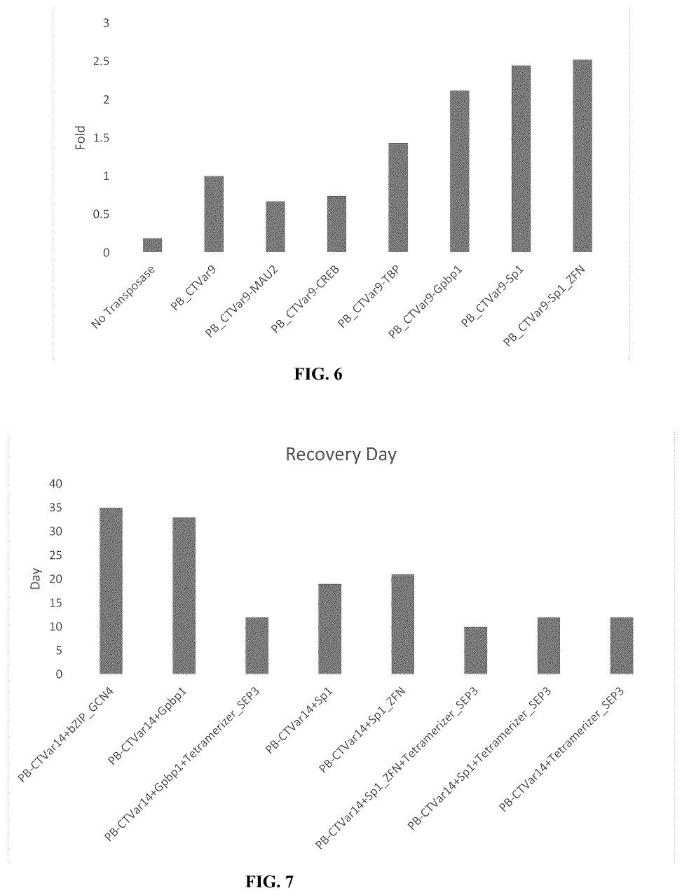

Those pools transfected with a hyperactive PiggyBac transposase showed an increase in expression as compared to those without any transposase. Further the pools developed using the GPBP1-PB_CTVar9, Sp1-PB_CTVar9, Sp1_ZFN-PB_CTVar9, and TBP-PB_CTVar9, transposase showed an additional increase in expression as compared to the PB_CTVar9 pool as shown in FIG. 6. Sp1 and Gpbp1 are transcription factors with GC-rich binding domains, Sp1_ZFN is the Zinc Finger DNA binding domain of Sp1, TBP binds to transcription initiation sites, CREB binds to highly transcribed genes, and MAU2 is a nonspecific DNA binding sequence. The MAU2-PB_CTVar9 and CREB-PB_CTVar9 samples showed similar or lower titers to the PB_CTVar9 control. Strikingly, the three highest titers in the set of transposases assessed all had GC Rich DNA binding domains. In particular, Sp1_ZFN-PB_CTVar9 yielded the highest titers, indicating that the core functional element which increases titers in comparison to the unmodified PB_CTVar9 identified in the Sp1 transcription factor, is in fact the GC-rich binding sequence of the transcription factor.

The findings in FIG. 6 support the idea that these novel, modified transposases impart a semi-targeted mechanism of integration that targets open, GC-rich chromatin which are areas of active transcription in the genome.

### Example 4. Tetramerization Linked PiggyBac-Like Transposases for Antibody Expression

Celltheon's proprietary CHOK1 cells (SUPERCELL™) were transfected using electroporation, with circular plasmids encoding DNA expressing the Hyperactive PiggyBac-like transposase, PB_CTVar14 (SEQ ID NO: 105), PB-CTVar14+bZIP_GCN4 (SEQ ID NO: 114) Gpbp1-PB_CTVar14 (SEQ ID NO:115), PB-CTVar14+Gpbp1+Tetramerizer_SEP3 (SEQ ID NO: 116), PB-CTVar14+Sp1 (SEQ ID NO:117), PB-CTVar14+Sp1_ZFN (SEQ ID NO:118), PB-CTVar14+Sp1_ZFN+Tetramerizer_SEP3 (SEQ ID NO:119), PB-CTVar14+Sp1+Tetramerizer_SEP3 (SEQ ID NO:120), and PB-CTVar14+Tetramerizer (SEQ ID NO: 121), all in combination with a circular plasmid containing the gene of interest and first and second inverted repeat elements of the PiggyBac transposon (SEQ ID NO:124, 125). A monoclonal antibody (mAb) was used as the gene of interest in the cotransfection and expression experiment.

Methionine sulfoximine (MSX) (EDM Millipore, Burlington, Mass.), was added 3 days post transfection, after recovery of the initial pool to >90% viability. Addition of methionine sulfoximine (MSX), an inhibitor of glutamine synthetase, is used to increase the expression of a gene of interest containing the Glutamine Synthetase (GS) selection marker in CHO cell lines.

Once the MSX-treated cells recovered they were subjected to a small scale fed batch production in 30 mL shake flasks to assess expression of the expressed protein from the stable cell lines. The cultures were seeded at 0.7×106 cells/mL in a basal production medium, and additional nutrients were fed on days 3-13. The cultures were harvested on day 14 and supernatants were analyzed for titer by Octet Protein A binding of the culture supernatant to the sensor surface. Resulting binding curves were compared to a standard curve to determine the concentration of antibody in the harvest samples.

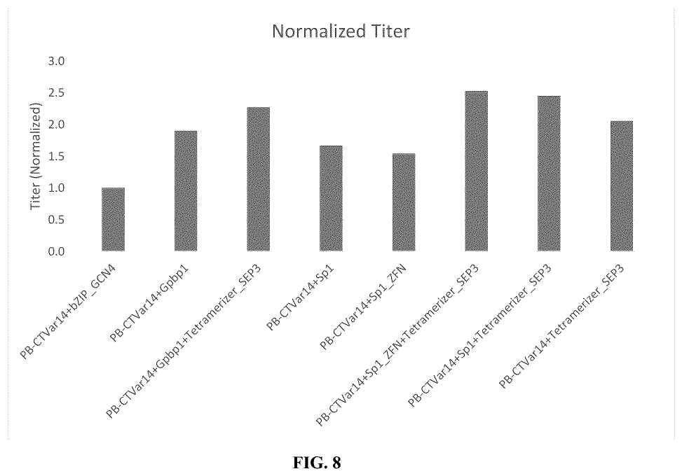

Those pools transfected with a tetramerization domain containing transposase sequence showed an improvement in time to recover pools from selective pressure, implying a more rapid and consistent integration efficiency as compared to those without a tetramerization domain as shown in FIG. 7. Further the pools developed using the PB-CTVar14+Gpbp1+Tetramerizer_SEP3 and PB-CTVar14+Sp1_ZFN+Tetramerizer_SEP3 transposase showed an additional increase in expression as compared to the PB_CTVar14 pool as shown in FIG. 8. bZIP_GCN4 is a dimerization domain whereas SEP3 is a tetramerization domain. The long and slow recovery time of the bZIP_GCN4 containing sequences shown in FIG. 7 imply no improvement in transposition frequency and potentially a detriment to transposition frequency. The rapid recovery time of the SEP3 containing sequences shown in FIG. 7 imply an improved transposition frequency and reduce timelines in the development of stable expression cell lines. Strikingly, the highest titers in the set of transposases assessed all had tetramerization domains. In particular, PB-CTVar14+Sp1_ZFN+Tetramerizer_SEP3 yielded the highest titers, indicating that the tetramerization domain increases titers in comparison to the unmodified PB_CTVar14+Sp1.

The findings in FIG. 7 support the idea that these novel, modified transposases impart an improved transposition frequency of integration.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

What is claimed is:

1. A recombinant polypeptide comprising a transposase or fragment or a derivative thereof having transposase function and at least one heterologous GC Rich DNA Binding Domain (GRDBD) or at least one heterologous multimerization domain.

2. The polypeptide of claim 1, wherein the at least one heterologous GRDBD is a GC Rich DNA Binding Protein (GRBP).

3. The polypeptide of claim 2, wherein the at least one heterologous GRBP is a naturally occurring transcription factor or fragment thereof which preferentially binds to GC-Rich transcription factor sites.

4. The polypeptide of claim 3, wherein the naturally occurring GRDBD recognizing GC-rich DNA is a transcription factor or DNA binding region of a transcription factor with at least 70% sequence identity to SEQ ID NO: 14-39, 66-71 or SEQ ID NO: 40-65.

5. The polypeptide of claim 4, wherein the transcription factor is transcription factor with at least 70% sequence identity to Gpbp1 (SEQ ID NO: 66), Sp1 (SEQ ID NO:14), or Sp1 ZEN (SEQ ID NO:40).

6. The polypeptide of claim 1, wherein the at least one heterologous multimerization domain is a tetramerization domain.

7. The polypeptide of claim 6, wherein the tetramerization domain is a naturally occurring MADS transcription factor or fragment thereof which imparts constitutive tetramerization.

8. The polypeptide of claim 7, wherein the naturally occurring MADS transcription factor has at least 70% sequence identity to SEQ ID NO: 122.

9. The polypeptide of claim 1, wherein the transposase is selected from the group consisting of a wild-type PiggyBac transposase, a hyperactive PiggyBac transposase, a sleeping beauty transposase, a Teratorn transposase, a Hermes transposase, a TcBuster transposase, a Helraiser transposase, and a Tol2 transposase.

10. A hyperactive transposase polypeptide sequence having at least 90% sequence identity to SEQ ID NO:101, and comprising at least one of the following amino acid substitutions in SEQ ID NO:101: a tryptophan for histidine at position 149; a phenylalanine for a tryptophan at position 167; an arginine for a lysine at position 168; a methionine for a glutamine at position 281; a glycine for a glutamine at position 315; a serine for a lysine at position 372; a glutamine for a lysine at position 440; an asparagine for an arginine at position 462; a valine for an asparagine at position 488; a serine for a proline at position 489; a methionine for a leucine at position 502; an arginine for a lysine at position 503; an asparagine for a glutamate at position 504; a threonine for an alanine at position 542; deletion of positions 544 to 548.

11. The polypeptide sequence of claim 10 comprising at least 90% sequence identity to SEQ ID NO: 2, and comprising:
an amino acid substitution at position 440 and 504,
wherein the substitution at position 440 is a substitution of a Glutamine for Lysine (Q440K), and
wherein the substitution at position 504 is a substitution of an Asparagine for a Glutamate (N504E).


SEQUENCE LISTING (100 sequences)

SEQ ID NO: 1 - protein, 594 aa, Trichoplusia ni
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 2 - protein, 830 aa, Oryzias latipes
MNKGRKRTLRSRSRPLAHKRGTRAVEPEDPRPDSQGQRGGPKPGRYEPYDWDSDSDGGHRDGGGHGRVGRKTRAGRRDKAREPDAAAVEPVGPRHDSEGQRGAPKPGRYEPYDWDSDSDGGDGGGHGPPRDEALEPDAAAVEPEGPRHDSEGQGGGDGDRGGHGQVGRETLEDRRSDPPRDEALEPDAAAVEPEGPRHDSEGQGGGDGDRGGHGQVGRETLEDRRSDPPRDEAHEPDDGAVEPGGPQHELEGQGRGDEPSDWDLYECETDVDELDGSQSETEEQHRGPRSSGEEDVEEAVEEDAEEDIEEEPAAESASVYPDTFSSKNGEIKWSANERDRSSVRASPPHQASGPTTEAREGAQDILSSFLLFFTPNVERLILIATNRHSADKAHALKTPKPMDEIELRAYVGLLILAGVYRSRGEALVSLWEPECGRPIFGATMSLRSFYQRSATLRFDDRSTRASRSASDRLAAVREVWEEWCDRLPRLYDIGPEVTVDERMVPFKRCSFRQYIPSKPTKYGLKLWVACDARTSYAWRVQPYMGKLASGARETNLASRVVSDLTRGLEHRNVTCDNFFTSYALATELLKRNITLLGTIKSNKPELPPELASARRREALSSRFAFTDCAAALSYVPKKNKNVLLLSTKHSRVEICHERRDKKPEMILDYNKTKGGVDNLDKLLATYSCRRMTKRWPLAMFHNMIDMSAYNAYVIWREMHPDWMPWNRNRRRIFLEQLGKDLVRPLVLRRKRVPRARPAYDLMVSMQEAGGTSARSEGKRKRCQICPRSKDTKTLISCKSCGKSICRGCTVPFCPLCSERQGRDGDTDASQ

SEQ ID NO: 3 - protein, 612 aa, Musca domestica
MEKMDNLEVKAKINQGLYKITPRHKGTSFIWNVLADIQKEDDTLVEGWVFCRKCEKVLKYTTRQTSNLCRHKCCASLKQSRELKTVSADCKKEAIEKCAQWVVRDCRPFSAVSGSGFIDMIKFFIKVGAEYGEHVNVEELLPSPITLSRKVTSDAKEKKALISREIKSAVEKDGASATIDLWTDNYIKRNFLGVTLHYHENNELRDLILGLKSLDFERSTAENIYKKLKAIFSQFNVEDLSSIKFVTDRGANVVKSLANNIRINCSSHLLSNVLENSFEETPELNMPILACKNIVKYFKKANLQHRLRSSLKSECPTRWNSTYTMLRSILDNWESVIQILSEAGETQRIVHINKSIIQTMVNILDGFERIFKELQTCSSPSLCFVVPSILKVKEICSPDVGDVADIAKLKVNIIKNVRIIWEENLSIWHYTAFFFYPPALHMQQEKVAQIKEFCLSKMEDLELINRMSSFNELSATQLNQSDSNSHNSIDLTSHSKDISTTSFFFPQLTQNNSREPPVCPSDEFEFYRKEIVILSEDFKVMEWWNLNSKKYPKLSKLALSLLSIPASSAASERTFSLAGNIITEKRNRIGQQTVDSLLFLNSFYKNFCKLDI

SEQ ID NO: 4 - protein, 636 aa, Tribolium castaneum
MMLNWLKSGKLESQSQEQSSCYLENSNCLPPTLDSTDIIGEENKAGTTSRKKRKYDEDYLNFGFTWTGDKDEPNGLCVICEQVVNNSSLNPAKLKRHLDTKHPTLKGKSEYFKRKCNELNQKKHTFERYVRDDNKNLLKASYLVSLRIAKQGEAYTIAEKLIKPCTKDLTTCVFGEKFASKVDLVPLSDTTISRRIEDMSYFCEAVLVNRLKNAKCGFTLQMDESTDVAGLAILLVFVRYIHESSFEEDMLFCKALPTQTTGEEIFNLLNAYFEKHSIPWNLCYHICTDGAKAMVGVIKGVIARIKKLVPDIKASHCCLHRHALAVKRIPNALHEVLNDAVKMINFIKSRPLNARVFALLCDDLGSLHKNLLLHTEVRWLSRGKVLTRFWELRDEIRIFFNEREFAGKLNDTSWLQNLAYIADIFSYLNEVNLSLQGPNSTIFKVNSRINSIKSKLKLWEECITKNNTECFANLNDFLETSNTALDPNLKSNILEHLNGLKNTFLEYFPPTCNNISWVENPFNECGNVDTLPIKEREQLIDIRTDTTLKSSFVPDGIGPFWIKLMDEFPEISKRAVKELMPFVTTYLCEKSFSVYVATKTKYRNRLDAEDDMRLQLTTIHPDIDNLCNNKQAQKSH

SEQ ID NO: 5 - protein, 597 aa, synthetic construct
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVTRTSALNIVRSQRGPTRMCRNIYDPLLCFKLFFTDEIISEIVKHTNAEISLKRRESMTGATWKDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRQYMPNKPSKYGIKILMMCDSGTKYMINGMPYLGRQTQTNGVPLGEYYVKELSKPVHGSCRNITCDNWFTSIPLAKNLLQEPYKLTIVGTVRKNKREIPEVLKNSRSRPVGTSMFCFDGPLTLVSYKPKPAKMVYLLSSCHHDASVNESTGKPQMVMYYNKTKGGVDTLDQMCSVMTCSRKTRRWPMALLYGMINIACINSFIIYSHNVSSKGEKVQSRKKFLKELYMSLTSSFMRKRLEAPTLKRYLRDNISNILPNEVPGTSDDSTEEPVMKKRTYCHYCPSKIRRKANASCKKCKKVICREHNIDICQSCF

SEQ ID NO: 6 - protein, 594 aa, synthetic construct
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKHTNAEISLKRRESMTGATWKDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRQTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNKTKGGVDTLDQMCSVMTCSRKTNRWPMALLYGMINIACINSFIIYSHNVSSKGEKVQSRKKFLKELYMSLTSSFMRKRLEAPTLKRYLRDNISNILPNEVPGTSDDSTEEPVMKKRTYCHYCPSKIRRKANASCKKCKKVICREHNIDICQSCF

SEQ ID NO: 7 - protein, 594 aa, synthetic construct
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKHTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRQTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFLKELYMSLTSSFMRKRLEAPTLKRYLRDNISNILPNEVPGTSDDSTEEPVMKKRTYCHYCPSKIRRKANASCKKCKKVICREHNIDMCQSCF

SEQ ID NO: 8 - protein, 580 aa, synthetic construct
MQNDDELVGEDPDEEIQDHVDEDDVQEDDEEAFIDEVHEVQPDDDGSESEKSDFVIEQPGSSLASNRILTLPQRTIRGKNKHCWSTSKSTRRGRVRALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASIDESTGKPQMVMYYNKTKGGVDTLDQMCSVMTCSRKTNRWPMALLYGMINIACINSFIIYSHNNPSKGEKVQSRKKFMRNLYMSLTSSFMRKRLEAPTLKRYLRDNISNILPNEVPGPAEEPEPEPVMKKRTYCTYCPSKIRRKANASCKKCKKVICREHNIDMCQSCF

SEQ ID NO: 9 - protein, 580 aa, synthetic construct
MQNDDELVGEDPDSEISDHVSEDDVQSDTEEAFIDEVHEVQPDDDGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASIDESTGKPQMVMYYNKTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGPAEEPEPEPVMKKRTYCTYCPSKIRRKANASCKKCKKVICREHNIDMCQSCF

SEQ ID NO: 10 - protein, 580 aa, synthetic construct
MQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGPAEEPEPEPVMKKRTYCTYCPSKIRRKANASCKKCKKVICREHNIDMCQSCF

SEQ ID NO: 11 - protein, 580 aa, synthetic construct
MQNDDELVGEDPDEEIQDHVSEDDVQEDDEEAFIDEVHEVQPDDDGSESEKSDFVIEQPGSSLASNRILTLPQRTIRGKNKHCWSTSKSTRRGRVRALNIVRSQRGPTRMCRNIYDPLLCFKLFFTDEIISEIVKHTNAEISLKRRESMTGATWKDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRQYMPNKPSKYGIKILMMCDSGTKYMINGMPYLGRQTQTNGVPLGEYYVKELSKPVHGSCRNITCDNWFTSIPLAKNLLQEPYKLTIVGTVRKNKREIPEVLKNSRSRPVGTSMFCFDGPLTLVSYKPKPAKMVYLLSSCHHDASVNESTGKPQMVMYYNQTKGGVDTLDQMCSVMTCSRKTRRWPMALLYGMINIACINSFIIYSHNNPSKGEKVQSRKKFLKELYMSLTSSFMRKRLEAPTLKRYLRDNISNILPNEVPGPAEEPEPEPVMKKRTYCHYCPSKIRRKANASCKKCKKVICREHNIDMCQSCF

SEQ ID NO: 12 - protein, 580 aa, synthetic construct
MQNDDELVGEDPDSEISDHVSEDDVQSDTEEAFIDEVHEVQPDDDGSEILDEQNVIEQPGSSLASNRILTLPQRTIRGKNKHCWSTSKSTRRSRVSALNIVRSQRGPTRMCRNIYDPLLCFKLFFTDEIISEIVKHTNAEISLKRRESMTGATWKDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRQTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFLKELYMSLTSSFMRKRLEAPTLKRYLRDNISNILPNEVPGPAEEPEPEPVMKKRTYCHYCPSKIRRKANASCKKCKKVICREHNIDMCQSCF

SEQ ID NO: 13 - protein, 584 aa, synthetic construct
MGSSYPFALASEPDEEIQETLDEDDDSEEDDVEDVSNDLEEELTDSDDDGSESEKSDFVIEQPGSSLASNRILTLPQRTIRGKNKHCWSTSKSTRRSRVSALNIVRSQRGPTRMCRNIYDPLLCFKLFFTDEIISEIVKHTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRQTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFLKELYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 14 - protein, 785 aa, Homo sapiens
MSDQDHSMDEMTAVVKIEKGVGGNNGGNGNGGGAFSQARSSSTGSSSSTGGGGQESQPSPLALLAATCSRIESPNENSNNSQGPSQSGGTGELDLTATQLSQGANGWQIISSSSGATPTSKEQSGSSTNGSNGSESSKNRTVSGGQYVVAAAPNLQNQQVLTGLPGVMPNIQYQVIPQFQTVDGQQLQFAATGAQVQQDGSGQIQIIPGANQQIITNRGSGGNIIAAMPNLLQQAVPLQGLANNVLSGQTQYVTNVPVALNGNITLLPVNSVSAATLTPSSQAVTISSSGSQESGSQPVTSGTTISSASLVSSQASSSSFFTNANSYSTTTTTSNMGIMNFTTSGSSGTNSQGQTPQRVSGLQGSDALNIQQNQTSGGSLQAGQQKEGEQNQQTQQQQILIQPQLVQGGQALQALQAAPLSGQTFTTQAISQETLQNLQLQAVPNSGPIIIRTPTVGPNGQVSWQTLQLQNLQVQNPQAQTITLAPMQGVSLGQTSSSNTTLTPIASAASIPAGTVTVNAAQLSSMPGLQTINLSALGTSGIQVHPIQGLPLAIANAPGDHGAQLGLHGAGGDGIHDDTAGGEEGENSPDAQPQAGRRTRREACTCPYCKDSEGRGSGDPGKKKQHICHIQGCGKVYGKTSHLRAHLRWHTGERPFMCTWSYCGKRFTRSDELQRHKRTHTGEKKFACPECPKRFMRSDHLSKHIKTHQNKKGGPGVALSVGTLPLDSGAGSEGSGTATPSALITTNMVAMEAICPEGIARLANSGINVMQVADLQSINISGNGF

SEQ ID NO: 15 - protein, 667 aa, Homo sapiens
MSDPQTSMAATAAVSPSDYLQPAASTTQDSQPSPLALLAATCSKIGPPAVEAAVTPPAPPQPTPRKLVPIKPAPLPLSPGKNSFGILSSKGNILQIQGSQLSASYPGGQLVFAIQNPTMINKGTRSNANIQYQAVPQIQASNSQTIQVQPNLTNQIQIIPGTNQAIITPSPSSHKPVPIKPAPIQKSSTTTTPVQSGANVVKLTGGGGNVTLTLPVNNLVNASDTGAPTQLLTESPPTPLSKTNKKARKKSLPASQPPVAVAEQVETVLIETTADNIIQAGNNLLIVQSPGGGQPAVVQQVQVVPPKAEQQQVVQIPQQALRVVQAASATLPTVPQKPSQNFQIQAAEPTPTQVYIRTPSGEVQTVLVQDSPPATAAATSNTTCSSPASRAPHLSGTSKKHSAAILRKERPLPKIAPAGSIISLNAAQLAAAAQAMQTININGVQVQGVPVTITNTGGQQQLTVQNVSGNNLTISGLSPTQIQLQMEQALAGETQPGEKRRRMACTCPNCKDGEKRSGEQGKKKHVCHIPDCGKTFRKTSLLRAHVRLHTGERPFVCNWFFCGKRFTRSDELQRHARTHTGDKRFECAQCQKRFMRSDHLTKHYKTHLVTKNFCLRQNLQLGSSASFVFSLGELGEGTPEEGAVVSQSDPHWDQELIGTQGLRCCAD

SEQ ID NO: 16 - protein, 781 aa, Homo sapiens
MTAPEKPVKQEEMAALDVDSGGGGGGGGGHGEYLQQQQQHGNGAVAAAAAAQDTQPSPLALLAATCSKIGPPSPGDDEEEAAAAAGAPAAAGATGDLASAQLGGAPNRWEVLSATPTTIKDEAGNLVQIPSAATSSGQYVLPLQNLQNQQIFSVAPGSDSSNGTVSSVQYQVIPQIQSADGQQVQIGFTGSSDNGGINQESSQIQIIPGSNQTLLASGTPSANIQNLIPQTGQVQVQGVAIGGSSFPGQTQVVANVPLGLPGNITFVPINSVDLDSLGLSGSSQTMTAGINADGHLINTGQAMDSSDNSERTGERVSPDINETNTDTDLFVPTSSSSQLPVTIDSTGILQQNTNSLTTSSGQVHSSDLQGNYIQSPVSEETQAQNIQVSTAQPVVQHLQLQESQQPTSQAQIVQGITPQTIHGVQASGQNISQQALQNLQLQLNPGTFLIQAQTVTPSGQVTWQTFQVQGVQNLQNLQIQNTAAQQITLTPVQTLTLGQVAAGGAFTSTPVSLSTGQLPNLQTVTVNSIDSAGIQLHPGENADSPADIRIKEEEPDPEEWQLSGDSTLNTNDLTHLRVQVVDEEGDQQHQEGKRLRRVACTCPNCKEGGGRGTNLGKKKQHICHIPGCGKVYGKTSHLRAHLRWHSGERPFVCNWMYCGKRFTRSDELQRHRRTHTGEKKFVCPECSKRFMRSDHLAKHIKTHQNKKGIHSSSTVLASVEAARDDTLITAGGTTLILANIQQGSVSGIGTVNTSATSNQDILTNTEIPLQLVTVSGNETME

SEQ ID NO: 17 - protein, 784 aa, Homo sapiens
MSDQKKEEEEEAAAAAAMATEGGKTSEPENNNKKPKTSGSQDSQPSPLALLAATCSKIGTPGENQATGQQQIIIDPSQGLVQLQNQPQQLELVTTQLAGNAWQLVASTPPASKENNVSQPASSSSSSSSSNNGSASPTKTKSGNSSTPGQFQVIQVQNPSGSVQYQVIPQLQTVEGQQIQINPTSSSSLQDLQGQIQLISAGNNQAILTAANRTASGNILAQNLANQTVPVQIRPGVSIPLQLQTLPGTQAQVVTTLPINIGGVTLALPVINNVAAGGGTGQVGQPAATADSGTSNGNQLVSTPTNTTTSASTMPESPSSSTTCTTTASTSLTSSDTLVSSADTGQYASTSASSSERTIEESQTPAATESEAQSSSQLQPNGMQNAQDQSNSLQQVQIVGQPILQQIQIQQPQQQIIQAIPPQSFQLQSGQTIQTIQQQPLQNVQLQAVNPTQVLIRAPTLTPSGQISWQTVQVQNIQSLSNLQVQNAGLSQQLTITPVSSSGGTTLAQIAPVAVAGAPITLNTAQLASVPNLQTVSVANLGAAGVQVQGVPVTITSVAGQQQGQDGVKVQQATIAPVTVAVGGIANATIGAVSPDQLTQVHLQQGQQTSDQEVQPGKRLRRVACSCPNCREGEGRGSNEPGKKKQHICHIEGCGKVYGKTSHLRAHLRWHTGERPFICNWMFCGKRFTRSDELQRHRRTHTGEKRFECPECSKRFMRSDHLSKHVKTHQNKKGGGTALAIVTSGELDSSVTEVLGSPRIVTVAAISQDSNPATPNVSTNMEEF

SEQ ID NO: 18 - protein, 442 aa, Homo sapiens
MGGRAAGRRGCGCFPTLTDHGAGRGPSDASPSHFAPASSKAGWRLSAPSSLARSARGPHVQDRTPSASPDLGKHSPLALLAATCSRIGQPGAAAPPDFLQVPYDPALGSPSRLFHPWTADMPAHSPGALPPPHPSLGLTPQKTHLQPSFGAAHELPLTPPADPSYPYEFSPVKMLPSSMAALPASCAPAYVPYAAQAALPPGYSNLLPPPPPPPPPPTCRQLSPNPAPDDLPWWSIPQAGAGPGASGVPGSGLSGACAGAPHAPRFPASAAAAAAAAAALQRGLVLGPSDFAQYQSQIAALLQTKAPLAATARRCRRCRCPNCQAAGGAPEAEPGKKKQHVCHVPGCGKVYGKTSHLKAHLRWHTGERPFVCNWLFCGKSFTRSDELQRHLRTHTGEKRFACPECGKRFMRSDHLAKHVKTHQNKKLKVAEAGVKREDARDL

SEQ ID NO: 19 - protein, 376 aa, Homo sapiens
MLTAVCGSLGSQHTEAPHASPPRLDLQPLQTYQGHTSPEAGDYPSPLQPGELQSLPLGPEVDFSQGYELPGASSRVTCEDLESDSPLAPGPFSKLLQPDMSHHYESWFRPTHPGAEDGSWWDLHPGTSWMDLPHTQGALTSPGHPGALQAGLGGYVGDHQLCAPPPHPHAHHLLPAAGGQHLLGPPDGAKALEVAAPESQGLDSSLDGAARPKGSRRSVPRSSGQTVCRCPNCLEAERLGAPCGPDGGKKKHLHNCHIPGCGKAYAKTSHLKAHLRWHSGDRPFVCNWLFCGKRFTRSDELQRHLQTHTGTKKFPCAVCSRVFMRSDHLAKHMKTHEGAKEEAAGAASGEGKAGGAVEPPGGKGKREAEGSVAPSN

SEQ ID NO: 20 - protein, 431 aa, Homo sapiens
MASSLLEEEVHYGSSPLAMLTAACSKFGGSSPLRDSTTLGKAGTKKPYSVGSDLSASKTMGDAYPAPFTSTNGLLSPAGSPPAPTSGYANDYPPFSHSFPGPTGTQDPGLLVPKGHSSSDCLPSVYTSLDMTHPYGSWYKAGIHAGISPGPGNTPTPWWDMHPGGNWLGGGQGQGDGLQGTLPTGPAQPPLNPQLPTYPSDFAPLNPAPYPAPHLLQPGPQHVLPQDVYKPKAVGNSGQLEGSGGAKPPRGASTGGSGGYGGSGAGRSSCDCPNCQELERLGAAAAGLRKKPIHSCHIPGCGKVYGKASHLKAHLRWHTGERPFVCNWLFCGKRFTRSDELERHVRTHTREKKFTCLLCSKRFTRSDHLSKHQRTHGEPGPGPPPSGPKELGEGRSTGEEEASQTPRPSASPATPEKAPGGSPEQSNLLEI

SEQ ID NO: 21 - protein, 508 aa, Homo sapiens
MATSLLGEEPRLGSTPLAMLAATCNKIGSPSPSPSSLSDSSSSFGKGFHPWKRSSSSSSASCNVVGSSLSSFGVSGASRNGGSSSAAAAAAAAAAAAAALVSDSFSCGGSPGSSAFSLTSSSAAAAAAAAAAAASSSPFANDYSVFQAPGVSGGSGGGGGGGGGGSSAHSQDGSHQPVFISKVHTSVDGLQGIYPRVGMAHPYESWFKPSHPGLGAAGEVGSAGASSWWDVGAGWIDVQNPNSAAALPGSLHPAAGGLQTSLHSPLGGYNSDYSGLSHSAFSSGASSHLLSPAGQHLMDGFKPVLPGSYPDSAPSPLAGAGGSMLSAGPSAPLGGSPRSSARRYSGRATCDCPNCQEAERLGPAGASLRRKGLHSCHIPGCGKVYGKTSHLKAHLRWHTGERPFVCNWLFCGKRFTRSDELQRHLRTHTGEKRFACPVCNKRFMRSDHLSKHVKTHSGGGGGGGSAGSGSGGKKGSDTDSEHSAAGSPPCHSPELLQPPEPGHRNGLE

SEQ ID NO: 22 - protein, 484 aa, Homo sapiens
MATSILGEEPRFGTTPLAMLAATCNKIGNTSPLTTLPESSAFAKGGFHPWKRSSSSCNLGSSLSGFAVATGGRGSGGLAGGSGAANSAFCLASTSPTSSAFSSDYGGLFSNSAAAAAAAAGVSPQEAGGQSAFISKVHTTAADGLYPRVGMAHPYESWYKSGFHSTLAAGEVTNGAASSWWDVHSSPGSWLEVQNPAGGLQSSLHSGAPQASLHSQLGTYNPDFSSLTHSAFSSTGLGSSAAAASHLLSTSQHLLAQDGFKPVLPSYSDSSAAVAAAAASAMISGAAAAAAGGSSARSARRYSGRATCDCPNCQEAERLGPAGASLRRKGLHSCHIPGCGKVYGKTSHLKAHLRWHTGERPFVCNWLFCGKRFTRSDELQRHLRTHTGEKRFACPVCNKRFMRSDHLSKHIKTHNGGGGGKKGSDSDTDASNLETPRSESPDLILHDSGVSAARAAAAAAAAAAAAAAAASAGGKEAASGPNDS

SEQ ID NO: 23 - protein, 362 aa, Homo sapiens
MATAETALPSISTLTALGPFPDTQDDFLKWWRSEEAQDMGPGPPDPTEPPLHVKSEDQPGEEEDDERGADATWDLDLLLTNFSGPEPGGAPQTCALAPSEASGAQYPPPPETLGAYAGGPGLVAGLLGSEDHSGWVRPALRARAPDAFVGPALAPAPAPEPKALALQPVYPGPGAGSSGGYFPRTGLSVPAASGAPYGLLSGYPAMYPAPQYQGHFQLFRGLQGPAPGPATSPSFLSCLGPGTVGTGLGGTAEDPGVIAETAPSKRGRRSWARKRQAAHTCAHPGCGKSYTKSSHLKAHLRTHTGEKPYACTWEGCGWRFARSDELTRHYRKHTGQRPFRCQLCPRAFSRSDHLALHMKRHL

SEQ ID NO: 24 - protein, 355 aa, Homo sapiens
MALSEPILPSFSTFASPCRERGLQERWPRAEPESGGTDDDLNSVLDFILSMGLDGLGAEAAPEPPPPPPPPAFYYPEPGAPPPYSAPAGGLVSELLRPELDAPLGPALHGRFLLAPPGRLVKAEPPEADGGGGYGCAPGLTRGPRGLKREGAPGPAASCMRGPGGRPPPPPDTPPLSPDGPARLPAPGPRASFPPPFGGPGFGAPGPGLHYAPPAPPAFGLFDDAAAAAAALGLAPPAARGLLTPPASPLELLEAKPKRGRRSWPRKRTATHTCSYAGCGKTYTKSSHLKAHLRTHTGEKPYHCNWDGCGWKFARSDELTRHYRKHTGHRPFQCHLCDRAFSRSDHLALHMKRHM

SEQ ID NO: 25 - protein, 345 aa, Homo sapiens
MLMFDPVPVKQEAMDPVSVSYPSNYMESMKPNKYGVIYSTPLPEKFFQTPEGLSHGIQMEPVDLTVNKRSSPPSAGNSPSSLKFPSSHRRASPGLSMPSSSPPIKKYSPPSPGVQPFGVPLSMPPVMAAALSRHGIRSPGILPVIQPVVVQPVPFMYTSHLQQPLMVSLSEEMENSSSSMQVPVIESYEKPISQKKIKIEPGIEPQRTDYYPEEMSPPLMNSVSPPQALLQENHPSVIVQPGKRPLPVESPDTQRKRRIHRCDYDGCNKVYTKSSHLKAHRRTHTGEKPYKCTWEGCTWKFARSDELTRHFRKHTGIKPFQCPDCDRSFSRSDHLALHRKRHMLV

SEQ ID NO: 26 - protein, 513 aa, Homo sapiens
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQGQSRGFVARAGEPCVCWPHFGTHGMMLTPPSSPLELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 27 - protein, 457 aa, Homo sapiens
MATRVLSMSARLGPVPQPPAPQDEPVFAQLKPVLGAANPARDAALFPGEELKHAHHRPQAQPAPAQAPQPAQPPATGPRLPPEDLVQTRCEMEKYLTPQLPPVPIIPEHKKYRRDSASVVDQFFTDTEGLPYSINMNVFLPDITHLRTGLYKSQRPCVTHIKTEPVAIFSHQSETTAPPPAPTQALPEFTSIFSSHQTAAPEVNNIFIKQELPTPDLHLSVPTQQGHLYQLLNTPDLDMPSSTNQTAAMDTLNVSMSAAMAGLNTHTSAVPQTAVKQFQGMPPCTYTMPSQFLPQQATYFPPSPPSSEPGSPDRQAEMLQNLTPPPSYAATIASKLAIHNPNLPTTLPVNSQNIQPVRYNRRSNPDLEKRRIHYCDYPGCTKVYTKSSHLKAHLRTHTGEKPYKCTWEGCDWRFARSDELTRHYRKHTGAKPFQCGVCNRSFSRSDHLALHMKRHQN

SEQ ID NO: 28 - protein, 283 aa, Homo sapiens
MDVLPMCSIFQELQIVHETGYFSALPSLEEYWQQTCLELERYLQSEPCYVSASEIKFDSQEDLWTKIILAREKKEESELKISSSPPEDTLISPSFCYNLETNSLNSDVSSESSDSSEELSPTAKFTSDPIGEVLVSSGKLSSSVTSTPPSSPELSREPSQLWGCVPGELPSPGKVRSGTSGKPGDKGNGDASPDGRRRVHRCHFNGCRKVYTKSSHLKAHQRTHTGEKPYRCSWEGCEWRFARSDELTRHFRKHTGAKPFKCSHCDRCFSRSDHLALHMKRHL

SEQ ID NO: 29 - protein, 302 aa, Homo sapiens
MDVLASYSIFQELQLVHDTGYFSALPSLEETWQQTCLELERYLQTEPRRISETFGEDLDCFLHASPPPCIEESFRRLDPLLLPVEAAICEKSSAVDILLSRDKLLSETCLSLQPASSSLDSYTAVNQAQLNAVTSLTPPSSPELSRHLVKTSQTLSAVDGTVTLKLVAKKAALSSVKVGGVATAAAAVTAAGAVKSGQSDSDQGGLGAEACPENKKRVHRCQFNGCRKVYTKSSHLKAHQRTHTGEKPYKCSWEGCEWRFARSDELTRHYRKHTGAKPFKCNHCDRCFSRSDHLALHMKRHI

SEQ ID NO: 30 - protein, 359 aa, Homo sapiens
MVDMDKLINNLEVQLNSEGGSMQVFKQVTASVRNRDPPEIEYRSNMTSPTLLDANPMENPALFNDIKIEPPEELLASDFSLPQVEPVDLSFHKPKAPLQPASMLQAPIRPPKPQSSPQTLVVSTSTSDMSTSANIPTVLTPGSVLTSSQSTGSQQILHVIHTIPSVSLPNKMGGLKTIPVVVQSLPMVYTTLPADGGPAAITVPLIGGDGKNAGSVKVDPTSMSPLEIPSDSEESTIESGSSALQSLQGLQQEPAAMAQMQGEESLDLKRRRIHQCDFAGCSKVYTKSSHLKAHRRIHTGEKPYKCTWDGCSWKFARSDELTRHFRKHTGIKPFRCTDCNRSFSRSDHLSLHRRRHDTM

SEQ ID NO: 31 - protein, 244 aa, Homo sapiens
MSAAAYMDFVAAQCLVSISNRAAVPEHGVAPDAERLRLPEREVTKEHGDPGDTWKDYCTLVTIAKSLLDLNKYRPIQTPSVCSDSLESPDEDMGSDSDVTTESGSSPSHSPEERQDPGSAPSPLSLLHPGVAAKGKHASEKRHKCPYSGCGKVYGKSSHLKAHYRVHTGERPFPCTWPDCLKKFSRSDELTRHYRTHTGEKQFRCPLCEKRFMRSDHLTKHARRHTEFHPSMIKRSKKALANAL

SEQ ID NO: 32 - protein, 480 aa, Homo sapiens
MLNFGASLQQTAEERMEMISERPKESMYSWNKTAEKSDFEAVEALMSMSCSWKSDFKKYVENRPVTPVSDLSEEENLLPGTPDFHTIPAFCLTPPYSPSDFEPSQVSNLMAPAPSTVHFKSLSDTAKPHIAAPFKEEEKSPVSAPKLPKAQATSVIRHTADAQLCNHQTCPMKAASILNYQNNSFRRRTHLNVEAARKNIPCAAVSPNRSKCERNTVADVDEKASAALYDFSVPSSETVICRSQPAPVSPQQKSVLVSPPAVSAGGVPPMPVICQMVPLPANNPVVTTVVPSTPPSQPPAVCPPVVFMGTQVPKGAVMFVVPQPVVQSSKPPVVSPNGTRLSPIAPAPGFSPSAAKVTPQIDSSRIRSHICSHPGCGKTYFKSSHLKAHTRTHTGEKPFSCSWKGCERRFARSDELSRHRRTHTGEKKFACPMCDRRFMRSDHLTKHARRHLSAKKLPNWQMEVSKLNDIALPPTPAPTQ

SEQ ID NO: 33 - protein, 512 aa, Homo sapiens
MHTPDFAGPDDARAVDIMDICESILERKRHDSERSTCSILEQTDMEAVEALVCMSSWGQRSQKGDLLRIRPLTPVSDSGDVTTTVHMDAATPELPKDFHSLSTLCITPPQSPDLVEPSTRTPVSPQVTDSKACTATDVLQSSAVVARALSGGAERGLLGLEPVPSSPCRAKGTSVIRHTGESPAACFPTIQTPDCRLSDSREGEEQLLGHFETLQDTHLTDSLLSTNLVSCQPCLHKSGGLLLTDKGQQAGWPGAVQTCSPKNYENDLPRKTTPLISVSVPAPPVLCQMIPVTGQSSMLPAFLKPPPQLSVGTVRPILAQAAPAPQPVFVGPAVPQGAVMLVLPQGALPPPAPCAANVMAAGNTKLLPLAPAPVFITSSQNCVPQVDFSRRRNYVCSFPGCRKTYFKSSHLKAHLRTHTGEKPFNCSWDGCDKKFARSDELSRHRRTHTGEKKFVCPVCDRRFMRSDHLTKHARRHMTTKKIPGWQAEVGKLNRIASAESPGSPLVSMPASA

SEQ ID NO: 34 - protein, 402 aa, Homo sapiens
MNIHMKRKTIKNINTFENRMLMLDGMPAVRVKTELLESEQGSPNVHNYPDMEAVPLLLNNVKGEPPEDSLSVDHFQTQTEPVDLSINKARTSPTAVSSSPVSMTASASSPSSTSTSSSSSSRLASSPTVITSVSSASSSSTVLTPGPLVASASGVGGQQFLHIIHPVPPSSPMNLQSNKLSHVHRIPVVVQSVPVVYTAVRSPGNVNNTIVVPLLEDGRGHGKAQMDPRGLSPRQSKSDSDDDDLPNVTLDSVNETGSTALSIARAVQEVHPSPVSRVRGNRMNNQKFPCSISPFSIESTRRQRRSESPDSRKRRIHRCDFEGCNKVYTKSSHLKAHRRTHTGEKPYKCTWEGCTWKFARSDELTRHYRKHTGVKPFKCADCDRSFSRSDHLALHRRRHMLV

SEQ ID NO: 35 - protein, 288 aa, Homo sapiens
MAAAAYVDHFAAECLVSMSSRAVVHGPREGPESRPEGAAVAATPTLPRVEERRDGKDSASLFVVARILADLNQQAPAPAPAERREGAAARKARTPCRLPPPAPEPTSPGAEGAAAAPPSPAWSEPEPEAGLEPEREPGPAGSGEPGLRQRVRRGRSRADLESPQRKHKCHYAGCEKVYGKSSHLKAHLRTHTGERPFACSWQDCNKKFARSDELARHYRTHTGEKKFSCPICEKRFMRSDHLTKHARRHANFHPGMLQRRGGGSRTGSLSDYSRSDASSPTISPASSP

SEQ ID NO: 36 - protein, 323 aa, Homo sapiens
MSAAVACLDYFAAECLVSMSAGAVVHRRPPDPEGAGGAAGSEVGAAPPESALPGPGPPGPASVPQLPQVPAPSPGAGGAAPHLLAASVWADLRGSSGEGSWENSGEAPRASSGFSDPIPCSVQTPCSELAPASGAAAVCAPESSSDAPAVPSAPAAPGAPAASGGFSGGALGAGPAPAADQAPRRRSVTPAAKRHQCPFPGCTKAYYKSSHLKSHQRTHTGERPFSCDWLDCDKKFTRSDELARHYRTHTGEKRFSCPLCPKQFSRSDHLTKHARRHPTYHPDMIEYRGRRRTPRIDPPLTSEVESSASGSGPGPAPSFTTCL

SEQ ID NO: 37 - protein, 435 aa, Homo sapiens
MSEERDGPPRSRHRHGQASMVDHLLPVDENFSSPKCPVGYLGDRLVGRRAYHMLPSPVSEDDSDASSPCSCSSPDSQALCSCYGGGLGTESQDSILDFLLSQATLGSGGGSGSSIGASSGPVAWGPWRRAAAPVKGEHFCLPEFPLGDPDDVPRPFQPTLEEIEEFLEENMEPGVKEVPEGNSKDLDACSQLSAGPHKSHLHPGSSGRERCSPPPGGASAGGAQGPGGGPTPDGPIPVLLQIQPVPVKQESGTGPASPGQAPENVKVAQLLVNIQGQTFALVPQVVPSSNLNLPSKFVRIAPVPIAAKPVGSGPLGPGPAGLLMGQKFPKNPAAELIKMHKCTFPGCSKMYTKSSHLKAHLRRHTGEKPFACTWPGCGWRFSRSDELSRHRRSHSGVKPYQCPVCEKKFARSDHLSKHIKVHRFPRSSRSVRSVN

SEQ ID NO: 38 - protein, 252 aa, Homo sapiens
MSAAVACVDYFAADVLMAISSGAVVHRGRPGPEGAGPAAGLDVRAARREAASPGTPGPPPPPPAASGPGPGAAAAPHLLAASILADLRGGPGAAPGGASPASSSSAASSPSSGRAPGAAPSAAAKSHRCPFPDCAKAYYKSSHLKSHLRTHTGERPFACDWQGCDKKFARSDELARHHRTHTGEKRFSCPLCSKRFTRSDHLAKHARRHPGFHPDLLRRPGARSTSPSDSLPCSLAGSPAPSPAPSPAPAGL

SEQ ID NO: 39 - protein, 389 aa, Homo sapiens
MYGRPQAEMEQEAGELSRWQAAHQAAQDNENSAPILNMSSSSGSSGVHTSWNQGLPSIQHFPHSAEMLGSPLVSVEAPGQNVNEGGPQFSMPLPERGMSYCPQATLTPSRMIYCQRMSPPQQEMTIFSGPQLMPVGEPNIPRVARPFGGNLRMPPNGLPVSASTGIPIMSHTGNPPVPYPGLSTVPSDETLLGPTVPSTEAQAVLPSMAQMLPPQDAHDLGMPPAESQSLLVLGSQDSLVSQPDSQEGPFLPEQPGPAPQTVEKNSRPQEGTGRRGSSEARPYCCNYENCGKAYTKRSHLVSHQRKHTGERPYSCNWESCSWSFFRSDELRRHMRVHTRYRPYKCDQCSREFMRSDHLKQHQKTHRPGPSDPQANNNNGEQDSPPAAGP

SEQ ID NO: 40 - protein, 81 aa, Homo sapiens
CHIQGCGKVYGKTSHLRAHLRWHTGERPFMCTWSYCGKRFTRSDELQRHKRTHTGEKKFACPECPKRFMRSDHLSKHIKTH

SEQ ID NO: 41 - protein, 81 aa, Homo sapiens
CHIPDCGKTFRKTSLLRAHVRLHTGERPFVCNWFFCGKRFTRSDELQRHARTHTGDKRFECAQCQKRFMRSDHLTKHYKTH

SEQ ID NO: 42 - protein, 83 aa, Homo sapiens
HICHIPGCGKVYGKTSHLRAHLRWHSGERPFVCNWMYCGKRFTRSDELQRHRRTHTGEKKFVCPECSKRFMRSDHLAKHIKTH

SEQ ID NO: 43 - protein, 81 aa, Homo sapiens
CHIEGCGKVYGKTSHLRAHLRWHTGERPFICNWMFCGKRFTRSDELQRHRRTHTGEKRFECPECSKRFMRSDHLSKHVKTH

SEQ ID NO: 44 - protein, 81 aa, Homo sapiens
CHVPGCGKVYGKTSHLKAHLRWHTGERPFVCNWLFCGKSFTRSDELQRHLRTHTGEKRFACPECGKRFMRSDHLAKHVKTH

SEQ ID NO: 45 - protein, 81 aa, Homo sapiens
CHIPGCGKAYAKTSHLKAHLRWHSGDRPFVCNWLFCGKRFTRSDELQRHLQTHTGTKKFPCAVCSRVFMRSDHLAKHMKTH

SEQ ID NO: 46 - protein, 81 aa, Homo sapiens
CHIPGCGKVYGKASHLKAHLRWHTGERPFVCNWLFCGKRFTRSDELERHVRTHTREKKFTCLLCSKRFTRSDHLSKHQRTH

SEQ ID NO: 47 - protein, 81 aa, Homo sapiens
CHIPGCGKVYGKTSHLKAHLRWHTGERPFVCNWLFCGKRFTRSDELQRHLRTHTGEKRFACPVCNKRFMRSDHLSKHVKTH

SEQ ID NO: 48 - protein, 81 aa, Homo sapiens
CHIPGCGKVYGKTSHLKAHLRWHTGERPFVCNWLFCGKRFTRSDELQRHLRTHTGEKRFACPVCNKRFMRSDHLSKHIKTH

SEQ ID NO: 49 - protein, 83 aa, Homo sapiens
TCAHPGCGKSYTKSSHLKAHLRTHTGEKPYACTWEGCGWRFARSDELTRHYRKHTGQRPFRCQLCPRAFSRSDHLALHMKRHL

SEQ ID NO: 50 - protein, 83 aa, Homo sapiens
TCSYAGCGKTYTKSSHLKAHLRTHTGEKPYHCNWDGCGWKFARSDELTRHYRKHTGHRPFQCHLCDRAFSRSDHLALHMKRHM

SEQ ID NO: 51 - protein, 83 aa, Homo sapiens
RCDYDGCNKVYTKSSHLKAHRRTHTGEKPYKCTWEGCTWKFARSDELTRHFRKHTGIKPFQCPDCDRSFSRSDHLALHRKRHM

SEQ ID NO: 52 - protein, 83 aa, Homo sapiens
TCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 53 - protein, 83 aa, Homo sapiens
YCDYPGCTKVYTKSSHLKAHLRTHTGEKPYKCTWEGCDWRFARSDELTRHYRKHTGAKPFQCGVCNRSFSRSDHLALHMKRHQ

SEQ ID NO: 54 - protein, 83 aa, Homo sapiens
RCHFNGCRKVYTKSSHLKAHQRTHTGEKPYRCSWEGCEWRFARSDELTRHFRKHTGAKPFKCSHCDRCFSRSDHLALHMKRHL

SEQ ID NO: 55 - protein, 83 aa, Homo sapiens
RCQFNGCRKVYTKSSHLKAHQRTHTGEKPYKCSWEGCEWRFARSDELTRHYRKHTGAKPFKCNHCDRCFSRSDHLALHMKRHI

SEQ ID NO: 56 - protein, 83 aa, Homo sapiens
QCDFAGCSKVYTKSSHLKAHRRIHTGEKPYKCTWDGCSWKFARSDELTRHFRKHTGIKPFRCTDCNRSFSRSDHLSLHRRRHD

SEQ ID NO: 57 - protein, 83 aa, Homo sapiens
KCPYSGCGKVYGKSSHLKAHYRVHTGERPFPCTWPDCLKKFSRSDELTRHYRTHTGEKQFRCPLCEKRFMRSDHLTKHARRHT

SEQ ID NO: 58 - protein, 83 aa, Homo sapiens
ICSHPGCGKTYFKSSHLKAHTRTHTGEKPFSCSWKGCERRFARSDELSRHRRTHTGEKKFACPMCDRRFMRSDHLTKHARRHL

SEQ ID NO: 59 - protein, 83 aa, Homo sapiens
VCSFPGCRKTYFKSSHLKAHLRTHTGEKPFNCSWDGCDKKFARSDELSRHRRTHTGEKKFVCPVCDRRFMRSDHLTKHARRHM

SEQ ID NO: 60 - protein, 83 aa, Homo sapiens
RCDFEGCNKVYTKSSHLKAHRRTHTGEKPYKCTWEGCTWKFARSDELTRHYRKHTGVKPFKCADCDRSFSRSDHLALHRRRHM

SEQ ID NO: 61 - protein, 83 aa, Homo sapiens
KCHYAGCEKVYGKSSHLKAHLRTHTGERPFACSWQDCNKKFARSDELARHYRTHTGEKKFSCPICEKRFMRSDHLTKHARRHA

SEQ ID NO: 62 - protein, 83 aa, Homo sapiens
QCPFPGCTKAYYKSSHLKSHQRTHTGERPFSCDWLDCDKKFTRSDELARHYRTHTGEKRFSCPLCPKQFSRSDHLTKHARRHP

SEQ ID NO: 63 - protein, 83 aa, Homo sapiens
KCTFPGCSKMYTKSSHLKAHLRRHTGEKPFACTWPGCGWRFSRSDELSRHRRSHSGVKPYQCPVCEKKFARSDHLSKHIKVHR

SEQ ID NO: 64 - protein, 83 aa, Homo sapiens
RCPFPDCAKAYYKSSHLKSHLRTHTGERPFACDWQGCDKKFARSDELARHHRTHTGEKRFSCPLCSKRFTRSDHLAKHARRHP

SEQ ID NO: 65 - protein, 83 aa, Homo sapiens
CCNYENCGKAYTKRSHLVSHQRKHTGERPYSCNWESCSWSFFRSDELRRHMRVHTRYRPYKCDQCSREFMRSDHLKQHQKTHR

SEQ ID NO: 66 - protein, 473 aa, Homo sapiens
MAQHDFAPAWLNFPTPPSSTKSSLNFEKHSENFAWTENRYDVNRRRHNSSDGFDSAIGRPNGGNFGRKEKNGWRTHGRNGTENINHRGGYHGGSSRSRSSIFHAGKSQGLHENNIPDNETGRKEDKRERKQFEAEDFPSLNPEYEREPNHNKSLAAGVWEYPPNPKSRAPRMLVIKKGNTKDLQLSGFPVVGNLPSQPVKNGTGPSVYKGLVPKPAAPPTKPTQWKSQTKENKVGTSFPHESTFGVGNFNAFKSTAKNFSPSTNSVKECNRSNSSSPVDKLNQQPRLTKLTRMRTDKKSEFLKALKRDRVEEEHEDESRAGSEKDDDSFNLHNSNSTHQERDINRNFDENEIPQENGNASVISQQIIRSSTFPQTDVLSSSLEAEHRLLKEMGWQEDSENDETCAPLTEDEMREFQVISEQLQKNGLRKNGILKNGLICDFKFGPWKNSTFKPTTENDDTETSSSDTSDDDDV

SEQ ID NO: 67 - protein, 781 aa, Homo sapiens
MAHRPKRTFRQRAADSSDSDGAEESPAEPGAPRELPVPGSAEEEPPSGGGRAQVAGLPHRVRGPRGRGRVWASSRRATKAAPRADEGSESRTLDVSTDEEDKIHHSSESKDDQGLSSDSSSSLGEKELSSTVKIPDAAFIQAARRKRELARAQDDYISLDVQHTSSISGMKRESEDDPESEPDDHEKRIPFTLRPQTLRQRMAEESISRNEETSEESQEDEKQDTWEQQQMRKAVKIIEERDIDLSCGNGSSKVKKFDTSISFPPVNLEIIKKQLNTRLTLLQETHRSHLREYEKYVQDVKSSKSTIQNLESSSNQALNCKFYKSMKIYVENLIDCLNEKIINIQEIESSMHALLLKQAMTFMKRRQDELKHESTYLQQLSRKDETSTSGNFSVDEKTQWILEEIESRRTKRRQARVLSGNCNHQEGTSSDDELPSAEMIDFQKSQGDILQKQKKVFEEVQDDFCNIQNILLKFQQWREKFPDSYYEAFISLCIPKLLNPLIRVQLIDWNPLKLESTGLKEMPWFKSVEEFMDSSVEDSKKESSSDKKVLSAIINKTIIPRLTDFVEFLWDPLSTSQTTSLITHCRVILEEHSTCENEVSKSRQDLLKSIVSRMKKAVEDDVFIPLYPKSAVENKTSPHSKFQERQFWSGLKLFRNILLWNGLLTDDTLQELGLGKLLNRYLIIALLNATPGPDVVKKCNQVAACLPEKWFENSAMRTSIPQLENFIQFLLQSAHKLSRSEFRDEVEEIILILVKIKALNQAESFIGEHHLDHLKSLIKED

SEQ ID NO: 68 - protein, 474 aa, Homo sapiens
MAQHDFVPAWLNFSTPQSAKSPTATFEKHGEHLPRGEGRFGVSRRRHNSSDGFFNNGPLRTAGDSWHQPSLFRHDSVDSGVSKGAYAGITGNPSGWHSSSRGHDGMSQRSGGGTGNHRHWNGSFHSRKGCAFQEKPPMEIREEKKEDKVEKLQFEEEDFPSLNPEAGKQHQPCRPIGTPSGVWENPPSAKQPSKMLVIKKVSKEDPAAAFSAAFTSPGSHHANGNKLSSVVPSVYKNLVPKPVPPPSKPNAWKANRMEHKSGSLSSSRESAFTSPISVTKPVVLASGAALSSPKESPSSTTPPIEISSSRLTKLTRRTTDRKSEFLKTLKDDRNGDFSENRDCDKLEDLEDNSTPEPKENGEEGCHQNGLALPVVEEGEVLSHSLEAEHRLLKAMGWQEYPENDENCLPLTEDELKEFHMKTEQLRRNGFGKNGFLQSRSSSLFSPWRSTCKAEFEDSDTETSSSETSDDDAWK

SEQ ID NO: 69 - protein, 917 aa, Homo sapiens
MFRKARRVNVRKRNDSEEEERERDEEQEPPPLLPPPGTGEEAGPGGGDRAPGGESLLGPGPSPPSALTPGLGAEAGGGFPGGAEPGNGLKPRKRPRENKEVPRASLLSFQDEEEENEEVFKVKKSSYSKKIVKLLKKEYKEDLEKSKIKTELNSSAESEQPLDKTGHVKDTNQEDGVIISEHGEDEMDMESEKEEEKPKTGGAFSNALSSLNVLRPGEIPDAAFIHAARKKRQMARELGDFTPHDNEPGKGRLVREDENDASDDEDDDEKRRIVFSVKEKSQRQKIAEEIGIEGSDDDALVTGEQDEELSRWEQEQIRKGINIPQVQASQPAEVNMYYQNTYQTMPYGSSYGIPYSYTAYGSSDAKSQKTDNTVPFKTPSNEMTPVTIDLVKKQLKDRLDSMKELHKTNRQQHEKHLQSRVDSTRAIERLEGSSGGIGERYKFLQEMRGYVQDLLECFSEKVPLINELESAIHQLYKQRASRLVQRRQDDIKDESSEFSSHSNKALMAPNLDSFGRDRALYQEHAKRRIAEREARRTRRRQAREQTGKMADHLEGLSSDDEETSTDITNFNLEKDRISKESGKVFEDVLESFYSIDCIKSQFEAWRSKYYTSYKDAYIGLCLPKLFNPLIRLQLLTWTPLEAKCRDFENMLWFESLLFYGCEEREQEKDDVDVALLPTIVEKVILPKLTVIAENMWDPFSTTQTSRMVGITLKLINGYPSVVNAENKNTQVYLKALLLRMRRTLDDDVFMPLYPKNVLENKNSGPYLFFQRQFWSSVKLLGNFLQWYGIFSNKTLQELSIDGLLNRYILMAFQNSEYGDDSIKKAQNVINCFPKQWFMNLKGERTISQLENFCRYLVHLADTIYRNSIGCSDVEKRNARENIKQIVKLLASVRALDHAMSVASDHNVKEFKSLIEGK

SEQ ID NO: 70 - protein, 895 aa, Homo sapiens
MKIGSGFLSGGGGTGSSGGSGSGGGGSGGGGGGGSSGRRAEMEPTFPQGMVMFNHRLPPVTSFTRPAGSAAPPPQCVLSSSTSAAPAAEPPPPPAPDMTFKKEPAASAAAFPSQRTSWGFLQSLVSIKQEKPADPEEQQSHHHHHHHHYGGLFAGAEERSPGLGGGEGGSHGVIQDLSILHQHVQQQPAQHHRDVLLSSSSRTDDHHGTEEPKQDTNVKKAKRPKPESQGIKAKRKPSASSKPSLVGDGEGAILSPSQKPHICDHCSAAFRSSYHLRRHVLIHTGERPFQCSQCSMGFIQKYLLQRHEKIHSREKPFGCDQCSMKFIQKYHMERHKRTHSGEKPYKCDTCQQYFSRTDRLLKHRRTCGEVIVKGATSAEPGSSNHTNMGNLAVLSQGNTSSSRRKTKSKSIAIENKEQKTGKTNESQISNNINMQSYSVEMPTVSSSGGIIGTGIDELQKRVPKLIFKKGSRKNTDKNYLNFVSPLPDIVGQKSLSGKPSGSLGIVSNNSVETIGLLQSTSGKQGQISSNYDDAMQFSKKRRYLPTASSNSAFSINVGHMVSQQSVIQSAGVSVLDNEAPLSLIDSSALNAEIKSCHDKSGIPDEVLQSILDQYSNKSESQKEDPFNIAEPRVDLHTSGEHSELVQEENLSPGTQTPSNDKASMLQEYSKYLQQAFEKSTNASFTLGHGFQFVSLSSPLHNHTLFPEKQIYTTSPLECGFGQSVTSVLPSSLPKPPFGMLFGSQPGLYLSALDATHQQLTPSQELDDLIDSQKNLETSSAFQSSSQKLTSQKEQKNLESSTGFQIPSQELASQIDPQKDIEPRTTYQIENFAQAFGSQFKSGSRVPMTFITNSNGEVDHRVRTSVSDFSGYTNMMSDVSEPCSTRVKTPTSQSYR

SEQ ID NO: 71 - protein, 450 aa, Homo sapiens
MNLETGSRGSEFGMSAVSCGNGKLRQWLIDQIDSGKYPGLVWENEEKSVFRIPWKHAGKQDYNREEDAALFKAWALFKGKFREGIDKPDPPTWKTRLRCALNKSNDFEELVERSQLDISDPYKVYRIVPEGAKKGAKQLTLDDTQMAMGHPYPMTAPYGSLPAQQVHNYMMPPHDRSWRDYAPDQSHPEIPYQCPVTFGPRGHHWQGPSCENGCQVTGTFYACAPPESQAPGIPIEPSIRSAEALALSDCRLHICLYYRDILVKELTTTSPEGCRISHGHTYDVSNLDQVLFPYPDDNGQRKNIEKLLSHLERGLVLWMAPDGLYAKRLCQSRIYWDGPLALCSDRPNKLERDQTCKLFDTQQFLSELQVFAHHGRPAPRFQVTLCFGEEFPDPQRQRKLITAHVEPLLARQLYYFAQQNTGHFLRGYELPEHVTTPDYHRSLRHSSIQE

SEQ ID NO: 72 - protein, 1394 aa, synthetic construct
MSDQDHSMDEMTAVVKIEKGVGGNNGGNGNGGGAFSQARSSSTGSSSSTGGGGQESQPSPLALLAATCSRIESPNENSNNSQGPSQSGGTGELDLTATQLSQGANGWQIISSSSGATPTSKEQSGSSTNGSNGSESSKNRTVSGGQYVVAAAPNLQNQQVLTGLPGVMPNIQYQVIPQFQTVDGQQLQFAATGAQVQQDGSGQIQIIPGANQQIITNRGSGGNIIAAMPNLLQQAVPLQGLANNVLSGQTQYVTNVPVALNGNITLLPVNSVSAATLTPSSQAVTISSSGSQESGSQPVTSGTTISSASLVSSQASSSSFFTNANSYSTTTTTSNMGIMNFTTSGSSGTNSQGQTPQRVSGLQGSDALNIQQNQTSGGSLQAGQQKEGEQNQQTQQQQILIQPQLVQGGQALQALQAAPLSGQTFTTQAISQETLQNLQLQAVPNSGPIIIRTPTVGPNGQVSWQTLQLQNLQVQNPQAQTITLAPMQGVSLGQTSSSNTTLTPIASAASIPAGTVTVNAAQLSSMPGLQTINLSALGTSGIQVHPIQGLPLAIANAPGDHGAQLGLHGAGGDGIHDDTAGGEEGENSPDAQPQAGRRTRREACTCPYCKDSEGRGSGDPGKKKQHICHIQGCGKVYGKTSHLRAHLRWHTGERPFMCTWSYCGKRFTRSDELQRHKRTHTGEKKFACPECPKRFMRSDHLSKHIKTHQNKKGGPGVALSVGTLPLDSGAGSEGSGTATPSALITTNMVAMEAICPEGIARLANSGINVMQVADLQSINISGNGFGGGGSGGGGSGGGGSGGGGSGGGGSGSSYPFALASEPDEEIQETLDEDDDSEEDDVEDVSNDLEEELTDSDDDGSESEKSDFVIEQPGSSLASNRILTLPQRTIRGKNKHCWSTSKSTRRSRVSALNIVRSQRGPTRMCRNIYDPLLCFKLFFTDEIISEIVKHTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRQTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFLKELYMSLTSSFMRKRLEAPTLKRYLRDNISNILPNEVPGTSDDSTEEPVMKKRTYCTYCPSKIRRKANASCKKCKKVICREHNIDMCQSCFG

SEQ ID NO: 73 - protein, 690 aa, synthetic construct
CHIQGCGKVYGKTSHLRAHLRWHTGERPFMCTWSYCGKRFTRSDELQRHKRTHTGEKKFACPECPKRFMRSDHLSKHIKTHGGGGSGGGGSGGGGSGGGGSGGGGSGSSYPFALASEPDEEIQETLDEDDDSEEDDVEDVSNDLEEELTDSDDDGSESEKSDFVIEQPGSSLASNRILTLPQRTIRGKNKHCWSTSKSTRRSRVSALNIVRSQRGPTRMCRNIYDPLLCFKLFFTDEIISEIVKHTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRQTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFLKELYMSLTSSFMRKRLEAPTLKRYLRDNISNILPNEVPGTSDDSTEEPVMKKRTYCTYCPSKIRRKANASCKKCKKVICREHNIDMCQSCFG

SEQ ID NO: 74 - protein, 1083 aa, synthetic construct
MMAQHDFAPAWLNFPTPPSSTKSSLNFEKHSENFAWTENRYDVNRRRHNSSDGFDSAIGRPNGGNFGRKEKNGWRTHGRNGTENINHRGGYHGGSSRSRSSIFHAGKSQGLHENNIPDNETGRKEDKRERKQFEAEDFPSLNPEYEREPNHNKSLAAGVWEYPPNPKSRAPRMLVIKKGNTKDLQLSGFPVVGNLPSQPVKNGTGPSVYKGLVPKPAAPPTKPTQWKSQTKENKVGTSFPHESTFGVGNFNAFKSTAKNFSPSTNSVKECNRSNSSSPVDKLNQQPRLTKLTRMRTDKKSEFLKALKRDRVEEEHEDESRAGSEKDDDSFNLHNSNSTHQERDINRNFDENEIPQENGNASVISQQIIRSSTFPQTDVLSSSLEAEHRLLKEMGWQEDSENDETCAPLTEDEMREFQVISEQLQKNGLRKNGILKNGLICDFKFGPWKNSTFKPTTENDDTETSSSDTSDDDDVGGGGSGGGGSGGGGSGGGGSGGGGSGSSYPFALASEPDEEIQETLDEDDDSEEDDVEDVSNDLEEELTDSDDDGSESEKSDFVIEQPGSSLASNRILTLPQRTIRGKNKHCWSTSKSTRRSRVSALNIVRSQRGPTRMCRNIYDPLLCFKLFFTDEIISEIVKHTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRQTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFLKELYMSLTSSFMRKRLEAPTLKRYLRDNISNILPNEVPGTSDDSTEEPVMKKRTYCTYCPSKIRRKANASCKKCKKVICREHNIDMCQSCFG

SEQ ID NO: 75 - protein, 788 aa, synthetic construct
MVPQLQNIVSTVNLGCKLDLKTIALRARNAEYNPKRFAAVIMRIREPRTTALIFSSGKMVCTGAKSEEQSRLAARKYARVVQKLGFPAKFLDFKIQNMVGSCDVKFPIRLEGLVLTHQQFSSYEPELFPGLIYRMIKPRIVLLIFVSGKVVLTGAKVRAEIYEAFENIYPILKGFRKTTGGGGSGGGGSGGGGSGGGGSGGGGSGSSYPFALASEPDEEIQETLDEDDDSEEDDVEDVSNDLEEELTDSDDDGSESEKSDFVIEQPGSSLASNRILTLPQRTIRGKNKHCWSTSKSTRRSRVSALNIVRSQRGPTRMCRNIYDPLLCFKLFFTDEIISEIVKHTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRQTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFLKELYMSLTSSFMRKRLEAPTLKRYLRDNISNILPNEVPGTSDDSTEEPVMKKRTYCTYCPSKIRRKANASCKKCKKVICREHNIDMCQSCFG

SEQ ID NO: 76 - protein, 1223 aa, synthetic construct
MMAAQAAAAAQAAAAQAAQAEAADSWYLALLGFAEHFRTSSPPKIRLCVHCLQAVFPFKPPQRIEARTHLQLGSVLYHHTKNSEQARSHLEKAWLISQQIPQFEDVKFEAASLLSELYCQENSVDAAKPLLRKAIQISQQTPYWHCRLLFQLAQLHTLEKDLVSACDLLGVGAEYARVVGSEYTRALFLLSKGMLLLMERKLQEVHPLLTLCGQIVENWQGNPIQKESLRVFFLVLQVTHYLDAGQVKSVKPCLKQLQQCIQTISTLHDDEILPSNPADLFHWLPKEHMCVLVYLVTVMHSMQAGYLEKAQKYTDKALMQLEKLKMLDCSPILSSFQVILLEHIIMCRLVTGHKATALQEISQVCQLCQQSPRLFSNHAAQLHTLLGLYCVSVNCMDNAEAQFTTALRLTNHQELWAFIVTNLASVYIREGNRHQEVLYSLLERINPDHSFPVSSHCLRAAAFYVRGLFSFFQGRYNEAKRFLRETLKMSNAEDLNRLTACSLVLLGHIFYVLGNHRESNNMVVPAMQLASKIPDMSVQLWSSALLRDLNKACGNAMDAHEAAQMHQNFSQQLLQDHIEACSLPEHNLITWTDGPPPVQFQAQNGPNTSLASLLGGGGSGGGGSGGGGSGGGGSGGGGSGSSYPFALASEPDEEIQETLDEDDDSEEDDVEDVSNDLEEELTDSDDDGSESEKSDFVIEQPGSSLASNRILTLPQRTIRGKNKHCWSTSKSTRRSRVSALNIVRSQRGPTRMCRNIYDPLLCFKLFFTDEIISEIVKHTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRQTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFLKELYMSLTSSFMRKRLEAPTLKRYLRDNISNILPNEVPGTSDDSTEEPVMKKRTYCTYCPSKIRRKANASCKKCKKVICREHNIDMCQSCFG

SEQ ID NO: 77 - protein, 1640 aa, synthetic construct
MSDQDHSMDEMTAVVKIEKGVGGNNGGNGNGGGAFSQARSSSTGSSSSTGGGGQESQPSPLALLAATCSRIESPNENSNNSQGPSQSGGTGELDLTATQLSQGANGWQIISSSSGATPTSKEQSGSSTNGSNGSESSKNRTVSGGQYVVAAAPNLQNQQVLTGLPGVMPNIQYQVIPQFQTVDGQQLQFAATGAQVQQDGSGQIQIIPGANQQIITNRGSGGNIIAAMPNLLQQAVPLQGLANNVLSGQTQYVTNVPVALNGNITLLPVNSVSAATLTPSSQAVTISSSGSQESGSQPVTSGTTISSASLVSSQASSSSFFTNANSYSTTTTTSNMGIMNFTTSGSSGTNSQGQTPQRVSGLQGSDALNIQQNQTSGGSLQAGQQKEGEQNQQTQQQQILIQPQLVQGGQALQALQAAPLSGQTFTTQAISQETLQNLQLQAVPNSGPIIIRTPTVGPNGQVSWQTLQLQNLQVQNPQAQTITLAPMQGVSLGQTSSSNTTLTPIASAASIPAGTVTVNAAQLSSMPGLQTINLSALGTSGIQVHPIQGLPLAIANAPGDHGAQLGLHGAGGDGIHDDTAGGEEGENSPDAQPQAGRRTRREACTCPYCKDSEGRGSGDPGKKKQHICHIQGCGKVYGKTSHLRAHLRWHTGERPFMCTWSYCGKRFTRSDELQRHKRTHTGEKKFACPECPKRFMRSDHLSKHIKTHQNKKGGPGVALSVGTLPLDSGAGSEGSGTATPSALITTNMVAMEAICPEGIARLANSGINVMQVADLQSINISGNGFGGGGSGGGGSGGGGSGGGGSGGGGSNKGRKRTLRSRSRPLAHKRGTRAVEPEDPRPDSQGQRGGPKPGRYEPYDWDSDSDGGHRDGGGHGRVGRKTRAGRRDKAREPDAAAVEPVGPRHDSEGQRGAPKPGRYEPYDWDSDSDGGDGGGHGPPRDEALEPDAAAVEPEGPRHDSEGQGGGDGDRGGHGQVGRETLEDRRSDPPRDEALEPDAAAVEPEGPRHDSEGQGGGDGDRGGHGQVGRETLEDRRSDPPRDEAHEPDDGAVEPGGPQHELEGQGRGDEPSDWDLYECETDVDELDGSQSETEEQHRGPRSSGEEDVEEAVEEDAEEDIEEEPAAESASVYPDTFSSKNGEIKWSANERDRSSVRASPPHQASGPTTEAREGAQDILSSFLLFFTPNVERLILIATNRHSADKAHALKTPKPMDEIELRAYVGLLILAGVYRSRGEALVSLWEPECGRPIFGATMSLRSFYQRSATLRFDDRSTRASRSASDRLAAVREVWEEWCDRLPRLYDIGPEVTVDERMVPFKGRCSFRQYIPSKPTKYGLKLWVACDARTSYAWRVQPYMGKLASGARETNLASRVVSDLTRGLEHRNVTCDNFFTSYALATELLKRNITLLGTIKSNKPELPPELASARRREALSSRFAFTDCAAALSYVPKKNKNVLLLSTKHSRVEICHERRDKKPEMILDYNKTKGGVDNLDKLLATYSCRRMTKRWPLAMFHNMIDMSAYNAYVIWREMHPDWMPWNRNRRRIFLEQLGKDLVRPLVLRRKRVPRARPAYDLMVSMQEAGGTSARSEGKRKRCQICPRSKDTKTLISCKSCGKSICRGCTVPFCPLCSERQGRDGDTDASQ

SEQ ID NO: 78 - protein, 936 aa, synthetic construct
CHIQGCGKVYGKTSHLRAHLRWHTGERPFMCTWSYCGKRFTRSDELQRHKRTHTGEKKFACPECPKRFMRSDHLSKHIKTHGGGGSGGGGSGGGGSGGGGSGGGGSNKGRKRTLRSRSRPLAHKRGTRAVEPEDPRPDSQGQRGGPKPGRYEPYDWDSDSDGGHRDGGGHGRVGRKTRAGRRDKAREPDAAAVEPVGPRHDSEGQRGAPKPGRYEPYDWDSDSDGGDGGGHGPPRDEALEPDAAAVEPEGPRHDSEGQGGGDGDRGGHGQVGRETLEDRRSDPPRDEALEPDAAAVEPEGPRHDSEGQGGGDGDRGGHGQVGRETLEDRRSDPPRDEAHEPDDGAVEPGGPQHELEGQGRGDEPSDWDLYECETDVDELDGSQSETEEQHRGPRSSGEEDVEEAVEEDAEEDIEEEPAAESASVYPDTFSSKNGEIKWSANERDRSSVRASPPHQASGPTTEAREGAQDILSSFLLFFTPNVERLILIATNRHSADKAHALKTPKPMDEIELRAYVGLLILAGVYRSRGEALVSLWEPECGRPIFGATMSLRSFYQRSATLRFDDRSTRASRSASDRLAAVREVWEEWCDRLPRLYDIGPEVTVDERMVPFKGRCSFRQYIPSKPTKYGLKLWVACDARTSYAWRVQPYMGKLASGARETNLASRVVSDLTRGLEHRNVTCDNFFTSYALATELLKRNITLLGTIKSNKPELPPELASARRREALSSRFAFTDCAAALSYVPKKNKNVLLLSTKHSRVEICHERRDKKPEMILDYNKTKGGVDNLDKLLATYSCRRMTKRWPLAMFHNMIDMSAYNAYVIWREMHPDWMPWNRNRRRIFLEQLGKDLVRPLVLRRKRVPRARPAYDLMVSMQEAGGTSARSEGKRKRCQICPRSKDTKTLISCKSCGKSICRGCTVPFCPLCSERQGRDGDTDASQ

SEQ ID NO: 79 - protein, 1329 aa, synthetic construct
MMAQHDFAPAWLNFPTPPSSTKSSLNFEKHSENFAWTENRYDVNRRRHNSSDGFDSAIGRPNGGNFGRKEKNGWRTHGRNGTENINHRGGYHGGSSRSRSSIFHAGKSQGLHENNIPDNETGRKEDKRERKQFEAEDFPSLNPEYEREPNHNKSLAAGVWEYPPNPKSRAPRMLVIKKGNTKDLQLSGFPVVGNLPSQPVKNGTGPSVYKGLVPKPAAPPTKPTQWKSQTKENKVGTSFPHESTFGVGNFNAFKSTAKNFSPSTNSVKECNRSNSSSPVDKLNQQPRLTKLTRMRTDKKSEFLKALKRDRVEEEHEDESRAGSEKDDDSFNLHNSNSTHQERDINRNFDENEIPQENGNASVISQQIIRSSTFPQTDVLSSSLEAEHRLLKEMGWQEDSENDETCAPLTEDEMREFQVISEQLQKNGLRKNGILKNGLICDFKFGPWKNSTFKPTTENDDTETSSSDTSDDDDVGGGGSGGGGSGGGGSGGGGSGGGGSNKGRKRTLRSRSRPLAHKRGTRAVEPEDPRPDSQGQRGGPKPGRYEPYDWDSDSDGGHRDGGGHGRVGRKTRAGRRDKAREPDAAAVEPVGPRHDSEGQRGAPKPGRYEPYDWDSDSDGGDGGGHGPPRDEALEPDAAAVEPEGPRHDSEGQGGGDGDRGGHGQVGRETLEDRRSDPPRDEALEPDAAAVEPEGPRHDSEGQGGGDGDRGGHGQVGRETLEDRRSDPPRDEAHEPDDGAVEPGGPQHELEGQGRGDEPSDWDLYECETDVDELDGSQSETEEQHRGPRSSGEEDVEEAVEEDAEEDIEEEPAAESASVYPDTFSSKNGEIKWSANERDRSSVRASPPHQASGPTTEAREGAQDILSSFLLFFTPNVERLILIATNRHSADKAHALKTPKPMDEIELRAYVGLLILAGVYRSRGEALVSLWEPECGRPIFGATMSLRSFYQRSATLRFDDRSTRASRSASDRLAAVREVWEEWCDRLPRLYDIGPEVTVDERMVPFKGRCSFRQYIPSKPTKYGLKLWVACDARTSYAWRVQPYMGKLASGARETNLASRVVSDLTRGLEHRNVTCDNFFTSYALATELLKRNITLLGTIKSNKPELPPELASARRREALSSRFAFTDCAAALSYVPKKNKNVLLLSTKHSRVEICHERRDKKPEMILDYNKTKGGVDNLDKLLATYSCRRMTKRWPLAMFHNMIDMSAYNAYVIWREMHPDWMPWNRNRRRIFLEQLGKDLVRPLVLRRKRVPRARPAYDLMVSMQEAGGTSARSEGKRKRCQICPRSKDTKTLISCKSCGKSICRGCTVPFCPLCSERQGRDGDTDASQ

SEQ ID NO: 80 - protein, 1034 aa, synthetic construct
MVPQLQNIVSTVNLGCKLDLKTIALRARNAEYNPKRFAAVIMRIREPRTTALIFSSGKMVCTGAKSEEQSRLAARKYARVVQKLGFPAKFLDFKIQNMVGSCDVKFPIRLEGLVLTHQQFSSYEPELFPGLIYRMIKPRIVLLIFVSGKVVLTGAKVRAEIYEAFENIYPILKGFRKTTGGGGSGGGGSGGGGSGGGGSGGGGSNKGRKRTLRSRSRPLAHKRGTRAVEPEDPRPDSQGQRGGPKPGRYEPYDWDSDSDGGHRDGGGHGRVGRKTRAGRRDKAREPDAAAVEPVGPRHDSEGQRGAPKPGRYEPYDWDSDSDGGDGGGHGPPRDEALEPDAAAVEPEGPRHDSEGQGGGDGDRGGHGQVGRETLEDRRSDPPRDEALEPDAAAVEPEGPRHDSEGQGGGDGDRGGHGQVGRETLEDRRSDPPRDEAHEPDDGAVEPGGPQHELEGQGRGDEPSDWDLYECETDVDELDGSQSETEEQHRGPRSSGEEDVEEAVEEDAEEDIEEEPAAESASVYPDTFSSKNGEIKWSANERDRSSVRASPPHQASGPTTEAREGAQDILSSFLLFFTPNVERLILIATNRHSADKAHALKTPKPMDEIELRAYVGLLILAGVYRSRGEALVSLWEPECGRPIFGATMSLRSFYQRSATLRFDDRSTRASRSASDRLAAVREVWEEWCDRLPRLYDIGPEVTVDERMVPFKGRCSFRQYIPSKPTKYGLKLWVACDARTSYAWRVQPYMGKLASGARETNLASRVVSDLTRGLEHRNVTCDNFFTSYALATELLKRNITLLGTIKSNKPELPPELASARRREALSSRFAFTDCAAALSYVPKKNKNVLLLSTKHSRVEICHERRDKKPEMILDYNKTKGGVDNLDKLLATYSCRRMTKRWPLAMFHNMIDMSAYNAYVIWREMHPDWMPWNRNRRRIFLEQLGKDLVRPLVLRRKRVPRARPAYDLMVSMQEAGGTSARSEGKRKRCQICPRSKDTKTLISCKSCGKSICRGCTVPFCPLCSERQGRDGDTDASQ

SEQ ID NO: 81 - protein, 1469 aa, synthetic construct
MMAAQAAAAAQAAAAQAAQAEAADSWYLALLGFAEHFRTSSPPKIRLCVHCLQAVFPFKPPQRIEARTHLQLGSVLYHHTKNSEQARSHLEKAWLISQQIPQFEDVKFEAASLLSELYCQENSVDAAKPLLRKAIQISQQTPYWHCRLLFQLAQLHTLEKDLVSACDLLGVGAEYARVVGSEYTRALFLLSKGMLLLMERKLQEVHPLLTLCGQIVENWQGNPIQKESLRVFFLVLQVTHYLDAGQVKSVKPCLKQLQQCIQTISTLHDDEILPSNPADLFHWLPKEHMCVLVYLVTVMHSMQAGYLEKAQKYTDKALMQLEKLKMLDCSPILSSFQVILLEHIIMCRLVTGHKATALQEISQVCQLCQQSPRLFSNHAAQLHTLLGLYCVSVNCMDNAEAQFTTALRLTNHQELWAFIVTNLASVYIREGNRHQEVLYSLLERINPDHSFPVSSHCLRAAAFYVRGLFSFFQGRYNEAKRFLRETLKMSNAEDLNRLTACSLVLLGHIFYVLGNHRESNNMVVPAMQLASKIPDMSVQLWSSALLRDLNKACGNAMDAHEAAQMHQNFSQQLLQDHIEACSLPEHNLITWTDGPPPVQFQAQNGPNTSLASLLGGGGSGGGGSGGGGSGGGGSGGGGSNKGRKRTLRSRSRPLAHKRGTRAVEPEDPRPDSQGQRGGPKPGRYEPYDWDSDSDGGHRDGGGHGRVGRKTRAGRRDKAREPDAAAVEPVGPRHDSEGQRGAPKPGRYEPYDWDSDSDGGDGGGHGPPRDEALEPDAAAVEPEGPRHDSEGQGGGDGDRGGHGQVGRETLEDRRSDPPRDEALEPDAAAVEPEGPRHDSEGQGGGDGDRGGHGQVGRETLEDRRSDPPRDEAHEPDDGAVEPGGPQHELEGQGRGDEPSDWDLYECETDVDELDGSQSETEEQHRGPRSSGEEDVEEAVEEDAEEDIEEEPAAESASVYPDTFSSKNGEIKWSANERDRSSVRASPPHQASGPTTEAREGAQDILSSFLLFFTPNVERLILIATNRHSADKAHALKTPKPMDEIELRAYVGLLILAGVYRSRGEALVSLWEPECGRPIFGATMSLRSFYQRSATLRFDDRSTRASRSASDRLAAVREVWEEWCDRLPRLYDIGPEVTVDERMVPFKGRCSFRQYIPSKPTKYGLKLWVACDARTSYAWRVQPYMGKLASGARETNLASRVVSDLTRGLEHRNVTCDNFFTSYALATELLKRNITLLGTIKSNKPELPPELASARRREALSSRFAFTDCAAALSYVPKKNKNVLLLSTKHSRVEICHERRDKKPEMILDYNKTKGGVDNLDKLLATYSCRRMTKRWPLAMFHNMIDMSAYNAYVIWREMHPDWMPWNRNRRRIFLEQLGKDLVRPLVLRRKRVPRARPAYDLMVSMQEAGGTSARSEGKRKRCQICPRSKDTKTLISCKSCGKSICRGCTVPFCPLCSERQGRDGDTDASQ

SEQ ID NO: 82 - protein, 612 aa, Musca domestica
MQKMDNLEVKAKINQGLYKITPRHKGTSFIWNVLADIQKEDDTLVEGWVFCRKCEKVLKYTTRQTSNLCRHKCCASLKQSRELKTVSADCKKEAIEKCAQWVVRDCRPFSAVSGSGFIDMIKFFIKVGAEYGEHVNVEELLPSPITLSRKVTSDAKEKKALISREIKSAVEKDGASATIDLWTDNYIKRNFLGVTLHYHENNELRDLILGLKSLDFERSTAENIYKKLKAIFLQFNVEDLSSIKFVTDRGANVVKSLANNIRINCSSHLLSNVLENSFEETPELNVPILACKNIVKYFKKANLQHRLRSSLKSECPTRWNSTYTMLRSILDNWESVIQILSEAGETQRIVHINKSIIQTMVNILDGFERIFKELQTCSSPSLCFVVPSILKVKEICSPDVGDVADIAKLKVNIIKNVRIIWEENLSIWHYTAFFFYPPALHMQQEKVAQIKEFCLSKMEDLELINRMSSFNELSATQLNQSDSNSHNSIDLTSHSKDISTTSFFFPQLTQNNSREPPVCPSDEFEFYRKEIVILSEDFKVMEWWNLNSKKYPKLSKLALSLLSIPASSAASERTFSLAGNIITEKRNRIGQQTVDSLLFLNSFYKNFCKLDI

SEQ ID NO: 83 - protein, 636 aa, Tribolium castaneum; identical to SEQ ID NO: 4

SEQ ID NO: 84 - DNA, 35 bp, Trichoplusia ni
  source 1..35: mol_type = genomic DNA
ccctagaaagatagtctgcgtaaaattgacgcatg

SEQ ID NO: 85 - DNA, 63 bp, Trichoplusia ni
  source 1..63: mol_type = genomic DNA
catgcgtcaattttacgcatgattatctttaacgtacgtcacaatatgattatctttctaggg

SEQ ID NO: 86 - DNA, 534 bp, Oryzias latipes
  source 1..534: mol_type = genomic DNA
ttaaccctcccactcccctaagactggtttcaggatcccctacgctgtaatgaagggtccacatgaccccaaacttactctgacaagttctccaagtgatcccaccttacccttaggctgtcagaatgttcactcgattgtcacacagttagggaatacagcccttacattgaaaagggtacacagagcggccttggatcttctgacagcctaagagtaaggtgggatcacttggagaacttgtcagagtaaggttggggtcatgtggacccttcattacagcgtaggggatcctgaaaccagccttaggggagtgggagggttatatatactttatacgattttactcattgaaatacatgtttccttttaaatgtatgacaataacccttgcgctatcctagagaaaaaagaaataaattggattagataaaggaacgggggggcttattttaaccctttggctgtcctagagggggaacaaaaaggcttggattgaaggaagggaatgggggggtcattttaaaaacgcac

SEQ ID NO: 87 - DNA, 210 bp, Oryzias latipes
  source 1..210: mol_type = genomic DNA
caaatttttggggtgaatgaccactcttcagtggtcagggaccaaaccaggatgagggggtgcgtttttaaaatgacccccccattcccttccttcaatccaagcctttttgttccccctctaggacagccaaagggttaaaataagcccccccgttcctttatctaatccaatttatttcttttttctctaggatagcgcaagggttat

SEQ ID NO: 88 - DNA, 235 bp, Musca domestica
  source 1..235: mol_type = genomic DNA
cacctgcgtcatctagatgcggtgggctctatggccagagaacaacaacaagtggcttattttgatacttatgcgccacttgctacttatgagtacaattgtgctttgccacttgaacaaaaaattcattgattcatcgacactcgggtatgttttgtcgtgtcgttctgcgcactcagttaaattttttgtcttactctcttgctctcagcacatcaagtgttgttacttgttg

SEQ ID NO: 89 - DNA, 203 bp, Musca domestica
  source 1..203: mol_type = genomic DNA
tcgcacacgtccacttgtgagtttgtacactttttactctctcatactctagcggtgatcttaacatcaaacaactgttgttgttaagttgtgaaaaaatactcgtgtataaaaaaatacttgcactcaaaaggcttgacacccaaaacacttgtgcttatctatgtggcttacgtttgcctgtggcttgttgaagttctctg

SEQ ID NO: 90 - DNA, 24 bp, Tribolium castaneum
  source 1..24: mol_type = genomic DNA
gtataaagcagtgttcttcaacct

SEQ ID NO: 91 - DNA, 21 bp, Tribolium castaneum
  source 1..21: mol_type = genomic DNA
taggccagtgttcttcaacct

SEQ ID NO: 92 - DNA, 226 bp, Salmo salar
  source 1..226: mol_type = genomic DNA
agttgaagtcggaagtttacatacacttaagttggagtcattaaaactcgtttttcaactacaccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaa

SEQ ID NO: 93 - DNA, 228 bp, Salmo salar
  source 1..228: mol_type = genomic DNA
ttgagtgtatgttaacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaatgtatttggctaaggtgtatgtaaacttccgacttcaactg

SEQ ID NO: 94 - DNA, 200 bp, Oryzias latipes
  source 1..200: mol_type = genomic DNA
cagaggtgtaaagtacttgagtaattttacttgattactgtacttaagtattatttttggggatttttactttacttgagtacaattaaaaatcaatacttttacttttacttaattacatttttttagaaaaaaaagtactttttactccttacaattttatttacagtcaaaaagtacttattttttggagatcactt

SEQ ID NO: 95 - DNA, 150 bp, Oryzias latipes
  source 1..150: mol_type = genomic DNA
aatactcaagtacaattttaatggagtacttttttacttttactcaagtaagattctagccagatacttttacttttaattgagtaaaattttccctaagtacttgtactttcacttgagtaaaatttttgagtactttttacacctctg

SEQ ID NO: 96 - protein, 1171 aa, synthetic construct
MASTSRLDALPRVTCPNHPDAILVEDYRAGDMICPECGLVVGDRVIDVGSEWRTFSNDKATKDPSRVGDSQNPLLSDGDLSTMIGKGTGAASFDEFGNSKYQNRRTMSSSDRAMMNAFKEITTMADRINLPRNIVDRTNNLFKQVYEQKSLKGRANDAIASACLYIACRQEGVPRTFKEICAVSRISKKEIGRCFKLILKALETSVDLITTGDFMSRFCSNLCLPKQVQMAATHIARKAVELDLVPGRSPISVAAAAIYMASQASAEKRTQKEIGDIAGVADVTIRQSYRLIYPRAPDLFPTDFKFDTPVDKLPQLGGGGSGGGGSGGGGSGGGGSGGGGSNKGRKRTLRSRSRPLAHKRGTRAVEPEDPRPDSQGQRGGPKPGRYEPYDWDSDSDGGHRDGGGHGRVGRKTRAGRRDKAREPDAAAVEPVGPRHDSEGQRGAPKPGRYEPYDWDSDSDGGDGGGHGPPRDEALEPDAAAVEPEGPRHDSEGQGGGDGDRGGHGQVGRETLEDRRSDPPRDEALEPDAAAVEPEGPRHDSEGQGGGDGDRGGHGQVGRETLEDRRSDPPRDEAHEPDDGAVEPGGPQHELEGQGRGDEPSDWDLYECETDVDELDGSQSETEEQHRGPRSSGEEDVEEAVEEDAEEDIEEEPAAESASVYPDTFSSKNGEIKWSANERDRSSVRASPPHQASGPTTEAREGAQDILSSFLLFFTPNVERLILIATNRHSADKAHALKTPKPMDEIELRAYVGLLILAGVYRSRGEALVSLWEPECGRPIFGATMSLRSFYQRSATLRFDDRSTRASRSASDRLAAVREVWEEWCDRLPRLYDIGPEVTVDERMVPFKGRCSFRQYIPSKPTKYGLKLWVACDARTSYAWRVQPYMGKLASGARETNLASRVVSDLTRGLEHRNVTCDNFFTSYALATELLKRNITLLGTIKSNKPELPPELASARRREALSSRFAFTDCAAALSYVPKKNKNVLLLSTKHSRVEICHERRDKKPEMILDYNKTKGGVDNLDKLLATYSCRRMTKRWPLAMFHNMIDMSAYNAYVIWREMHPDWMPWNRNRRRIFLEQLGKDLVRPLVLRRKRVPRARPAYDLMVSMQEAGGTSARSEGKRKRCQICPRSKDTKTLISCKSCGKSICRGCTVPFCPLCSERQGRDGDTDASQ

SEQ ID NO: 97 - protein, 1014 aa, synthetic construct
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKFGGGGSGGGGSGGGGSGGGGSGGGGSANKGRKRTLRSRSRPLAHKRGTRAVEPEDPRPDSQGQRGGPKPGRYEPYDWDSDSDGGHRDGGGHGRVGRKTRAGRRDKAREPDAAAVEPVGPRHDSEGQRGAPKPGRYEPYDWDSDSDGGDGGGHGPPRDEALEPDAAAVEPEGPRHDSEGQGGGDGDRGGHGQVGRETLEDRRSDPPRDEALEPDAAAVEPEGPRHDSEGQGGGDGDRGGHGQVGRETLEDRRSDPPRDEAHEPDDGAVEPGGPQHELEGQGRGDEPSDWDLYECETDVDELDGSQSETEEQHRGPRSSGEEDVEEAVEEDAEEDIEEEPAAESASVYPDTFSSKNGEIKWSANERDRSSVRASPPHQASGPTTEAREGAQDILSSFLLFFTPNVERLILIATNRHSADKAHALKTPKPMDEIELRAYVGLLILAGVYRSRGEALVSLWEPECGRPIFGATMSLRSFYQRSATLRFDDRSTRASRSASDRLAAVREVWEEWCDRLPRLYDIGPEVTVDERMVPFKGRCSFRQYIPSKPTKYGLKLWVACDARTSYAWRVQPYMGKLASGARETNLASRVVSDLTRGLEHRNVTCDNFFTSYALATELLKRNITLLGTIKSNKPELPPELASARRREALSSRFAFTDCAAALSYVPKKNKNVLLLSTKHSRVEICHERRDKKPEMILDYNKTKGGVDNLDKLLATYSCRRMTKRWPLAMFHNMIDMSAYNAYVIWREMHPDWMPWNRNRRRIFLEQLGKDLVRPLVLRRKRVPRARPAYDLMVSMQEAGGTSARSEGKRKRCQICPRSKDTKTLISCKSCGKSICRGCTVPFCPLCSERQGRDGDTDASQ

SEQ ID NO: 98 - protein, 967 aa, synthetic construct
MSVNLNSERQFEDAASGDVQTYQIRTAPTSTIAPGVVMASSPALPTQPAEEAARKREVRLMKNREAARECRRKKKEYVKCLENRVAVLENQNKTLIEELKALKDLYCHKSDKGGGGSGGGGSGGGGSGGGGSGGGGSNKGRKRTLRSRSRPLAHKRGTRAVEPEDPRPDSQGQRGGPKPGRYEPYDWDSDSDGGHRDGGGHGRVGRKTRAGRRDKAREPDAAAVEPVGPRHDSEGQRGAPKPGRYEPYDWDSDSDGGDGGGHGPPRDEALEPDAAAVEPEGPRHDSEGQGGGDGDRGGHGQVGRETLEDRRSDPPRDEALEPDAAAVEPEGPRHDSEGQGGGDGDRGGHGQVGRETLEDRRSDPPRDEAHEPDDGAVEPGGPQHELEGQGRGDEPSDWDLYECETDVDELDGSQSETEEQHRGPRSSGEEDVEEAVEEDAEEDIEEEPAAESASVYPDTFSSKNGEIKWSANERDRSSVRASPPHQASGPTTEAREGAQDILSSFLLFFTPNVERLILIATNRHSADKAHALKTPKPMDEIELRAYVGLLILAGVYRSRGEALVSLWEPECGRPIFGATMSLRSFYQRSATLRFDDRSTRASRSASDRLAAVREVWEEWCDRLPRLYDIGPEVTVDERMVPFKGRCSFRQYIPSKPTKYGLKLWVACDARTSYAWRVQPYMGKLASGARETNLASRVVSDLTRGLEHRNVTCDNFFTSYALATELLKRNITLLGTIKSNKPELPPELASARRREALSSRFAFTDCAAALSYVPKKNKNVLLLSTKHSRVEICHERRDKKPEMILDYNKTKGGVDNLDKLLATYSCRRMTKRWPLAMFHNMIDMSAYNAYVIWREMHPDWMPWNRNRRRIFLEQLGKDLVRPLVLRRKRVPRARPAYDLMVSMQEAGGTSARSEGKRKRCQICPRSKDTKTLISCKSCGKSICRGCTVPFCPLCSERQGRDGDTDASQ

SEQ ID NO: 99 - protein, 2691 aa, synthetic construct
MMANSTGKAPPDERRKGLAFLDELRQFHHSRGSPFKKIPAVGGKELDLHGLYTRVTTLGGFAKVSEKNQWGEIVEEFNFPRSCSNAAFALKQYYLRYLEKYEKVHHFGEDDDEVPPGNPKPQLPIGAIPSSYNYQQHSVSDYLRQSYGLSMDFNSPNDYNKLVLSLLSGLPNEVDFAINVCTLLSNESKHVMQLEKDPKIITLLLANAGVFDDTLGSFSTVFGEEWKEKTDRDFVKFWKDIVDDNEVRDLISDRNKSHEGTSGEWIWESLFHPPRKLGINDIEGQRVLQIAVILRNLSFEEGNVKLLAANRTCLRFLLLSAHSHFISLRQLGLDTLGNIAAELLLDPVDFKTTHLMFHTVTKCLMSRDRFLKMRGMEILGNLCKAEDNGVLICEYVDQDSYREIICHLTLPDVLLVISTLEVLYMLTEMGDVACTKIAKVEKSIDMLVCLVSMDIQMFGPDALAAVKLIEHPSSSHQMLSEIRPQAIEQVQTQTHVASAPASRAVVAQHVAPPPGIVEIDSEKFACQWLNAHFEVNPDCSVSRAEMYSEYLSTCSKLARGGILTSTGFYKCLRTVFPNHTVKRVEDSSSNGQAHIHVVGVKRRAIPLPIQMYYQQQPVSTSVVRVDSVPDVSPAPSPAGIPHGSQTIGNHFQRTPVANQSSNLTATQMSFPVQGVHTVAQTVSRIPQNPSPHTHQQQNAPVTVIQSKAPIPCEVVKATVIQNSIPQTGVPVSIAVGGGPPQSSVVQNHSTGPQPVTVVNSQTLLHHPSVIPQQSPLHTVVPGQIPSGTPVTVIQQAVPQSHMFGRVQNIPACTSTVSQGQQLITTSPQPVQTSSQQTSAGSQSQDTVIIAPPQYVTTSASNIVSATSVQNFQVATGQMVTIAGVPSPQASRVGFQNIAPKPLPSQQVSSTVVQQPIQQPQQPTQQSVVIVSQPAQQGQTYAPAIHQIVLANPAALPAGQTVQLTGQPNITPSSSPSPVPATNNQVPTAMSSSSTPQSQGPPPTVSQMLSVKRQQQQQHSPAPPPQQVQVQVQQPQQVQMQVQPQQSNAGVGQPASGESSLIKQLLLPKRGPSTPGGKLILPAPQIPPPNNARAPSPQVVYQVASNQAAGFGVQGQTPAQQLLVGQQNVQLVPSAMPPSGGVQTVPISNLQILPGPLISNSPATIFQGTSGNQVTITVVPNTSFAPATVSQGNATQLIAPAGITMSGTQTGVGLPVQTLPATQASPAGQSSCTTATPPFKGDKIICQKEEEAKEATGLHVHERKIEVMENPSCRRGATNTSNGDTKENEMHVGSLLNGRKYSDSSLPPSNSGKIQSETNQCSLISNGPSLELGENGASGKQNSEQIDMQDIKSDLRKPLVNGICDFDKGDGSHLSKNIPNHKTSNHVGNGEISPMEPQGTLDITQQDTAKGDQLERISNGPVLTLGGSSVSSIQEASNAATQQFSGTDLLNGPLASSLNSDVPQQRPSVVVSPHSTTSVIQGHQIIAVPDSGSKVSHSPALSSDVRSTNGTAECKTVKRPAEDTDRETVAGIPNKVGVRIVTISDPNNAGCSATMVAVPAGADPSTVAKVAIESAVQQKQQHPPTYVQNVVPQNTPMPPSPAVQVQGQPNSSQPSPFSGSSQPGDPMRKPGQNFMCLWQSCKKWFQTPSQVFYHAATEHGGKDVYPGQCLWEGCEPFQRQRFSFITHLQDKHCSKDALLAGLKQDEPGQAGSQKSSTKQPTVGGTSSTPRAQKAIVNHPSAALMALRRGSRNLVFRDFTDEKEGPITKHIRLTAALILKNIGKYSECGRRLLKRHENNLSVLAISNMEASSTLAKCLYELNFTVQSKEQEKDSEMLQGGGGSGGGGSGGGGSGGGGSGGGGSNKGRKRTLRSRSRPLAHKRGTRAVEPEDPRPDSQGQRGGPKPGRYEPYDWDSDSDGGHRDGGGHGRVGRKTRAGRRDKAREPDAAAVEPVGPRHDSEGQRGAPKPGRYEPYDWDSDSDGGDGGGHGPPRDEALEPDAAAVEPEGPRHDSEGQGGGDGDRGGHGQVGRETLEDRRSDPPRDEALEPDAAAVEPEGPRHDSEGQGGGDGDRGGHGQVGRETLEDRRSDPPRDEAHEPDDGAVEPGGPQHELEGQGRGDEPSDWDLYECETDVDELDGSQSETEEQHRGPRSSGEEDVEEAVEEDAEEDIEEEPAAESASVYPDTFSSKNGEIKWSANERDRSSVRASPPHQASGPTTEAREGAQDILSSFLLFFTPNVERLILIATNRHSADKAHALKTPKPMDEIELRAYVGLLILAGVYRSRGEALVSLWEPECGRPIFGATMSLRSFYQRSATLRFDDRSTRASRSASDRLAAVREVWEEWCDRLPRLYDIGPEVTVDERMVPFKGRCSFRQYIPSKPTKYGLKLWVACDARTSYAWRVQPYMGKLASGARETNLASRVVSDLTRGLEHRNVTCDNFFTSYALATELLKRNITLLGTIKSNKPELPPELASARRREALSSRFAFTDCAAALSYVPKKNKNVLLLSTKHSRVEICHERRDKKPEMILDYNKTKGGVDNLDKLLATYSCRRMTKRWPLAMFHNMIDMSAYNAYVIWREMHPDWMPWNRNRRRIFLEQLGKDLVRPLVLRRKRVPRARPAYDLMVSMQEAGGTSARSEGKRKRCQICPRSKDTKTLISCKSCGKSICRGCTVPFCPLCSERQGRDGDTDASQ

SEQ ID NO: 100 - protein, 720 aa, synthetic construct
MSVNLNSERQFEDAASGDVQTYQIRTAPTSTIAPGVVMASSPALPTQPAEEAARKREVRLMKNREAARECRRKKKEYVKCLENRVAVLENQNKTLIEELKALKDLYCHKSDKGGGGSGGGGSGGGGSGGGGSGGGGSGSSYPFALASEPDEEIQETLDEDDDSEEDDVEDVSNDLEEELTDSDDDGSESEKSDFVIEQPGSSLASNRILTLPQRTIRGKNKHCWSTSKSTRRSRVSALNIVRSQRGPTRMCRNIYDPLLCFKLFFTDEIISEIVKHTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRQTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFLKELYMSLTSSFMRKRLEAPTLKRYLRDNISNILPNEVPGTSDDSTEEPVMKKRTYCTYCPSKIRRKANASCKKCKKVICREHNIDMCQSCF